(19) United States
(12) Patent Application Publication
(10) Pub. No.: US 2026/0327561 A1
(43) Pub. Date: Oct. 22, 2026

(54) METHODS AND SYSTEMS FOR PREDICTING GENE EDITING TIMING

(71) Applicant: WISCONSIN ALUMNI RESEARCH FOUNDATION, Madison, WI (US)

(72) Inventors: Melissa C. SKALA, Madison, WI (US); Dan L. PHAM, Madison, WI (US); Dan CAPPABIANCA, Madison, WI (US); Krishanu SAHA, Madison, WI (US)

(21) Appl. No.: 19/480,578

(22) PCT Filed: May 1, 2024
(86) PCT No.: PCT/US2024/027216

§ 371 Date: Oct. 31, 2025

Related U.S. Application Data
(60) Provisional application No. 63/499,334, filed on May 1, 2023.

(51) Int. Cl. C12N 5/0783; C12M 1/34; C12M 1/42; C12N 9/22; C12N 15/11; C12N 15/90; G01N 21/64

(52) U.S. Cl. CPC C12N 5/0636; C12M 35/02; C12M 41/38; C12N 9/226; C12N 15/11; C12N 15/907; G01N 21/6428; C12N 2310/20; C12N 2501/515; C12N 2501/53; C12N 2501/599; C12N 2510/00; C12N 2529/10

(57) ABSTRACT

Methods and systems for gene editing of a T cell. The method includes: stimulating a T cell obtained from a subject; obtaining a plurality of measurements of a metabolic indicator within the T cell at a respective plurality of timepoints after stimulating the T cell; analyzing the plurality of measurements of the metabolic indicator to identify a particular time for performing gene editing of the T cell; and providing an indication to perform gene editing of the T cell at the particular time based on analyzing the measurements.

### CROSS-REFERENCE TO RELATED APPLICATIONS

This application is related to, claims priority to, and incorporates herein by reference for all purposes U.S. Provisional Patent Application No. 63/499,334, filed May 1, 2023.

### STATEMENT REGARDING FEDERALLY FUNDED RESEARCH

This invention was made with government support under EEC1648035 awarded by the National Science Foundation and under GM119644 awarded by the National Institutes of Health. The government has certain rights in the invention.

### BACKGROUND

While Chimeric Antigen Receptor (CAR) T therapy is a promising cancer treatment that has achieved decade-long remission for hematological malignancies, its translation to solid tumor treatment remains challenging. The CAR T therapy procedure starts with removal of the patient's own T cells from their blood and sending the cells to a lab where the cells' genes are altered such that the cells produce proteins called chimeric antigen receptors (CARs) on their surface. These special receptors allow the T cells to identify and attack cancer cells. The altered CAR+ T cells are multiplied and grown in a lab after which the CAR T cells are infused into the patient's blood.

The number of CAR+ cells (CAR T dosage) and stem-memory cell phenotypes have been correlated with long term treatment response. Low CAR knock-in efficiency can be compensated by longer in vitro expansion to reach the desired dosage, but extended culture can cause differentiation and exhaustion of CAR T cells. Therefore, strategies to improve CAR knock-in efficiency and expansion conditions to establish a potent, persistent CAR T therapy are important for advancing this therapeutic approach.

### SUMMARY

Accordingly, one embodiment provides a method for gene editing of a T cell, including: stimulating a T cell obtained from a subject; obtaining a plurality of measurements of a metabolic indicator within the T cell at a respective plurality of timepoints after stimulating the T cell; analyzing the plurality of measurements of the metabolic indicator to identify a particular time for performing gene editing of the T cell; and providing an indication to perform gene editing of the T cell at the particular time based on analyzing the measurements.

In some embodiments, stimulating a T cell obtained from a subject further includes stimulating a T cell obtained from a subject by contacting the T cell with at least one of ImmunoCult (αCD2/αCD3/αCD28) or TransAct (αCD3/αCD28) antibody reagents. In other embodiments obtaining a plurality of measurements further includes obtaining a plurality of autofluorescence measurements from the T cell. In still further embodiments, obtaining a plurality of autofluorescence measurements further includes stimulating the T cell using excitation light, and obtaining at least one of photon counts/intensity or fluorescence lifetimes from the T cell. In particular embodiments, stimulating the T cell using excitation light further includes stimulating the T cell using excitation light configured to be absorbed by at least one of NAD(P)H or FAD, and obtaining a plurality of autofluorescence measurements further includes obtaining the plurality of autofluorescence measurements from at least one of NAD(P)H or FAD stimulated by excitation light in the T cell.

In certain embodiments, obtaining a plurality of measurements of a metabolic indicator within the T cell at a respective plurality of timepoints after stimulating the T cell further includes obtaining a plurality of measurements of autofluorescence decay from the T cell based on the plurality of measurements obtained at the respective plurality of timepoints, where the autofluorescence decay comprises signal from at least one of NAD(P)H or FAD within the T cell, and analyzing the plurality of measurements of the metabolic indicator to identify a particular time for performing gene editing of the T cell further includes identifying the metabolic indicator based on analyzing the autofluorescence decay from the T cell.

In some embodiments, identifying the metabolic indicator based on analyzing the autofluorescence decay from the T cell further includes identifying the metabolic indicator based on using at least one of a curve-fitting or fit-free analysis of the autofluorescence decay, where the autofluorescence decay is modeled as a multi-component exponential decay.

In particular embodiments, the metabolic indicator includes at least one of NAD(P)H mean fluorescence lifetime (τₘ) or NAD(P)H shortest fluorescence amplitude component (α₁), and analyzing the plurality of measurements of the metabolic indicator to identify a particular time for performing gene editing of the T cell further includes obtaining a plurality of values of NAD(P)H τₘ and a plurality of values of NAD(P)H α₁ at each of the respective plurality of timepoints, analyzing the plurality of values of NAD(P)H τₘ and the plurality of values of NAD(P)H α₁, and identifying the particular time for performing gene editing of the T cell based on analyzing the plurality of values of NAD(P)H τₘ and the plurality of values of NAD(P)H α₁.

In further embodiments, analyzing the plurality of values of NAD(P)H τₘ and the plurality of values of NAD(P)H α₁ further includes identifying at least one of a minimum value of the plurality of values of NAD(P)H τₘ or a maximum value of the plurality of values of NAD(P)H α₁, and identifying the particular time for performing gene editing of the T cell based on identifying at least one of a minimum value of the plurality of values of NAD(P)H τₘ or a maximum value of the plurality of values of NAD(P)H α₁.

In particular embodiments, the particular time for performing gene editing of the T cell corresponds to a time when the T cell is at the G2/M cell cycle phase. In certain embodiments, performing gene editing of the T cell at the particular time based on analyzing the measurements further includes adding a chimeric antigen receptor to the T cell using at least one of CRISPR or viral editing gene editing procedures.

In some embodiments, obtaining a plurality of autofluorescence measurements from the T cell further includes obtaining a plurality of measurements of a cytoplasm size or a cell size for the T cell at each of the plurality of timepoints, identifying a maximum value of the cytoplasm size or the cell size on the plurality of measurements of the cytoplasm size or the cell size, and identifying the particular time for performing gene editing based on identifying the maximum value of the cytoplasm size or the cell size.

In some embodiments, the method for gene editing of a T cell further includes infusing the T cell into the subject.

In other embodiments, obtaining a plurality of measurements of a metabolic indicator within the T cell further includes obtaining the plurality of measurements of the metabolic indicator within the T cell while the T cell is contained within an observation zone. In some embodiments, the observation zone is part of at least one of a flow sorter, a flow cytometer, an imaging cytometer, or a microscope. In other embodiments, the observation zone is part of an aseptic and non-destructive cell handling system.

Another embodiment provides a system for gene editing of a T cell, including: an observation zone comprising a T cell obtained from a subject, the T cell being stimulated at an initial time; a spectrometer configured to obtain a plurality of measurements of a metabolic indicator within the T cell at a respective plurality of timepoints after stimulating the T cell at the initial time; a processor in electronic communication with the spectrometer; and a non-transitory computer-readable medium accessible to the processor and having stored thereon instructions that, when executed by the processor, cause the processor to: analyze the plurality of measurements of the metabolic indicator to identify a particular time for performing gene editing of the T cell, and provide an indication of the particular time to perform gene editing of the T cell based on analyzing the measurements.

In some embodiments, the T cell obtained from the subject is stimulated by contacting the T cell with at least one of ImmunoCult (αCD2/αCD3/αCD28) or TransAct (αCD3/αCD28) antibody reagents. In other embodiments, the spectrometer, when obtaining a plurality of measurements, is further configured to obtain a plurality of autofluorescence measurements from the T cell. In still further embodiments, the spectrometer, when obtaining a plurality of autofluorescence measurements, is further configured to stimulate the T cell using excitation light, and obtain at least one of photon counts/intensity or fluorescence lifetimes from the T cell.

In particular embodiments, the spectrometer, when stimulating the T cell using excitation light, is further configured to stimulate the T cell using excitation light configured to be absorbed by at least one of NAD(P)H or FAD, and wherein the spectrometer, when obtaining a plurality of autofluorescence measurements, is further configured to obtain the plurality of autofluorescence measurements from at least one of NAD(P)H or FAD stimulated by excitation light in the T cell.

In certain embodiments, the spectrometer, when obtaining a plurality of measurements of a metabolic indicator within the T cell at a respective plurality of timepoints after stimulating the T cell, is further configured to obtain a plurality of measurements of autofluorescence decay from the T cell based on the plurality of measurements obtained at the respective plurality of timepoints, wherein the autofluorescence decay includes signal from at least one of NAD(P)H or FAD within the T cell, and wherein the processor, when analyzing the plurality of measurements of the metabolic indicator to identify a particular time for performing gene editing of the T cell, is further caused by the instructions to identify the metabolic indicator based on analyzing the autofluorescence decay from the T cell.

In some embodiments, the processor, when identifying the metabolic indicator based on analyzing the autofluorescence decay from the T cell, is further caused by the instructions to identify the metabolic indicator based on using at least one of a curve-fitting or fit-free analysis of the autofluorescence decay, wherein the autofluorescence decay is modeled as a multi-component exponential decay.

In certain embodiments, the metabolic indicator includes at least one of NAD(P)H mean fluorescence lifetime (τₘ) or NAD(P)H shortest fluorescence amplitude component (α₁), and wherein the processor, when analyzing the plurality of measurements of the metabolic indicator to identify a particular time for performing gene editing of the T cell, is further caused by the instructions to obtain a plurality of values of NAD(P)H τₘ and a plurality of values of NAD(P)H α₁ at each of the respective plurality of timepoints, analyze the plurality of values of NAD(P)H τₘ and the plurality of values of NAD(P)H α₁, and identify the particular time for performing gene editing of the T cell based on analyzing the plurality of values of NAD(P)H τₘ and the plurality of values of NAD(P)H α₁.

In further embodiments, the processor, when analyzing the plurality of values of NAD(P)H τₘ and the plurality of values of NAD(P)H α₁, is further caused by the instructions to identify at least one of a minimum value of the plurality of values of NAD(P)H τₘ or a maximum value of the plurality of values of NAD(P)H α₁, and identify the particular time for performing gene editing of the T cell based on identifying at least one of a minimum value of the plurality of values of NAD(P)H τₘ or a maximum value of the plurality of values of NAD(P)H α₁.

In some embodiments, the particular time for performing gene editing of the T cell corresponds to a time when the T cell is at the G2/M cell cycle phase. In further embodiments, the processor, when providing an indication of the particular time to perform gene editing of the T cell, is further caused by the instructions to provide an instruction to add a chimeric antigen receptor to the T cell using at least one of CRISPR or viral editing gene editing procedures. In still further embodiments, the spectrometer, when obtaining a plurality of autofluorescence measurements from the T cell, is further configured to obtain a plurality of measurement of a cytoplasm or a cell size for the T cell at each of the plurality of timepoints, and where the processor, when analyzing the plurality of measurements of the metabolic indicator, is further caused by the instructions to identify a maximum value of the cytoplasm size or the cell size on the plurality of measurements of the cytoplasm size or the cell size, and identify the particular time for performing gene editing based on identifying the maximum value of the cytoplasm size or the cell size. In some embodiments, the T cell is infused into the subject.

In further embodiments, the spectrometer, when obtaining a plurality of measurements of a metabolic indicator within the T cell, is further configured to obtain the plurality of measurements of the metabolic indicator within the T cell while the T cell is contained within the observation zone. In particular embodiments, the observation zone is part of at least one of a flow sorter, a flow cytometer, an imaging cytometer, or a microscope. In certain embodiments, the observation zone includes an aseptic and non-destructive cell handling system.

Yet another embodiment provides a method for gene editing of a T cell, including: stimulating a T cell obtained from a subject; obtaining a plurality of measurements indicative of a cell cycle state of the T cell at a respective plurality of timepoints after stimulating the T cell; analyzing the plurality of measurements indicative of the cell cycle state to identify a particular time for performing gene editing of the T cell; and providing an indication to perform gene editing of the T cell at the particular time based on analyzing the measurements.

In some embodiments, stimulating a T cell obtained from a subject further includes stimulating a T cell obtained from a subject by contacting the T cell with at least one of ImmunoCult (αCD2/αCD3/αCD28) or TransAct (αCD3/αCD28) antibody reagents. In other embodiments, obtaining a plurality of measurements further includes obtaining a plurality of optical measurements from the T cell. In other embodiments, obtaining a plurality of optical measurements from the T cell further includes obtaining a plurality of measurements of a cytoplasm size or a cell size for the T cell at each of the plurality of timepoints based on the plurality of optical measurements, identifying a maximum value of the cytoplasm size or the cell size based on the plurality of measurements of the cytoplasm size or the cell size, and identifying the particular time for performing gene editing based on identifying the maximum value of the cytoplasm size or the cell size.

In some embodiments, the particular time for performing gene editing of the T cell corresponds to a time when the T cell is at the G2/M cell cycle phase. In other embodiments, obtaining a plurality of optical measurements further includes obtaining a plurality of autofluorescence measurements from the T cell. In still further embodiments, obtaining a plurality of autofluorescence measurements further includes stimulating the T cell using excitation light, and obtaining at least one of photon counts/intensity or fluorescence lifetimes from the T cell.

In other embodiments, stimulating the T cell using excitation light further includes stimulating the T cell using excitation light configured to be absorbed by at least one of NAD(P)H or FAD, and wherein obtaining a plurality of measurements indicative of a cell cycle state of the T cell further includes obtaining the plurality of autofluorescence measurements from at least one of NAD(P)H or FAD stimulated by excitation light in the T cell.

In other embodiments, performing gene editing of the T cell at the particular time based on analyzing the measurements further includes adding a chimeric antigen receptor to the T cell using at least one of CRISPR or viral editing gene editing procedures. In some embodiments, the method for gene editing of a T cell includes infusing the T cell into the subject. In some embodiments, obtaining a plurality of measurements indicative of a cell cycle state of the T cell further includes obtaining a plurality of measurements indicative of a cell cycle state of the T cell while the T cell is contained within an observation zone. In other embodiments, the observation zone is part of at least one of a flow sorter, a flow cytometer, an imaging cytometer, or a microscope. In other embodiments, the observation zone is part of an aseptic and non-destructive cell handling system.

Still another embodiment provides a system for gene editing of a T cell, including: an observation zone comprising a T cell obtained from a subject, the T cell being stimulated at an initial time; a spectrometer configured to obtain a plurality of measurements indicative of a cell cycle state of the T cell at a respective plurality of timepoints after stimulating the T cell at the initial time; a processor in electronic communication with the spectrometer; and a non-transitory computer-readable medium accessible to the processor and having stored thereon instructions that, when executed by the processor, cause the processor to: stimulate a T cell obtained from a subject; obtain a plurality of measurements indicative of a cell cycle state of the T cell at a respective plurality of timepoints after stimulating the T cell; analyze the plurality of measurements indicative of the cell cycle state to identify a particular time for performing gene editing of the T cell; and provide an indication to perform gene editing of the T cell at the particular time based on analyzing the measurements.

In some embodiments, the T cell obtained from the subject is stimulated by contacting the T cell with at least one of ImmunoCult (αCD2/αCD3/αCD28) or TransAct (αCD3/αCD28) antibody reagents. In further embodiments, the spectrometer, when obtaining a plurality of measurements, is further configured to obtain a plurality of optical measurements from the T cell.

In certain embodiments, the spectrometer, when obtaining a plurality of optical measurements from the T cell, is further configured to obtain a plurality of measurements of a cytoplasm size or the cell size for the T cell at each of the plurality of timepoints based on the plurality of optical measurements, identify a maximum value of the cytoplasm size or the cell size based on the plurality of measurements of the cytoplasm size or the cell size, and identify the particular time for performing gene editing based on identifying the maximum value of the cytoplasm size or the cell size.

In some embodiments, the particular time for performing gene editing of the T cell corresponds to a time when the T cell is at the G2/M cell cycle phase. In other embodiments, the spectrometer, when obtaining a plurality of optical measurements, is further configured to obtain a plurality of autofluorescence measurements from the T cell. In still further embodiments, the spectrometer, when obtaining a plurality of autofluorescence measurements, is further configured to stimulate the T cell using excitation light, and obtain at least one of photon counts/intensity or fluorescence lifetimes from the T cell.

In some embodiments, the spectrometer, when stimulating the T cell using excitation light, is further configured to stimulate the T cell using excitation light configured to be absorbed by at least one of NAD(P)H or FAD, and wherein the spectrometer, when obtaining a plurality of measurements indicative of a cell cycle state of the T cell, is further configured to: obtain the plurality of autofluorescence measurements from at least one of NAD(P)H or FAD stimulated by excitation light in the T cell. In other embodiments, the processor, when providing an indication of the particular time to perform gene editing of the T cell, is further caused by the instructions to provide an instruction to add a chimeric antigen receptor to the T cell using at least one of CRISPR or viral editing gene editing procedures. In particular embodiments, the spectrometer, when obtaining a plurality of measurements indicative of a cell cycle state of the T cell, is further configured to obtain a plurality of measurements indicative of a cell cycle state of the T cell while the T cell is contained within an observation zone.

### BRIEF DESCRIPTIONS OF THE DRAWINGS

FIG. 1A shows an example of a process for gene editing of a T cell in accordance with some embodiments of the disclosed subject matter.

FIG. 1B shows a block diagram of a system, in accordance with an aspect of the present disclosure.

FIG. 2 shows a diagram of one specific implementation of a system, in accordance with aspects of the present disclosure.

FIG. 3 shows significant changes in CD3 T cell metabolism and morphology post activation that were characterized by OMI. (Panels A, B, and C) Compared to quiescent cells, T cells activated with Imm method (top row) or Tex method (bottom row) showed significantly (Panel A) lower NAD(P)H τₘ, (Panel B) higher free NAD(P)H fraction (NAD(P)H α₁), and (Panel C) larger cytoplasm size, n=5929 cells from 3 independent donors. Solid lines represent donor average. (Panels D, E, F, and G) T cells subjected to different activation methods showed distinct OMI metabolic profile. (Panels D, E, and F) Uniform Manifold Approximation Projection (UMAP) based on Euclidean distance of 11 OMI parameters (NAD(P)H τₘ, τ₁, τ₂, α₁, α₂, FAD τₘ, τ₁, τ₂, α₁, α₂, cytoplasm size) showed distinct clustering of T cells activated with Imm or Tex methods from 3 donors, n=5929 cells. (Panel G) Three classification models (Support Vector Machine, Random Forest and Logistic Regression) were trained to classify activated T cells by activation methods (Imm versus Tex) based on 14 OMI parameters (13 metabolic features (Table 2)+cytoplasm/cell size). Data were randomly split into 80% for training and 20% for testing (n=4743 cells for trainings, 1186 for testing). Receiver operating characteristic (ROC) curve and area under the curve (AUC) were calculated for each classification method, AUC=1 represents 100% classification accuracy for test data set. Scale bar is 50 μm. Error bar is SD.

FIG. 4 shows that OMI was sensitive to metabolic changes as T cells progressed through the cell cycle following activation and allowed identification of an optimal time frame for genome editing via CRISPR/Cas9. (Panels A-B) T cells activated using (Panel A) the Imm method and (Panel B) the Tex method showed significantly higher proportion of cells in G2/M/S phase at 72 hours compared to earlier timepoints. (Panels C-E) OMI parameters including (Panel C) NAD(P)H τₘ, (Panel D) NAD(P)H α₁ and (Panel E) cytoplasm size, showed significant and strong correlations with the percentage of cells in G2/M/S phase. Each dot represents one sample average, coded based on the method of activation (Imm versus Tex) and duration of activation (0-72 hours). n=40 samples including 7 activation durations, 2 activation methods and 3 donors. (Panels F-H) Activation durations and methods affected T cells' gene editing efficiency, as quantified by percentage of successful CAR incorporation. (Panel F) Imm activated T cells and (Panel G) Tex activated T cells showed peak editing efficiency with different activation duration, 48-60 hours and 60-72 hours respectively. (Panels H-J) OMI measurements, including (Panel H) NAD(P)H τₘ, (Panel I) NAD(P)H α₁, and (Panel J) cytoplasm size, of T cells at transfection timepoint showed significant and moderate-to-strong correlations to gene editing efficiency (% CAR). Quiescent T cells with 0 hour of activation duration did not undergo transfection. (Panel K) Cell cycle stage (% G2/M/S) at transfection timepoint showed strong and positive correlation to transfection outcome (% CAR). For (Panels H-K), n=35 samples including 6 activation durations, 2 activation methods and 3 donors. (Panel L) Three classification models were trained on 80% data and tested on 20% data to predict gene editing outcomes (high versus low efficiency) based on OMI metabolic and morphological profiles at transfection timepoint. 15% CAR for Imm activated T cells and 10% CAR for Tex activated T cells is the threshold for high editing efficiency (n=3881 cells for trainings, 970 cells for testing).

FIG. 5 shows additional data indicating that timing of gene editing correlates significantly with editing outcomes. (Panels A-C) Following activation with (Panel A) Imm method or (Panel B) Tex method, T cell proliferation increased (% Ki67), and (Panel C) proliferate rate at gene editing time point strongly correlated with gene editing outcome (% CAR). (Panels D-I) T cells with lower NAD(P)H τₘ, higher NAD(P)H α₁ and larger cytoplasm size at viral transduction time point showed significantly higher transduction efficiency. (Panel D) Experimental setup. T cells were isolated from peripheral blood of two healthy donor and activated with Imm method and underwent TCR (TRAC) knockout prior to transduction with retroviral to express GD-2 CAR receptor with two constructs (OX40-CD28-CAR or 41BB-CAR) following previously described methods (see Mueller K P, Piscopo N J, Forsberg M H, et al Production and characterization of virus-free, CRISPR-CAR T cells capable of inducing solid tumor regression Journal for ImmunoTherapy of Cancer 2022; 10:e004446. doi: 10.1136/jitc-2021-004446, which is incorporated herein by reference in its entirety). (Panel E) Representative NAD(P)H τₘ images of control and TRAC KO T cells at transduction. (Panels F-H) OMI measurements showed that TRAC KO T cells had significantly (Panel F) lower NAD(P)H τₘ, (Panel G) higher NAD(P)H α₁ and (Panel H) greater cytoplasm size at transduction. (Panel I) Compared to control cells, TRAC KO T cells yielded significantly higher transduction efficiency, quantified as the percentage of CAR+ T cells at harvest.

FIG. 6 shows significant correlations between normalized redox ratio and whole cell size compared to % G2/M/S and % CAR, n=40 samples for % G2/M/S correlation and n=35 samples for % CAR correlation. For each donor and activation method, redox ratios at different activation durations were normalized to donor-match T cells activated for 12 hours of the corresponding activation method (Tex or Imm).

FIG. 7. Activating media, not activating antibody, determines CD3 T cell metabolism. (A) Experimental timeline. (B) Representative NAD(P)H mean lifetime (NAD(P)H τₘ) images. (C, D) quantification of NAD(P)H τₘ of quiescent (left) and activated (right) CD3 T cells in either (C) ImmunoCult XF or (D) TexMACS media. n=93-290 cells/condition/donor, two-way ANOVA and adjusted multiple comparison. (E) ATP production and (F) reducing potential of T cells activated with TransAct αCD3/αCD28 antibody in ImmunoCult XF and TexMACS media. n=3 replicates/condition/timepoint, two-way ANOVA with Fisher's LSD post-hoc test for multiple comparison. (G, H) UMAP based on Euclidean distance projection of 14 OMI parameters (Table 2) from T cells activated for 24-72 hours, colors representing (G) culture media and (H) activating antibody. n=4,923 cells from 3 independent donors. (I) Receiver operating characteristic (ROC) curves and areas under the curve (AUCs) Random Forest (RF) and Logistic Regression (LR) algorithms to classify T cells activated in ImmunoCult XF versus TexMACS media (regardless of activating antibody or activation duration) based on OMI parameters. Data were randomly split into 70% for training (n=3,446 cells) and 30% for testing (n=1,477 cells). Bars are mean±SD. * p<0.0001.

FIG. 8. OMI is sensitive to metabolic changes as T cells progress through cell cycle following activation. (A) Experimental timeline. (B) Representative NAD(P)H τₘ images of T cells activated for 12-72 hours with Imm (top row) or Tex (bottom row) activation methods. Quantification of (C) NAD(P)H τₘ, (D) NAD(P)H α₁, and (E) cytoplasm size of T cells activated with Imm method (left column) or Tex method (right column). n=300-500 cells/condition from 3 donors, Brown-Forsythe and Welch ANOVA test with Dunnette T3 post hoc test. Glass's deltas were calculated with respect to quiescent cells as the effect sizes of Imm and Tex activation methods on OMI parameters. (F, G) Percentage of T cells in S/G2/M phase following activation with (F) Imm or (G) Tex method. n=3 donors, Brown-Forsythe and Welch ANOVA test with Dunnette T3 post hoc test. (H, I) Correlation between OMI parameters, including (H) NAD(P)H τₘ and (I) cytoplasm size, and the percentage of cells in S/G2/M phase. n=42 samples from 3 donors, Pearson R analysis. (B-G) Solid lines represent donor averages. (H, I) Each dot represents one sample average, coded based on the method (Imm or Tex) and duration of activation (0-72 hours). Bars are mean±SD. * p<0.0001.

FIG. 9. OMI identifies optimal timeframe for CRISPR/Cas9 genome editing. (A) Experimental timeline. (B, C) Genome editing efficiency (% CAR positivity) for T cells activated with (B) Imm and (C) Tex methods for 12-72 hours. n=4-5 replicates across 3 donors, Brown-Forsythe and Welch ANOVA test with Dunnette T3 post hoc test. (D-G) Pearson R correlation analysis among OMI parameters, Hoechst median fluorescence intensity (MFI) at electroporation, and genome editing efficiency post expansion. NAD(P)H intensity, FAD intensity, redox ratio and Hoechst MFI were normalized to 12-hour-activated group averages. n=36 samples across 3 donors. Crossed out cells represent non-significant correlations (p>0.05). (E-G) Each dot represents one sample average, coded by activation method (Imm or Tex) and duration of activation (12-72 hours). (H, I) ROCs and AUCs for classification models to predict gene editing outcomes, i.e. high efficiency (>15% CAR⁺ for Imm-activated T cells and >10% CAR⁺ for Tex-activated T cells) versus low efficiency, based on (H) OMI parameters and (I) normalized Hoechst MFIs at electroporation. n=3881 cells for training, 970 cells for testing for (H) and n=611,841 cells for training and 152,960 cells for testing for (I). Bars are mean±SD. * p<0.0001.

FIG. 10. Media composition has a significant impact on CAR T cell metabolism and phenotype post-expansion. (A) Experimental timeline. (B) Z-score heatmap based on 14 OMI parameters of CAR T cells (both CAR⁺ and CAR⁻) expanded in ImmunoCult XF (Imm) or TexMACS (Tex) media with various cytokines. Hierarchical clustering was determined based on Euclidean distance from population mean using Ward's criterion. (C) UMAP based on Euclidean distance of OMI metabolic parameters for CAR T cells at the end of manufacturing, color coded by (C) culture media or (D) supplemented cytokines. n=1,417 cells from 3 donors for (B-D). (D) ROC curves and AUCs of models to classify CAR T cells expanded in ImmunoCult XF or TexMACS media based on OMI parameters. n=1,134 cells for training and n=283 cells for testing. (E-F) UMAP based on Euclidean distance of surface marker median fluorescence intensity (MFI) (CD27, CD45RO, CD62L, CD28, CD45RA and CCR7). n=87,317 cells across 3 donors. (G) ROC curves and AUCs of RF and LR classifiers to classify T cells expanded in ImmunoCult XF or TexMACS media based on surface marker expression. n=52,390 cells for training; n=34,927 cells for testing.

FIG. 11. CAR T cells undergoing media transition showed a distinct OMI metabolic profile with lower glycolytic activity compared to those expanded in singular media. (A) Experimental timeline. (B) CCR7 (left) and CD62L (right) expression profile of pre-infusion CAR T cells. (C) Lactate production from 1 million CAR T cells within 24 hours. n=5-17 samples/condition, Brown-Forsythe and Welch ANOVA test with Dunnette T3 test for multiple comparisons against Tex→Imm group. (D) Representative NAD(P)H τₘ images and quantification of (E) NAD(P)H α₁ and (F) NAD(P)H τₘ of CAR T cells expanded in Imm, Tex, or Tex→Imm media conditions. Brown-Forsythe and Welch ANOVA test with Games-Howell's test for multiple comparisons. (G) NAD(P)H τₘ histogram of CAR T cells post expansion. R² value represents goodness of fit to a double Gaussian distribution. Percentages of cells in the high NAD(P)H τₘ Gaussian are noted. (E-G) n=143-345 cells/condition from 2 donors. (H) Representative immunofluorescence and NAD(P)H τₘ image and quantification of (I) NAD(P)H τₘ and (J) NAD(P)H α₁ from CCR7⁺ and CCR7⁻ CAR T cells expanded in the Tex→Imm media condition. n=371-519 cells/group across 4 donors, Mann-Whitney test. Bars are mean±SD. * p<0.0001.

FIG. 12. OMI metabolic features of CAR T cells pre-infusion predict in vivo potency. (A) Experimental timeline. (B) Representative images of NSG mice bearing GD2+ Luciferase CHLA-20 tumors before (top row) and 17 days after (bottom row) intravenous injection with 4 million CAR⁺ T cells/mouse. Red asterisks represent tumor-free mice on day 17. (C) Tumor flux in NSG mice before (day −1) and after (day 17) treatment with Imm, Tex→Imm, and Tex CAR T cells. n=2-5 mice/condition across 2 donors, paired non-parametric t-test (Wilcoxon test). (D, E) Expression of (D) stem central memory and (E) exhaustion markers in splenic T cells post treatment. 2D kernel density estimation plots based on MFIs of surface markers (D) CCR7 and CD62L and (e) PD-1 and TIGIT of CD45⁺ T cells isolated from treated mouse spleens after 21 days of CAR T treatment. n=9,569 cells from 11 mice across 2 donors. (F) UMAP projection based on Euclidean distance metric of OMI parameters for CAR T cells that were activated and expanded in Imm, Tex, or Tex→Imm conditions. n=648 cells from 2 donors. (G) ROC curves and corresponding AUCs of 2 models to classify high potency (Tex→Imm) versus low potency (Imm and Tex groups) CAR T cells based on OMI parameters of pre-infusion products. n=518 cells for training and n=130 cells for testing. * p<0.0001.

FIG. 13. Activation time-course and conditions for anti-GD2 CAR T cell generation with CRISPR/Cas9. CD3 T cells were isolated from 3 healthy donors and activated with StemCell αCD2/αCD3/αCD28 in ImmunoCult XF media (Imm) or TransAct αCD3/αCD28 in TexMACS media (Tex). Cells were divided into 7 groups with duration of activation ranging from 0 hours (quiescent T cells) to 72 hours prior to electroporation to introduce the anti-GD2 CAR transgene. When activated T cells were collected for electroporation, their metabolic features were characterized using OMI. Meanwhile, their cell cycle stage and proliferation capacity were assessed using flow cytometry of Hoechst 33342 and Ki-67 staining. These activated T cells were then electroporated to incorporate the anti-GD2 CAR transgene and expanded in corresponding culture media. Genome editing efficiency was determined after 7 days of expansion with GD2 CAR antibody using flow cytometry.

FIG. 14. T cells progressed through cell cycle and proliferated upon activation. (A) Experimental timeline. (B, C) T cells progressed through cell cycle upon activation. Density plots of Hoechst MFI in T cells activated with (B) Imm and (C) Tex methods. For each donor, Hoechst MFI was normalized to the average of 12-hour-activated group. n=393,999 cells and 370,802 cells for Imm and Tex activation methods, respectively. (D) Correlation between NAD(P)H α₁ and cell cycle stage (% cells in S/G2/M phase) at electroporation. Each dot represents one sample average, color coded based on the method of activation (Imm or Tex) and duration of activation (0-72 hours). n=42 samples, Pearson R analysis. (E, F) T cell proliferation (% Ki-67⁺) following activation with (E) Imm method or (F) Tex method. n=3 donors, Brown-Forsythe and Welch ANOVA test with Dunnett's post hoc test for multiple comparisons. Bars are mean±SD. Lines connect donor-match averages.

FIG. 15. T cell metabolism at viral transduction timepoint correlated with transduction efficiency. (A) Experimental setup. T cells were isolated from peripheral blood of two healthy donors and activated with Imm method. Activated CD3 T cells underwent electroporation with Cas9 ribonucleoproteins targeting the human TRAC locus to knockout the T cell receptor (TRAC KO) 2 days prior to transduction with retrovirus to express anti-GD2 CAR receptor using two constructs (OX40-CD28-CAR or 41BB-CAR) as previously described⁹. OMI was performed immediately before transduction. Transduction efficiency was quantified as % CAR⁺ after 7 days of expansion. (B) Representative NAD(P)H τₘ images of TCR-intact and TRAC KO T cells at transduction. OMI parameters including (C) NAD(P)H τₘ, (D) NAD(P)H α₁, and (E) cytoplasm size of cells with intact T cell receptor (TCR-intact) and TRAC KO T cells at transduction. n=374-480 cells/group from 2 independent donors, Mann-Whitney test. (F) Transduction efficiency (% CAR) of TCR-intact and TRAC KO cells at the end of the manufacturing process. n=17 samples from 2 donors and 2 anti-GD2 CAR constructs, unpaired T test. Bars are mean±SD. Lines connect donor-match averages. * p<0.0001.

FIG. 16. Cell characteristics at electroporation correlated with genome editing efficiency. (A) Experimental timeline. (B-E) Correlation between cell characteristics (B) NAD(P)H α₁, (C) normalized redox ratio, at electroporation and genome editing outcome (% CAR). Each dot represents one sample average, coded based on the method of activation (Imm or Tex) and duration of activation (0-72 hours). n=36 samples, Pearson R analysis. Quiescent T cells with 0-hour activation duration did not undergo genome editing.

FIG. 17. OMI revealed metabolic differences in CAR T cells expanded in Imm and Tex media. (A) Experimental timeline. (B) Quantification of FAD mean lifetime (FAD τₘ) of CAR T cells expanded in either ImmunoCult XF or TexMACS media. n=666-751 cells/condition across 3 independent donors, Mann-Whitney test. (C) Representative image of CAR⁺ T cells identified based on PerCP conjugated GD-2 CAR antibody (grey). (D-F) OMI metabolic profiles of CAR⁺ T cells differed based on expansion media. (E) UMAP based on 13 OMI parameters of CAR⁺ T cells expanded in Imm or Tex media. (F) ROC curves and AUCs of three models based on OMI parameters (Table 2) to classify CAR⁺ T cells by expansion media (Imm vs. Tex); n=494 cells for training (80%), n=123 cells for testing (20%). (G) Quantification of NAD(P)H τₘ from CAR T cells expanded in ImmunoCult XF media supplemented with 500 U/mL IL-2 compared to other cytokine combinations, n=83-129 cells/condition from 2 donors, ANOVA with Kruskal-Wallis test. * p<0.0001.

FIG. 18. CAR+ T cells expanded in Imm and Tex media showed different phenotypes. (A) Experimental timeline. MFIs of (B) CCR7 and (C) CD62L of CAR⁺ T cells expanded in either Imm or Tex media supplemented with different cytokine cocktails, normalized by fluorescence-minus-one (FMO) controls. (n=87,317 cells from 3 independent donors). UMAP based on Euclidean distances of MFIs of 6 surface markers (CD27, CD45RO, CD62L, CD28, CD45RA and CCR7) of CAR⁺ T cells expanded in Imm and Tex media, color coded based on (D) culture media or (E) supplemented cytokines. Dots are CAR⁺ T cells. n=87,317 cells from 3 independent donors. * p<0.0001.

FIG. 19. CAR T cell phenotypes and metabolic profile before in vivo treatment course and in vivo treatment response. (A) Experimental timeline. (B) Phenotypes of CAR+ T cells expanded in Imm, Tex→Imm, and Tex conditions based on expression of 7 surface markers. (C) Extracellular flux analysis of baseline oxygen consumption rate (OCR) and extracellular acidification rate (ECAR) from CD3 T cells that were unactivated (quiescent) or activated and expanded in Imm or Tex→Imm condition. n=9-18 samples/condition Brown-Forsythe and Welch ANOVA test with Dunnette T3 post hoc test for multiple comparisons against Tex→Imm group. (D) Fold change in tumor flux (measured with IVIS imaging) in NSG tumor-bearing mice after CAR T cell treatment. Dash line represents no change in tumor flux (fold change=1). (E) CAR T treatment outcomes on day 17 post treatment. * p<0.0001.

### DETAILED DESCRIPTION

Before the present invention is described in further detail, it is to be understood that the invention is not limited to the particular embodiments described. It is also understood that the terminology used herein is only for the purpose of describing particular embodiments and is not intended to be limiting. The scope of the present invention will be limited only by the claims. As used herein, the singular forms “a”, “an”, and “the” include plural embodiments unless the context clearly dictates otherwise.

Specific structures, devices, and methods relating to monitoring T cells prior to gene editing are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, the term “T cell” refers to cells that are CD45+ and CD3+.

As used herein, “cell size” refers to a measured geometric area of a cell of interest as determined by analyzing an acquired image of the cell of interest.

As used herein, “nucleus size” refers to a measured geometric area of a nucleus of a cell of interest as determined by analyzing an acquired image of the cell of interest.

As used herein, “cytoplasm size” refers to the “cell size” minus the “nucleus size.”

As used herein, the term “memory” includes a non-volatile medium, e.g., a magnetic media or hard disk, optical storage, or flash memory; a volatile medium, such as system memory, e.g., random access memory (RAM) such as DRAM, SRAM, EDO RAM, RAMBUS RAM, DR DRAM, etc.; or an installation medium, such as software media, e.g., a CD-ROM, or floppy disks, on which programs may be stored and/or data communications may be buffered. The term “memory” may also include other types of memory or combinations thereof.

As used herein, the term “FAD” refers to flavin adenine dinucleotide.

As used herein, the term “NAD(P)H” refers to reduced nicotinamide adenine dinucleotide and/or reduced nicotinamide adenine dinucleotide phosphate.

As used herein, the term “processor” may include one or more processors and memories and/or one or more programmable hardware elements. As used herein, the term “processor” is intended to include any of types of processors, CPUs, GPUs, microcontrollers, digital signal processors, or other devices capable of executing software instructions.

As used herein, the term “redox ratio” or “optical redox ratio” refers to a ratio of NAD(P)H fluorescence intensity to FAD fluorescence intensity; a ratio of FAD fluorescence intensity to NAD(P)H fluorescence intensity; a ratio of NAD(P)H fluorescence intensity to any arithmetic combination including FAD fluorescence intensity; or a ratio of FAD fluorescence intensity to any arithmetic combination including NAD(P)H fluorescence intensity. In certain cases, the redox ratio or optical redox ratio refers to a ratio of NAD(P)H fluorescence intensity to the sum of NAD(P)H and FAD fluorescence intensity.

Autofluorescence endpoints include photon counts/intensity and fluorescence lifetimes. The fluorescence lifetime of cells can be a single value, the mean fluorescence lifetime, or compromised from the lifetime values of multiple subspecies with different lifetimes. In this case, multiple lifetimes and lifetime component amplitude values are extracted. Both NAD(P)H and FAD can exist in quenched (short lifetime) and unquenched (long lifetime) configurations; therefore, the fluorescence decays of NAD(P)H and FAD are fit to two components. Generally, NADH and FAD fluorescence lifetime decays are fit to a two component exponential decay, I(t)=α₁e^{-t/τ1}+α₂e^{-t/τ2}+C, where I(t) is the fluorescence intensity as a function of time, t, after the laser pulse, α₁ and α₂ are the fractional contributions of the short and long lifetime components, respectively (i.e., α₁+α₂=1), τ₁ and τ₂ are the short and long lifetime components, respectively, and C accounts for background light. However, the lifetime decay can be fit to more components (in theory any number of components, although practically up to ~5-6) which would allow quantification of additional lifetimes and component amplitudes. By convention, lifetimes t and amplitudes a are numbered from short to long, but this notation could be reversed. A mean lifetime τₘ can be computed from the lifetime components, (τₘ=α₁τ₁+α₂τ₂ . . . ). Fluorescence lifetimes and lifetime component amplitudes can also be approximated from frequency domain data collection and analysis and gated cameras/detectors. For gated detection, α₁ could be approximated by dividing the detected intensity at early time bins by later time bins. Alternatively, fluorescence anisotropy can be measured by polarization-sensitive detection of the autofluorescence, thus identifying free NAD(P)H as the short rotational diffusion time in the range of 100-700 ps.

FAD α₁ refers to the contribution of bound FAD and is the shortest lifetime that is not dominated (i.e., greater than 50%) by instrument response and/or scattering. FAD α₁ is the contribution associated with FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₁ refers to the bound FAD lifetime and is the shortest lifetime that is not dominated (i.e., greater than 50%) by instrument response and/or scattering. FAD τ₁ is the FAD lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

FAD τ₂ refers to the free FAD lifetime and is the longest lifetime that is not dominated (i.e., greater than 50%) by instrument response and/or scattering. FAD τ₂ is the FAD lifetime values from 1000-4000 ps, from 1000-3000 ps, or from 1500-3000 ps. For clarity, a claim herein including features related to a “longest” lifetime cannot be avoided by defining the lifetime values to include a sacrificial shortest lifetime that is dominated by instrument response and/or scattering.

$$FAD {τ}_{m} = {α}_{1} \cdot {τ}_{1} + ( 1 - {α}_{1} ) \cdot {τ}_{2}$$

NAD(P)H α₁ refers to the contribution of free NAD(P)H and is the shortest lifetime that is not dominated (i.e., greater than 50%) by instrument response and/or scattering. NAD(P)H α₁ is the contribution associated with NAD(P)H lifetime values from 50-1500 ps, from 50-1000 ps, or from 50-600 ps. For clarity, a claim herein including features related to a “shortest” lifetime cannot be avoided by defining the lifetime values to include a sacrificial shortest lifetime that is dominated by instrument response and/or scattering.

NAD(P)H τ₁ refers to the free NAD(P)H lifetime and is the shortest lifetime that is not dominated (i.e., greater than 50%) by instrument response and/or scattering. NAD(P)H τ₁ is the NAD(P)H lifetime values from 200-1500 ns, from 200-1000 ns, or from 200-600 ns. For clarity, a claim herein including features related to a “shortest” lifetime cannot be avoided by defining the lifetime values to include a sacrificial shortest lifetime that is dominated by instrument response and/or scattering.

NAD(P)H τ₂ refers to the bound NAD(P)H lifetime and is the longest lifetime that is not dominated (i.e., greater than 50%) by instrument response and/or scattering. NAD(P)H τ₂ is the NAD(P)H lifetime values from 1000-4000 ns, from 1000-3000 ns, or from 1500-3000 ns. For clarity, a claim herein including features related to a “longest” lifetime cannot be avoided by defining the lifetime values to include a sacrificial shortest lifetime that is dominated by instrument response and/or scattering.

$$NAD ( P ) H {τ}_{m} = {α}_{1} \cdot {τ}_{1} + ( 1 - {α}_{1} ) \cdot {τ}_{2}$$

The autofluorescence data used for the disclosed procedures can be acquired in a variety of ways, as would be understood by one having ordinary skill in the spectroscopic arts with knowledge of this disclosure and their own knowledge from the field. For example, the autofluorescence data can be acquired from fluorescence decay data. As another example, the autofluorescence data can be acquired by gating a detector (a camera, for instance) to acquire data at specific times throughout a decay in order to approximate the autofluorescence endpoints described herein. As yet another example, a frequency domain approach can be used to measure and analyze lifetime. Alternatively, fluorescence anisotropy can be measured by polarization-sensitive detection of the autofluorescence, thus identifying free NAD(P)H as the short rotational diffusion time in the range of 100-700 ps. The specific way in which autofluorescence data is acquired is not intended to be limiting to the scope of the present invention, so long as the lifetime information necessary to determine the autofluorescence endpoints necessary for the methods described herein can be suitably measured, estimated, or determined in any fashion.

The disclosed predictions are computed using at least one metabolic indicator derived from the autofluorescence data set (e.g., OMI and/or morphological parameters) for each T cell of the population of T cells as an input. The at least one metabolic indicator can include one or more of the following: NAD(P)H fluorescence intensity; NAD(P)H shortest lifetime amplitude component or NAD(P)H α₁; NAD(P)H mean fluorescence lifetime or NAD(P)H τₘ; NAD(P)H shortest fluorescence lifetime or NAD(P)H τ₁; NAD(P)H second shortest fluorescence lifetime or NAD(P)H τ₂; an optical redox ratio (e.g., NAD(P)H/[NAD(P)H+FAD], see definition above); FAD fluorescence intensity; FAD mean fluorescence lifetime or FAD τₘ, the FAD shortest fluorescence amplitude component or FAD α₁, the FAD shortest fluorescence lifetime component or FAD τ₁, the FAD longest fluorescence lifetime component or FAD τ₂, or a combination thereof. The relative weightings of the at least one metabolic endpoints can vary based on the particular cell type and prediction and are discussed below with respect to more specific aspects. The at least one morphological parameter can include cell/cytoplasm size, as determined from the autofluorescence data.

In some cases, two, three, four, five, six, seven, eight, nine, ten, eleven, twelve, thirteen, fourteen, or more inputs are used.

In some cases, a surprisingly small number of metabolic endpoints and morphological parameters can be used while still achieving an adequate level of classification accuracy, as described below.

The disclosed methods can be performed without the use of a fluorescent label for binding the T cells and/or without immobilizing the T cells.

The various aspects may be described herein in terms of various functional components and processing steps. It should be appreciated that such components and steps may be realized by any number of hardware components configured to perform the specified functions.

### Methods

This disclosure provides a variety of methods. It should be appreciated that various methods are suitable for use with other methods. Similarly, it should be appreciated that various methods are suitable for use with the systems described elsewhere herein. When a feature of the present disclosure is described with respect to a given method, that feature is also expressly contemplated as being useful for the other methods and systems described herein, unless the context clearly dictates otherwise.

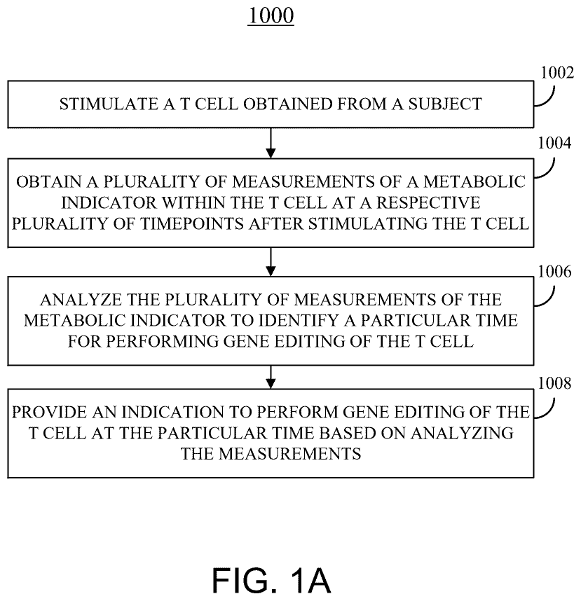

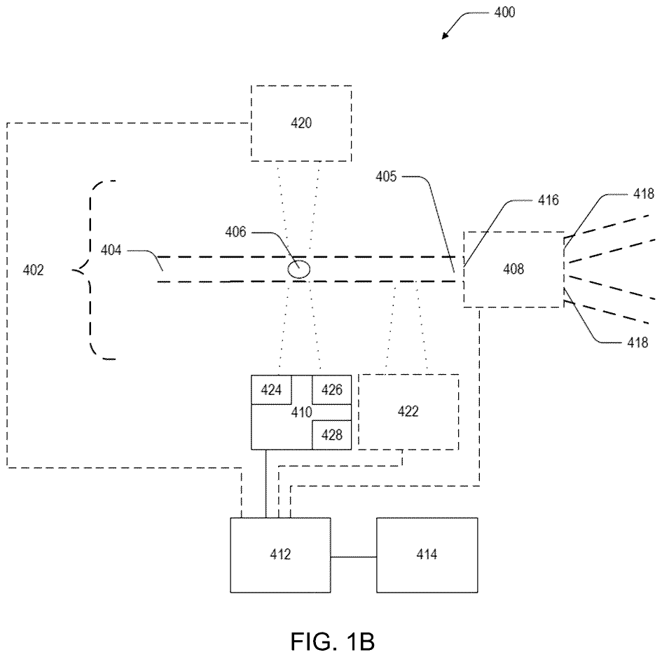

FIG. 1A shows an example 1000 of a process for gene editing of a T cell in accordance with some embodiments of the disclosed subject matter. As shown in FIG. 1A, at 1002, process 1000 can stimulate a T cell obtained from a subject. At 1004, process 1000 can obtain a plurality of measurements of a metabolic indicator within the T cell at a respective plurality of timepoints after stimulating the T cell. At 1006, process 1000 can analyze the plurality of measurements of the metabolic indicator to identify a particular time for performing gene editing of the T cell. Finally, at 1008, process 1000 can provide an indication to perform gene editing of the T cell at the particular time based on analyzing the measurements.

It should be understood that the above-described steps of the process of FIG. 1A can be executed or performed in any order or sequence not limited to the order and sequence shown and described in the figures. Also, some of the above steps of the processes of FIG. 1A can be executed or performed substantially simultaneously where appropriate or in parallel to reduce latency and processing times.

### Systems

This disclosure also provides systems. The systems can be suitable for use with the methods described herein. When a feature of the present disclosure is described with respect to a given system, that feature is also expressly contemplated as being combinable with the other systems and methods described herein, unless the context clearly dictates otherwise.

Referring to FIG. 1B, the present disclosure provides a T cell analysis device 400. The device 400 includes an observation zone 406. The observation zone 406 is adapted to receive a cell analysis pathway 402, a cell culture, or other device or system capable of presenting T cells for optical interrogation. The device 400 includes a processor 412 and a non-transitory computer-readable medium 414, such as a memory. In some configurations, the processor 412 can be or otherwise include a field-programmable gate array (FPGA). In configurations where the processor 412 is an FPGA, an additional processor (not shown) may be included to capture images.

The device 400 optionally includes a cell analysis pathway 402. The cell analysis pathway 402 includes an inlet 404, the observation zone 406, and an outlet 405. The device 400 optionally includes a cell sorter 408. The observation zone 406 is coupled to the inlet 404 downstream of the inlet 404 and is coupled to the outlet 405 upstream of the outlet 405. The device 400 also includes a single-cell autofluorescence spectrometer 410 (which may be a time-resolved autofluorescence decay spectrometer). The device 400 can further include an optional cell picker (not illustrated).

The inlet 404 can be any nanofluidic, microfluidic, or other cell sorting inlet. A person having ordinary skill in the art of fluidics has knowledge of suitable inlets 404 and the present disclosure is not intended to be bound by one specific implementation of an inlet 404.

The outlet can be any nanofluidic, microfluidic, or other cell sorting outlet. A person having ordinary skill in the art of fluidics has knowledge of suitable outlets 405 and the present disclosure is not intended to be bound by one specific implementation of an outlet 405.

The observation zone 406 is configured to present one or more T cells for individual autofluorescence decay interrogation. A person having ordinary skill in the art has knowledge of suitable observation zones 406 and the present disclosure is not intended to be bound by one specific implementation of an observation zone 406.

In various embodiments the observation zone 406 may be part of a cell culture system which may be a closed-loop system that permits T cells to be observed in a non-contact, touch-free manner. The observation zone 406 may be a flat surface such as the surface of a culture dish, a flat-bottomed flask, or other suitable observation chamber.

The optional cell sorter 408 has a sorter inlet 416 and optionally includes two sorter outlets 418. The cell sorter is coupled to the observation zone 406 via the sorter inlet 416 downstream of the observation zone 406. The cell sorter 408 is configured to selectively direct a cell from the sorter inlet 416 to one of the optional two sorter outlets 418 based on a sort signal.

The inlet 404, observation zone 406, outlet 405, and optional cell sorter 408 can be components known to those having ordinary skill in the art to be useful in high-throughput cell screening devices or flow sorters, including commercial flow sorters (e.g., flow cytometers or imaging cytometers). The cell analysis pathway 402 can further optionally include a flow regulator, as would be understood by those having ordinary skill in the art. The flow regulator can be configured to provide flow of cells through the observation zone at a rate that allows the autofluorescence spectrometer 410 to acquire the autofluorescence data set. A useful review of the sorts of fluidics that can be used in combination with the present disclosure is Shields et al., “Microfluidic cell sorting: a review of the advances in the separation of cells from debulking to rare cell isolation,” Lab Chip, 2015 Mar. 7; 15(5): 1230-49, which is incorporated herein by reference in its entirety.

The optional cell picker can serve a similar function as the optional cell sorter 408, namely, isolating cells based on a sort signal. The cell picker can be automated. One example of a suitable cell picker includes an ALS CellCelector™, available commercially from ALS Automated Lab Solutions GmbH, Jena, Germany.

The autofluorescence spectrometer 410 includes a light source 424, a photon-counting detector 426, and photon-counting electronics 428.

The autofluorescence spectrometer 410 can be any spectrometer suitable for acquiring autofluorescence data sets as understood by those having ordinary skill in the optical arts.

Suitable light sources 424 include, but are not limited to, lasers, LEDs, lamps, filtered light, fiber lasers, and the like. The light source 424 can be pulsed, which includes sources that are naturally pulsed and continuous sources that are chopped or otherwise optically modulated with an external component.

The light source 424 can provide pulses of light having a full-width at half maximum (FWHM) pulse width that is of a duration that is adequate to achieve the spectroscopic goals described herein, as would be appreciated by one having ordinary skill in the spectroscopic arts. In some cases, the FWHM pulse width is at least 1 fs, at least 5 fs, at least 10 fs, at least 25 fs, at least 50 fs, at least 100 fs, at least 200 fs, at least 350 fs, at least 500 fs, at least 750 fs, at least 1 ps, at least 3 ps, at least 5 ps, at least 10 ps, at least 20 ps, at least 50 ps, or at least 100 ps. In some cases, the FWHM pulse width is at most 10 ns, at most 1 ns, at most 900 ps, at most 750 ps, at most 600 ps, at most 500 ps, at most 400 ps, at most 250 ps, at most 175 ps, at most 100 ps, at most 75 ps, at most 60 ps, at most 50 ps, at most 35 ps, at most 25 ps, at most 20 ps, at most 15 ps, at most 10 ps, or at most 1 ps.

The light source 424 can emit wavelengths that are tuned to the absorption of NAD(P)H and/or FAD. In some cases, the wavelength is at least 340 nm, at least 345 nm, at least 350 nm, at least 355 nm, at least 360 nm, at least 365 nm, or at least 370 nm. In some cases, the wavelength is at most 415 nm, at most 410 nm, at most 405 nm, at most 400 nm, at most 395 nm, at most 390 nm, at most 385 nm, or at most 380 nm. In some cases, the wavelength is between 360 nm and 415 nm, between 350 nm and 410 nm, or between 370 nm and 380 nm. In some cases, the wavelength is 375 nm. In some cases, the wavelength is 2 times or 3 times these wavelength values (i.e., the frequency is ½ or ⅓). It should be appreciated that pulsed light sources inherently have some degree of bandwidth, so they are never strictly monochromatic. Thus, references herein to “wavelength” refer to either a wavelength at the peak intensity or a weighted average wavelength. In some cases, the pulsed light source 424 is a UV pulsed diode laser. In some cases, the pulsed light source has a wavelength that is double the peak absorption wavelength of NAD(P)H and/or FAD, with an ultrashort pulse duration, such that fluorescence excitation is achieved through two-photon excitation events, as understood by those having ordinary skill in the optical arts. In other embodiments, single-photon excitation may be used, and in still other embodiments other numbers of photons (3, 4, etc.) may be used to provide excitation.

The photon-counting detector 426 can be any detector suitably capable of detecting single photons and delivering an analog or digital output representative of the detected photons. Examples of photon-counting detectors 426 include, but are not limited to, a photomultiplier tube, a photodiode, an avalanche photodiode, a single-photon avalanche diode (SPAD), a charge-coupled device, combinations thereof, and the like.

The photon-counting electronic 428 can include electronics understood by those having ordinary skill in the art to be suitable for use with single-photon detectors 426 to produce the data sets described herein. Examples of suitable photon-counting electronics 428 include, but are not limited to, a field-programmable gate array (FPGA), a dedicated digital signal processor (DSP) with a digitizer and a time-to-digital converter, a time-correlated single photon counting (TCSPC) electronic board with time-to-amplitude and analog-to-digital converter electronics (as implemented by Becker & Hickl, Berlin, Germany), combinations thereof, and the like.

The autofluorescence spectrometer 410 can be directly (i.e., the processor 412 communicates directly with the spectrometer 410 and receives the signals) or indirectly (i.e., the processor 412 communicates with a sub-controller that is specific to the spectrometer 410 and the signals from the spectrometer 410 can be modified or unmodified before sending to the processor 412) controlled by the processor 412. Autofluorescence data sets can be acquired by known spectroscopic methods. Fluorescence lifetime images can also be acquired by known imaging methods and those acquired images can be used by the systems and methods described herein, as would be understood by those having ordinary skill in the spectroscopic arts. The device 400 can include various optical filters tuned to isolate autofluorescence signals of interest. The optical filters can be tuned to the autofluorescence wavelengths of NAD(P)H and/or FAD.

The autofluorescence spectrometer 410 can be configured to acquire the autofluorescence dataset from the detector's 426 electrical output at a repetition rate understood by those having ordinary skill in the spectroscopic arts to be suitable for providing adequate sampling to observe the dynamics disclosed herein. In some cases, the repetition rate can be at least 1 kHz, at least 5 kHz, at least 10 kHz, at least 30 kHz, at least 50 kHz, at least 100 kHz, at least 500 kHz, at least 750 kHz, at least 1 MHz, at least 4 MHz, at least 7 MHz, at least 10 MHz, at least 15 MHz, at least 20 MHz, at least 50 MHz, at least 100 MHz, at least 500 MHz, or at least 1 GHz. In some cases, the repetition rate can be at most 1 THz, at most 800 GHz, at most 500 GHz, at most 250 GHz, at most 150 GHz, at most 100 GHz, at most 70 GHz, at most 50 GHz, at most 25 GHz, at most 15 GHz, at most 10 GHz, at most 6 GHz, at most 2 GHz, at most 1 GHz, at most 750 MHz, at most 500 MHz, at most 400 MHz, at most 250 MHz, at most 175 MHz, or at most 100 MHz. While there can be downside associated with oversampling, in principle the present disclosure can function with as high of a sampling rate as can be achieved with existing technology. The repetition rates identified herein are based on the state of the art at the time the present disclosure was prepared and filed and are not intended to be limiting in the event that future developments facilitate a greater repetition rate.

The pulsed light source 424 can be configured to operate at pulse repetition rates that are adapted to acquire the needed fluorescence lifetime information. The maximum pulse repetition rate is limited by the fluorescence lifetime of the fluorophore of interest. The fluorescence decay must have fully died down by the time the next pulse of light is introduced to the sample in order to avoid ambiguity about the sources of data sets (i.e., to avoid uncertainty as to whether a particular fluorescent photon was initiated by the most recent excitation pulse of light or the one preceding it). The pulsed light source 424 can have a pulse repetition rate of up to 100 MHz, up to 80 MHz, up to 60 MHz, or up to 40 MHz. The lower limit of the pulse repetition rate is more practical in a sense of reducing the overall sampling time, but theoretically the data can be taken very slowly if there is some reason to do so.

The device 400 can optionally include an optical microscope 420 for acquiring visual images of cells that are located in the observation zone 406 or elsewhere along the cell analysis pathway 402.

The device 400 can optionally include a cell size measurement tool 422. The cell size measurement tool 422 can be any device capable of measuring the size of cells, including but not limited to, an optical microscope, such as optical microscope 420. In some cases, the optical microscope and the cell size measurement tool 422 are the same subsystem.

In some cases, the autofluorescence spectrometer 410 and the optical microscope 420 can be integrated into a single optical subsystem. In some cases, the autofluorescence spectrometer 410 and the cell size measurement tool 422 can be integrated into a single optical subsystem. While some aspects of the methods described herein can operate by not utilizing the cell size as an input to the convolutional neural network, it may be useful to measure the cell size for other purposes.

The processor 412 is in electronic communication with the spectrometer 410. The processor 412 is also in electronic communication with, when present, the optional cell sorter 408, the optional optical microscope 420, and the optional cell size measurement tool 422.

The non-transitory computer-readable medium 414 has stored thereon instructions that, when executed by the processor, cause the processor to execute at least a portion of the methods described herein. Equations for which the first and second phasor coordinates are inputs can also be stored on the non-transitory computer-readable medium 414. The non-transitory computer-readable medium 414 can be local to the device 400 or can be remote from the device, so long as it is accessible by the processor 412.

The device 400 can be substantially free of fluorescent labels (i.e., the cell analysis pathway 402 does not include a region for mixing the cell(s) with a fluorescent label). The device 400 can be substantially free of immobilizing agents for binding and immobilizing T cells.

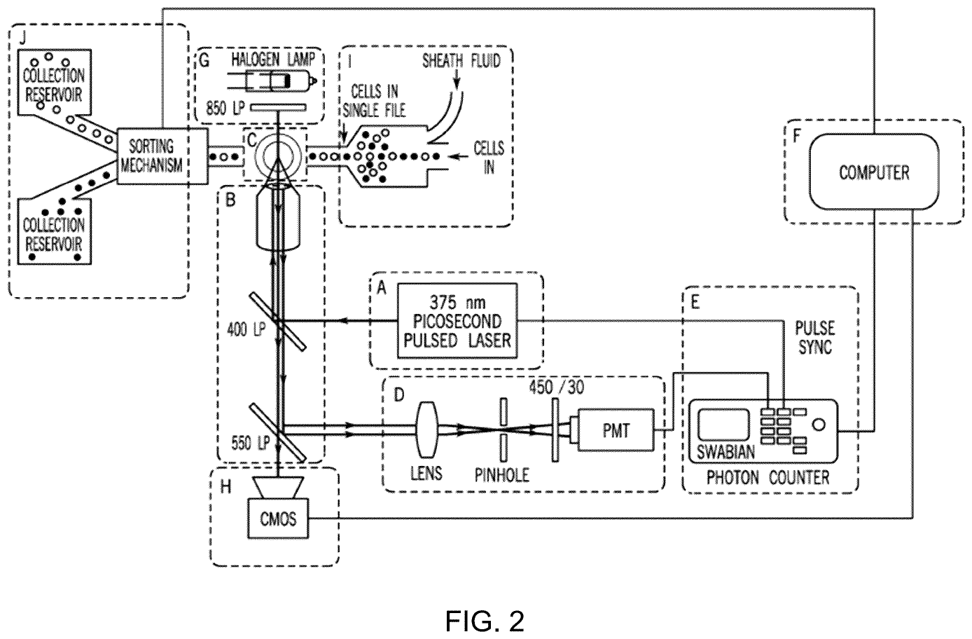

Referring to FIG. 2, one specific implementation of a device in accordance with the present disclosure is illustrated.

FIG. 2 shows the schematic diagram of optical and electronic parts, excitation and emission light paths, and optional fluidic flow and cell sorting mechanisms utilized in the present example. It should be appreciated that this is a non-limiting specific aspect of the present disclosure. While this particular example provides for a fluidic flow and cell sorting mechanism, various embodiments of the disclosed methods and procedures may also be based on non-flow and/or non-sorting based systems (e.g., systems in which cells may be contained in flasks or other containers that are suitable for obtaining optical measurements of the type disclosed herein). In such embodiments, the flow cell interrogation window (component C in FIG. 2) may be the bottom of a dish or flask or other container or compartment.

As with all other methods for time-resolved measurement of fluorescence decay, the decay profile is sampled over many thousands of repeated cycles of excitation followed by detection of emitted decay photons. Therefore, a pulsed excitation light source is used with a temporal pulse width that is much shorter (i.e., by a factor of 100 or more) than the decay lifetime. To measure metabolically-significant NAD(P)H autofluorescence, a pulsed laser at a wavelength of 375 nm is used as the excitation light source. The excitation light travels through the optical light path and illuminates a spot roughly the size of a single immune cell within the field of view of an objective lens focused on the interrogation region of the flow chamber.

The autofluorescence emission travels back through the objective lens and the optical path, and is sent to the detection arm of the system. A focusing lens and a confocal pinhole reject out-of-focus light. The in-focus light that makes it past the pinhole is filtered to only allow the emission spectrum of NAD(P)H autofluorescence (435-465 nm). A photomultiplier tube (PMT) detects emission photons and converts them to an analog electrical signal which is passed to the photon counting electronics.

TCSPC (time-correlated single photon counting) is the standard time-domain fluorescence lifetime measurement method and relies on a time-to-amplitude conversion circuit to register the photon arrival times. Unlike TCSPC, this implementation uses fast FPGA-based (field-programmable gate array) electronics that digitize the analog PMT signal and for every photon event, defined as PMT signal surpassing a user-defined threshold, record a digital time stamp from a fast clock. The digital PMT time stamps are numerically subtracted from similarly acquired laser pulse sync time stamps to build up a histogram of photon arrival times (i.e., the temporal NAD(P)H decay profile) for the immune cell being interrogated.

While the autofluorescence measurement is underway, a high-framerate video camera sensor (either CMOS: complementary metal-oxide-semiconductor or CCD: charge-coupled device) captures a bright-field image of the immune cell under interrogation to provide additional information about shape and size of the cell. The illumination for this imaging is provided by an incandescent microscope lamp (e.g., a halogen lamp) that is filtered to pass only the near-infrared portion of its spectrum, in order to avoid interference with NAD(P)H autofluorescence detection.

In FIG. 2, the system is broken down into functional blocks. Table 1 sets forth the components contained within those functional blocks and identifies commercial sources for these components where appropriate.

TABLE 1
Components of the functional blocks of the system in FIG. 2 and commercial sources.

| Block label | Block description | Manufacturer | Part numbers |
|---|---|---|---|
| A | Pulsed excitation UV laser | Omicron | QuixX ®375-70 PS |
| | source | | |
| B | Optical light path, longpass | Nikon | Nikon Eclipse Ti-S |
| | dichroic mirrors, and objective | Thorlabs | Nikon CFI Super Fluor 40X air |
| | lens | | Nikon Plan Fluor 100x Oil |
| C | Flow cell interrogation window | In house | |
| D | Detection arm: confocal | Thorlabs | P50D-Ø1″ |
| | pinhole, emission filter, and | Hamamatsu | H10721 |
| | PMT | | |
| E | Photon counting and electronics | Swabian | Time Tagger Ultra 8 |
| | | Instruments | |
| F | Computer and display | Dell | |
| G | Bright-field illumination light | Nikon | Nikon D-LF/LC |
| | source and longpass filter | Thorlabs | FGL850S |
| H | CMOS camera for bright-field | Basler | acA1300-200 um Ace |
| | imaging | | |
| I | Microfluidic pump and custom | ElveFlow | OB1 MK3+ |
| | flow cell | | |
| J | Sorting mechanism | In house | |

### Procedures for Predicting Gene Editing Timing

T cell therapies could revolutionize cancer treatment as the first 6 chimeric antigen receptor (CAR) T cell therapies were recently approved and >800 CAR and T cell therapies are in clinical trials. However, barriers remain in achieving durable remissions (>1 year) for approximately half of patients who receive CAR T cell therapy. Due to the rapid development of these therapies and a great need for process optimization, the present disclosure provides improvements to various translational roadblocks to effective CAR T cell therapy, including optimizing in vitro CAR T cell production conditions for higher potency.

Gene editing is required for CAR T cell and other cell therapies, using CRISPR or viral editing. The present inventors have investigated whether the efficiency of gene editing may depend on timing the editing event with cellular events such as cell mitosis.

Accordingly, disclosed herein are embodiments of label-free, touch-free, and non-invasive methods and systems to monitor single cell metabolism to identify an optimal time for performing gene editing. In various embodiments, Optical Metabolic Imaging (OMI) may be used to non-invasively monitor auto-fluorescence signals over time. In some embodiments it has been determined that optimal transfection efficiency is related to cell cycle stage, and in particular embodiments it has been shown that performing transfection during G2/M phase increases gene editing efficiency.

OMI exploits the intrinsic fluorescence of the metabolic co-enzymes FAD and NAD(P)H [NADH and NADPH are optically indistinguishable and referred to collectively as NAD(P)H]. NAD(P)H is an electron donor in hundreds of reactions in the cytosol and mitochondria, while FAD is an electron acceptor mainly in the mitochondria. Therefore, the fluorescence intensity of NAD(P)H divided by that of FAD is the “optical redox ratio”, which provides a label-free measurement of the oxidation-reduction state of a cell. The fluorescence lifetime of NAD(P)H and FAD are distinct in the free and protein-bound conformations, so fluorescence lifetime imaging of these molecules provides information on protein-binding activities, preferred protein-binding partners, and other environmental factors like pH and oxygen. Thus, in various embodiments the disclosed OMI procedures may provide 13 metabolic indicators (Table 2). In further embodiments, cell morphology can also be extracted from autofluorescence data because the signal is localized to the cytoplasm, including 40 parameters based on a single cell segmentation pipeline (e.g., cytoplasm or cell size, cell diameter, cell area, cell circularity (Python)).

As T cell metabolism strongly correlates with function, Optical Metabolic Imaging (OMI) has been used to identify the optimal timeframe for genome editing (e.g., as a function of time following cell activation) to achieve highest genome editing efficiency. OMI allows non-invasive, real-time characterization of cell metabolism based on endogenous autofluorescence from metabolic coenzymes NAD(P)H and FAD and quantifies their relative abundance and binding activity. Autofluorescence data from OMI are processed and used to extract various metabolic indicators (Table 2) based on curve-fitting or fit-free procedures.

TABLE 2
List of OMI parameters (13 metabolic features + cytoplasm size). OMI
parameters are derived from autofluorescence intensity and
fluorescence lifetimes of NAD(P)H and FAD. Each
parameter is measured from the cytoplasmic
region on a single-cell level using manually segmented masks.
List of OMI parameters

| | |
|---|---|
| NAD(P)H intensity | Number of NAD(P)H photons |
| NAD(P)H τ₁ | Fluorescence lifetime of free NAD(P)H |
| NAD(P)H τ₂ | Fluorescence lifetime of bound NAD(P)H |
| NAD(P)H α₁ | Fraction of free NAD(P)H |
| NAD(P)H α₂ | Fraction of bound NAD(P)H |
| NAD(P)H τₘ | Mean NAD(P)H fluorescence lifetime = α₁τ₁ + α₂τ₂ |
| FAD intensity | Number of FAD photons |
| FAD τ₁ | Fluorescence lifetime of bound FAD |
| FAD τ₂ | Fluorescence lifetime of free FAD |
| FAD α₁ | Fraction of bound FAD |
| FAD α₂ | Fraction of free FAD |
| FAD τₘ | Mean FAD fluorescence lifetime = α₁τ₁ + α₁τ₂ |
| Redox ratio | $\frac{NAD ( P ) {H}_{intensity}}{NAD ( P ) {H}_{intensity} + F A {D}_{intensity}}$ |
| Cytoplasm size | Number of pixels in cytoplasm mask |

In contrast to OMI, current methods to evaluate cell metabolism and cell cycle stage are destructive and therefore may be difficult to implement in a cell manufacturing setting. Here, the method provides an in-line monitoring system to rapidly determine optimal timing for gene editing. In various embodiments, these optimized timings can improve the proportion of gene edited cells from ~5% to ~20%, providing meaningful information for final product quality.

In certain embodiments the disclosed methods and systems may be used as a research tool to optimize protocols for CAR T cell production. In various other embodiments, as this industry moves to adaptive processing, these methods may be used in the production of edited T-cells for patient therapies.

In various embodiments, the disclosed methods and systems are capable of identifying the optimal timing for gene editing of T-cells based on one or more factors including OMI measurements of NAD(P)H parameters and cytoplasm and/or cell size, although FAD autofluorescence had less prediction power than the other metrics.

In practice, T cells may be obtained from a subject (e.g., a patient in need of treatment such as treatment with CAR T cells) and may subsequently be stimulated, for example by contacting the cells with an antibody reagent. The antibody reagent can be any reagent for in vitro stimulation, activation and expansion of cells. The antibody reagent can be a small molecule, a polymer, a matrix, or a complex of biomolecules, or a combination thereof. In an aspect, the cells can be contacted with at least one of ImmunoCult™ Human CD3/CD28/CD2 T Cell Activator (αCD2/αCD3/αCD28) (sometimes referred to herein as “ImmunoCult”) or Human T Cell TransAct™ (αCD3/αCD28) (sometimes referred to herein as “TransAct”) antibody reagents.

Following stimulation, OMI and other data may be obtained from one or more of the T cells at the time of stimulation (t=0) as well as at various times thereafter, including one or more times between >0 h and up to 100 h, including 12, 24, 36, 48, 60, or 72 h following stimulation, or at other suitable time intervals. In various embodiments, data may be obtained from multiple T cells (e.g., from 100-10,000 cells) at each timepoint so that the data from the multiple T cells can be averaged or otherwise combined to provide more meaningful data.

In particular embodiments, one or more of the stimulated T cells may be located in an observation zone (e.g. a chamber of a flow system or a flat surface such as a bottom surface of a flask or dish) where optical measurements may be obtained. At a particular timepoint (either at the time of stimulation, t=0, or a later time), optical data may be obtained (e.g., using OMI) from the one or more T cells. As described further herein, the data from one or more T cells at each timepoint may be analyzed to obtain one or more metabolic indicators. The data and resulting metabolic indicators from multiple timepoints may then be analyzed to identify patterns or trends which can be used to identify an optimal time for gene editing of T cells.

For initial studies in this area, groups of T cells may undergo genome editing (which may include transfection or transduction) at different times (e.g., at 12, 24, 36, 48, 60, or 72 h following stimulation), during which the OMI and other data may be collected. After transfection and collection of OMI and other data, the T cells may then be cultured for a period of time (e.g., 7 days) and then harvested and evaluated for the percentage of cells with successful gene editing (% CAR+ cells).

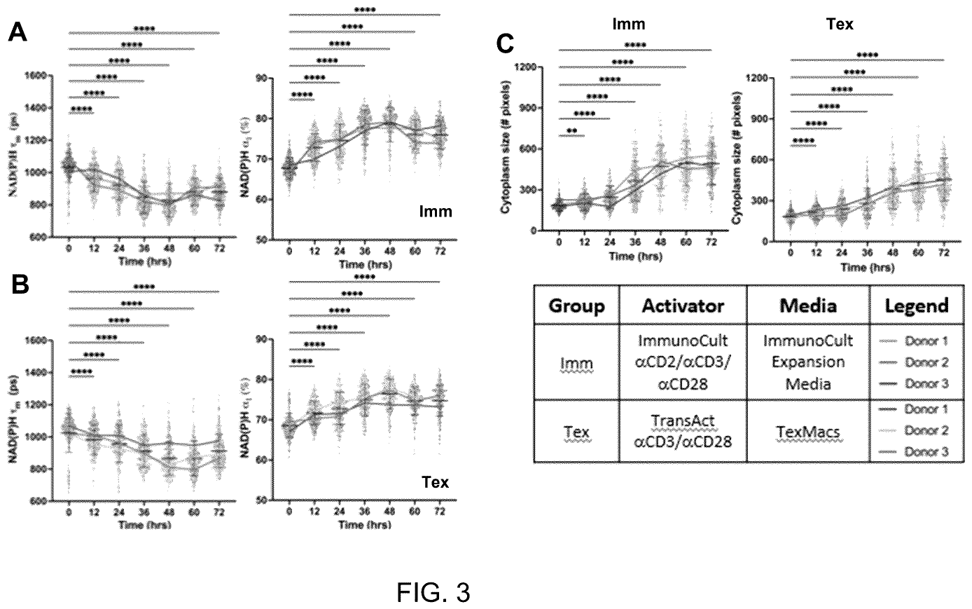

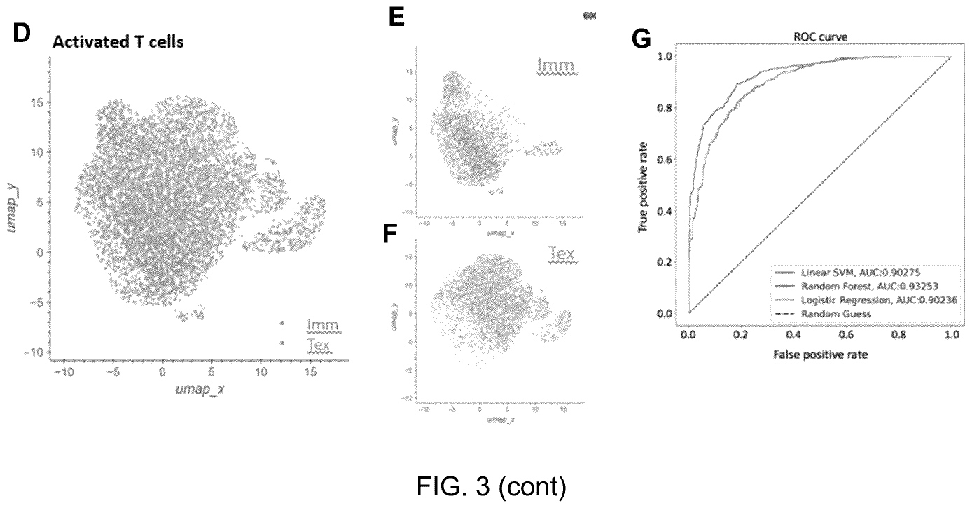

As shown in FIGS. 3-6, the OMI and other data that are collected at the time of transfection can then be processed and analyzed and compared to the percentage of CAR positive cells obtained in each of the respective groups of T cells from the various timepoints. This analysis can shed light on which of the OMI or other parameters or indicators can be used to predict an optimal time for performing gene editing on T cells.

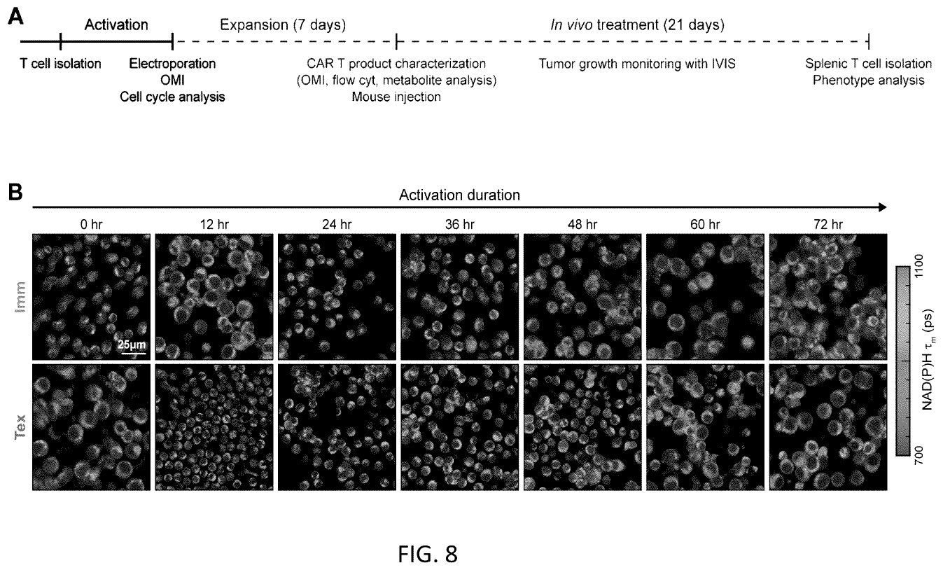

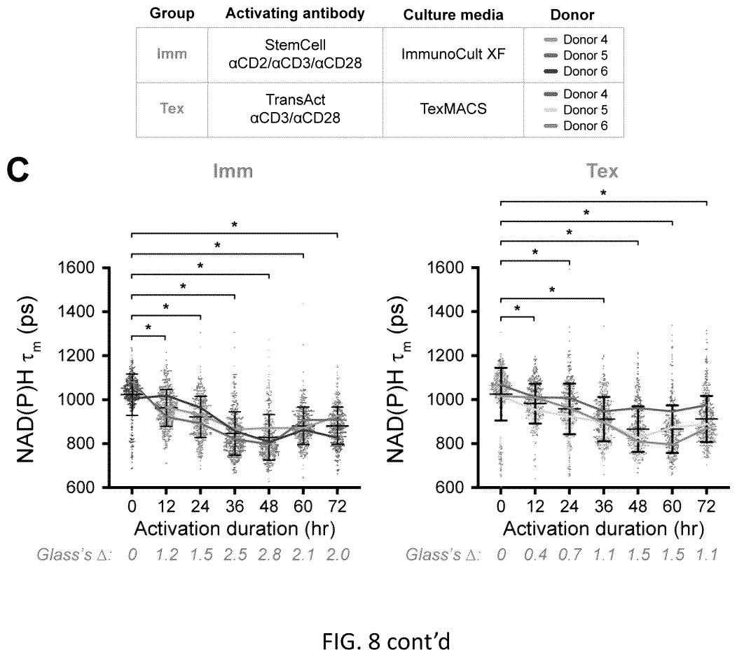

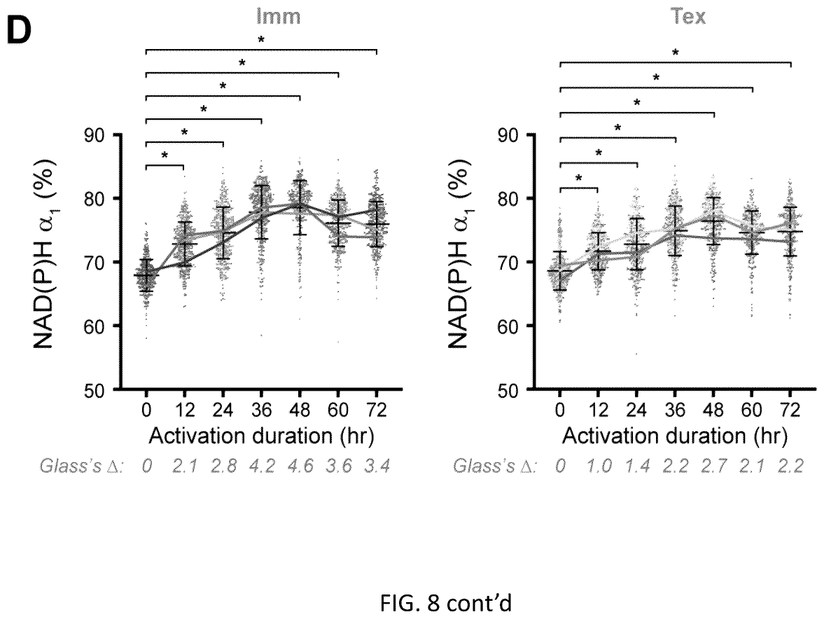

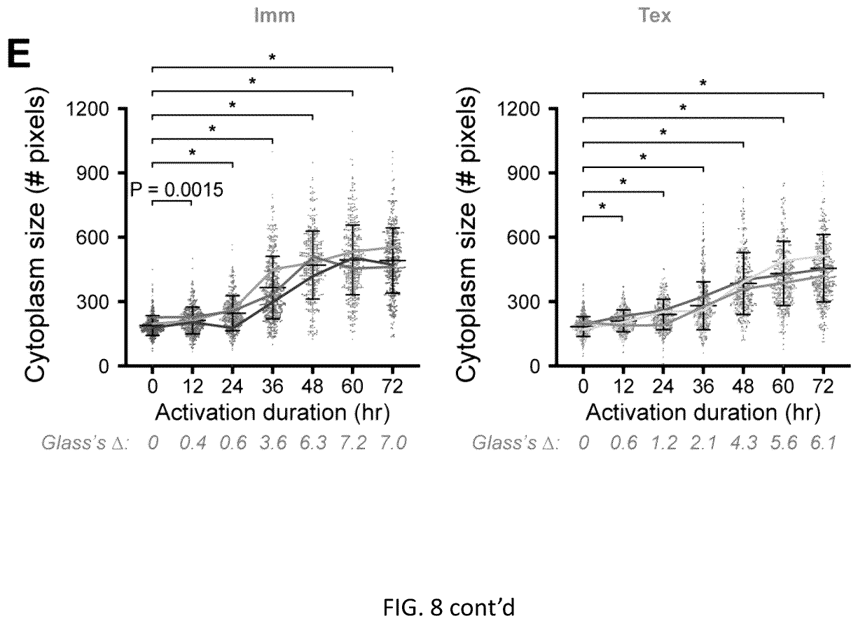

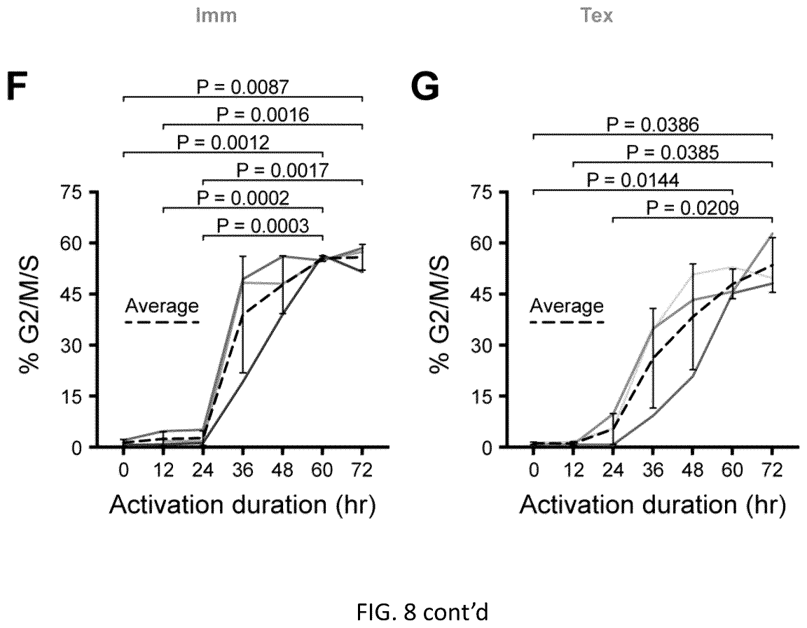

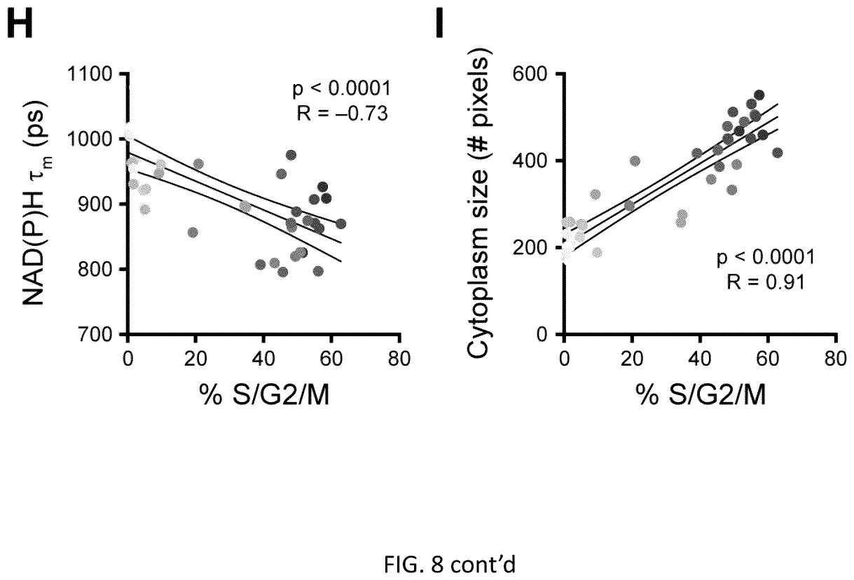

FIG. 3 and FIG. 8 provide additional data showing that OMI can be used to characterize CD3 T cell metabolism and morphology. Compared to quiescent cells, T cells activated with Imm method or Tex method showed significantly (FIG. 3A) lower NAD(P)H τₘ, higher free NAD(P)H fraction (NAD(P)H α₁) (FIG. 3B), and larger cytoplasm size (FIG. 3C). FIGS. 8C-E include Glass's delta values. In FIGS. 3D-3G, T cells subjected to different activation methods showed distinct OMI metabolic profiles. In FIGS. 3E-3G, a Uniform Manifold Approximation Projection (UMAP) based on Euclidean distance of 11 OMI parameters showed distinct clustering of T cells activated with Imm or Tex methods from 3 donors, n=5929 cells. In FIG. 3H, three classification models (Support Vector Machine, Random Forest and Logistic Regression) were trained to classify activated T cells by activation methods (Imm versus Tex) based on the 14 parameters (13 metabolic features (Table 2)+cytoplasm size). Data were randomly split into 80% for training and 20% for testing (n=4743 cells for trainings, 1186 for testing). Receiver operating characteristic (ROC) curve and area under the curve (AUC) were calculated for each classification method, where AUC=1 represents 100% classification accuracy for test data set.

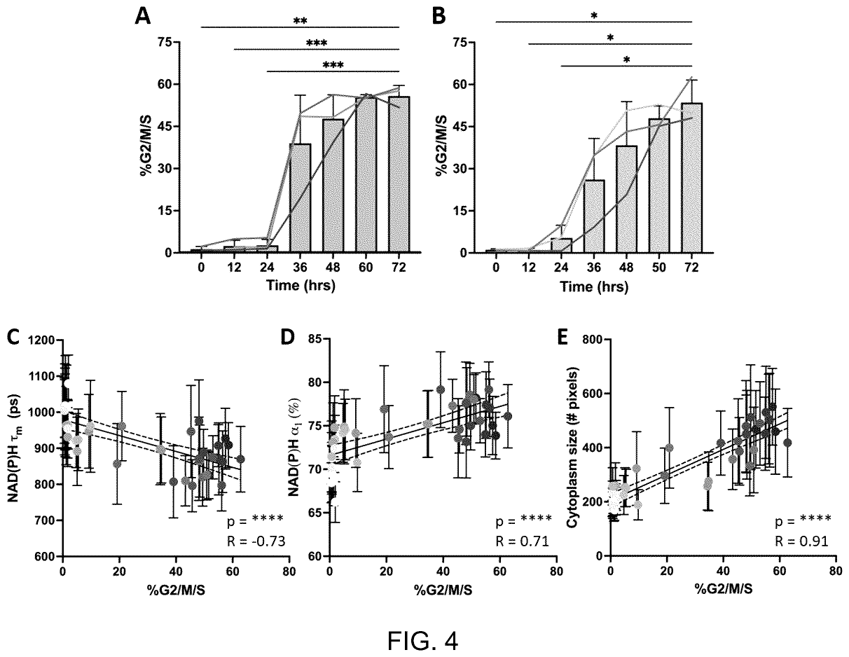

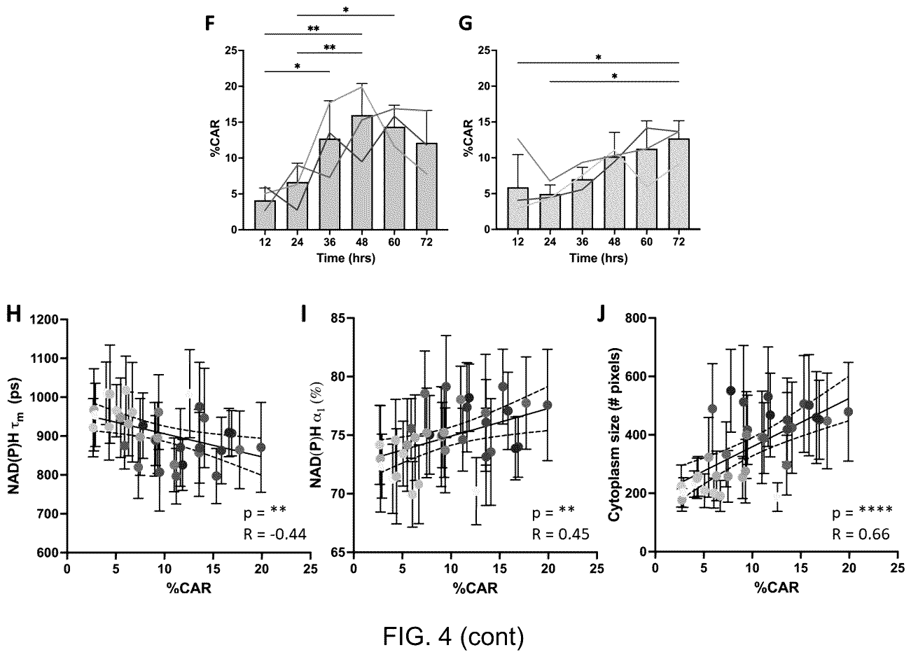

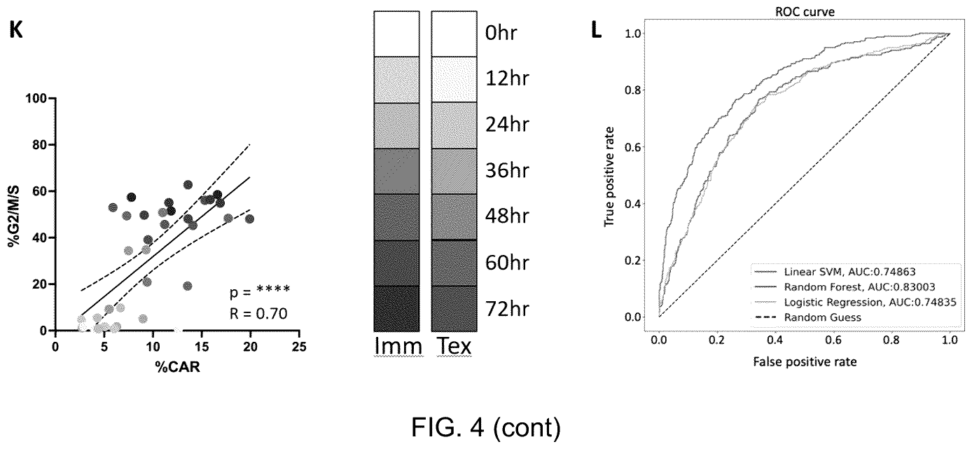

FIGS. 4, 8, and 9 show that OMI was sensitive to metabolic changes as T cells progressed through the cell cycle following activation and allowed identification of an optimal time frame for genome editing via CRISPR/Cas9. T cells activated using the Imm method (FIGS. 4A and 8F with histograms removed for clarity) and the Tex method (FIGS. 4B and 8G with histograms removed for clarity) showed significantly higher proportion of cells in G2/M/S phase at 72 hours compared to earlier timepoints. OMI parameters including NAD(P)H τₘ, (FIGS. 4C and 8H with error bars removed for clarity), NAD(P)H α₁ (FIG. 4D) and cytoplasm size (FIGS. 4E and 8I with error bars removed for clarity), showed significant and strong correlations with the percentage of cells in G2/M/S phase. Each dot represents one sample average, color coded based on the method of activation (Imm versus Tex) and duration of activation (0-72 hours). FIGS. 8F-I are similar to FIGS. 4A-E however they include P values, and error bars have been removed for clarity.

Activation durations and methods affected the gene editing efficiency of the T cells' gene, as quantified by percentage of successful CAR incorporation. Imm activated T cells (FIG. 4F, 9B with histograms removed for clarity) and Tex activated T cells (FIG. 4G, 9C with histograms removed for clarity) showed peak editing efficiency with different activation duration (48-60 hours and 60-72 hours, respectively). OMI measurements, including NAD(P)H τₘ (FIG. 4H, 9E with error bars removed for clarity), NAD(P)H α₁ (FIG. 4I, 16B), and cytoplasm size (FIG. 4J, 9F with error bars removed for clarity), of T cells at each transfection timepoint showed significant and moderate-to-strong correlations to gene editing efficiency (% CAR). Quiescent T cells with 0 hour of activation duration did not undergo transfection. Cell cycle stage (% G2/M/S) at each transfection timepoint showed strong and positive correlation to transfection outcome (% CAR) (FIG. 4K). As shown in FIG. 4L, three classification models were trained on 80% data (n=3881 cells) and tested on 20% data (n=970 cells) to predict gene editing outcomes (high versus low efficiency) based on OMI metabolic and morphological profiles at each transfection timepoint, where 15% CAR for Imm activated T cells and 10% CAR for Tex activated T cells is the threshold for high editing efficiency.

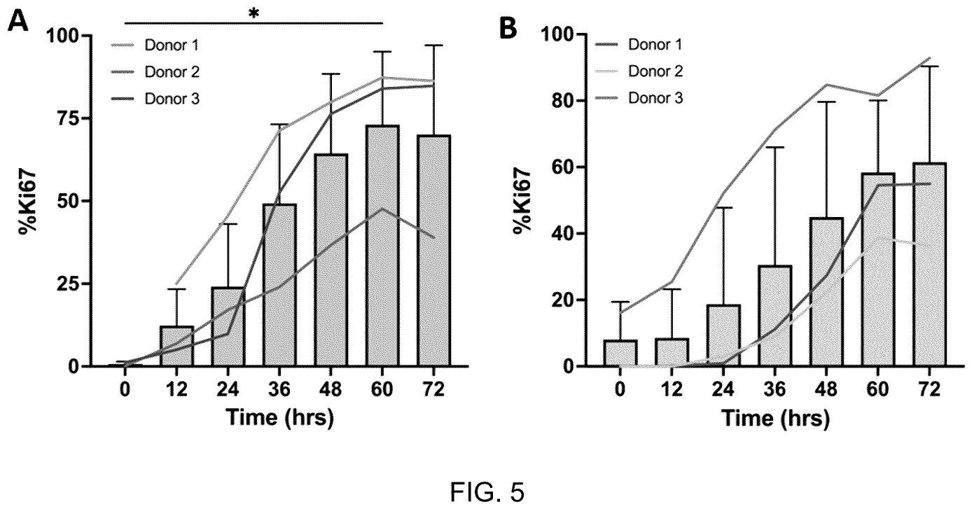

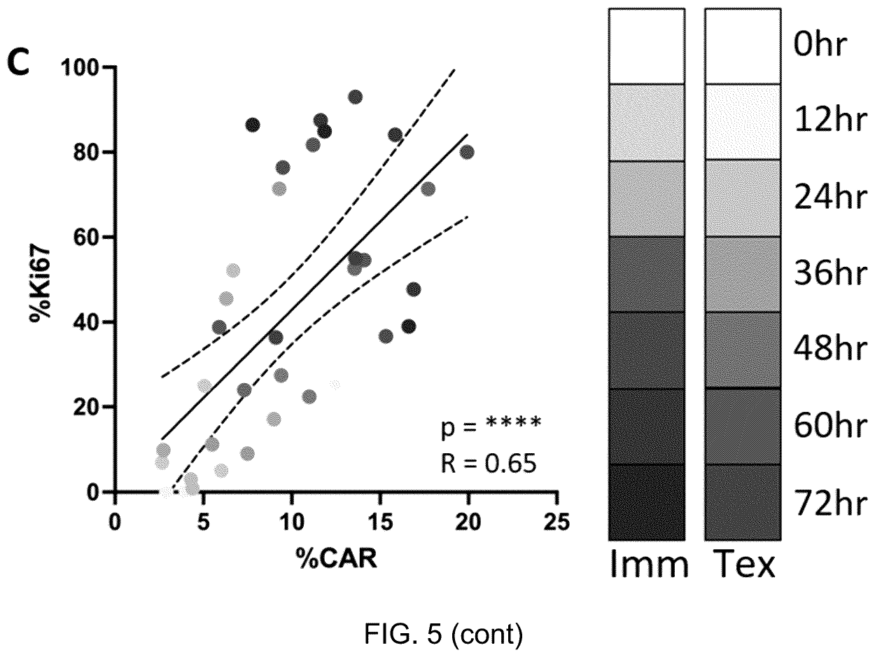

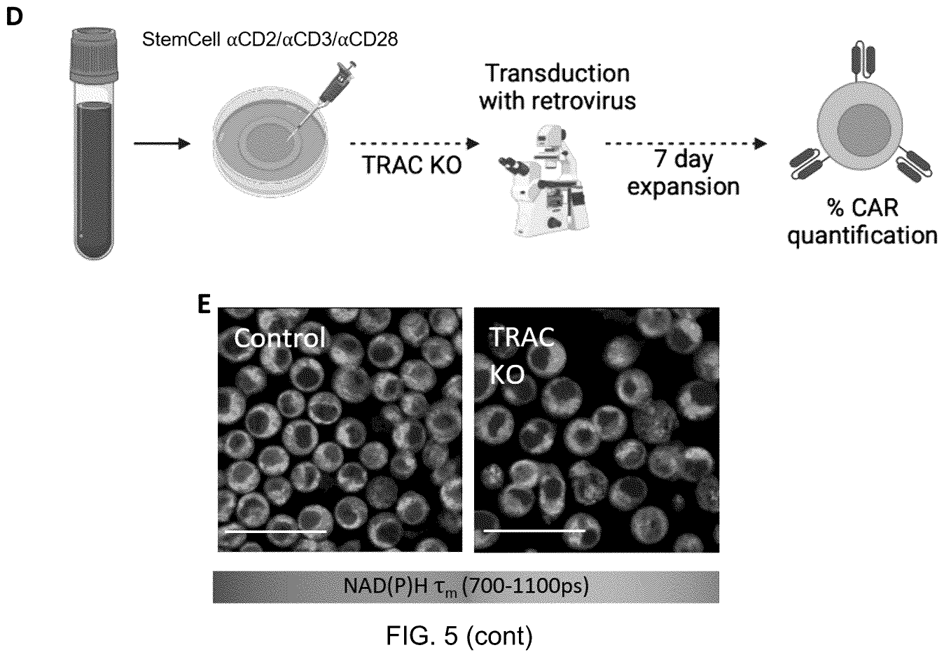

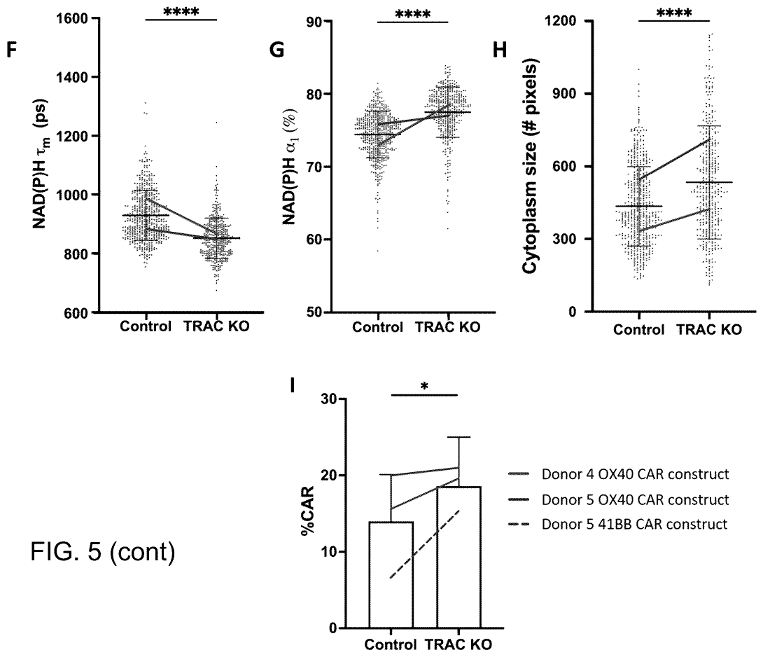

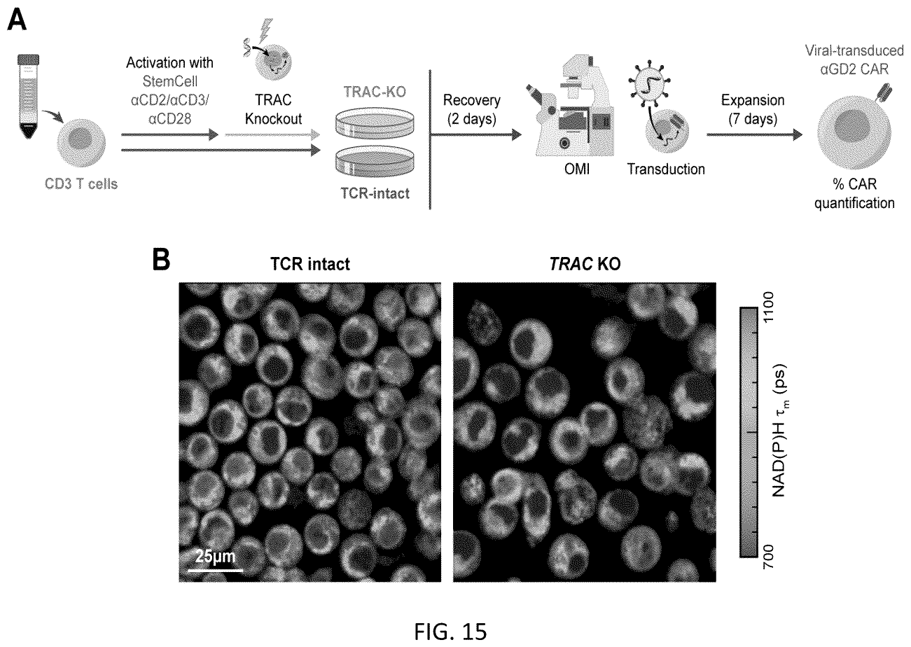

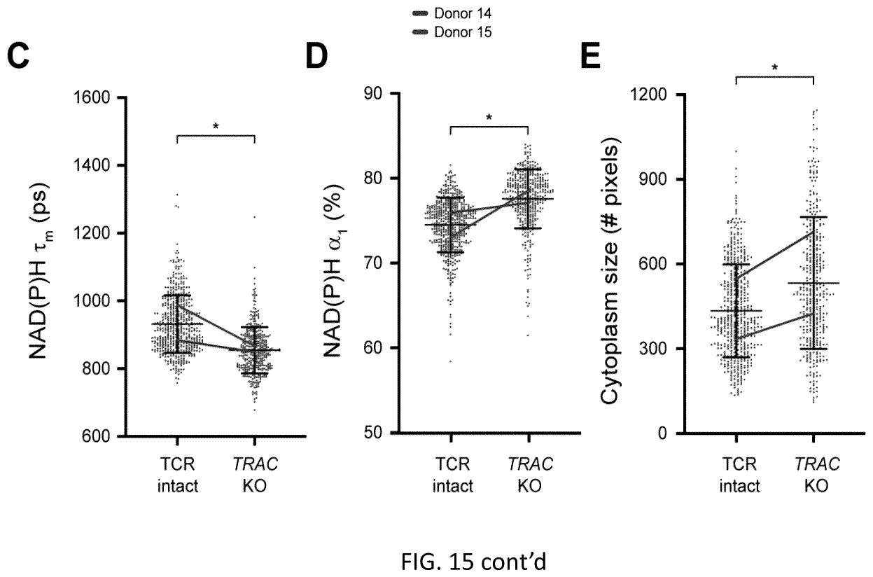

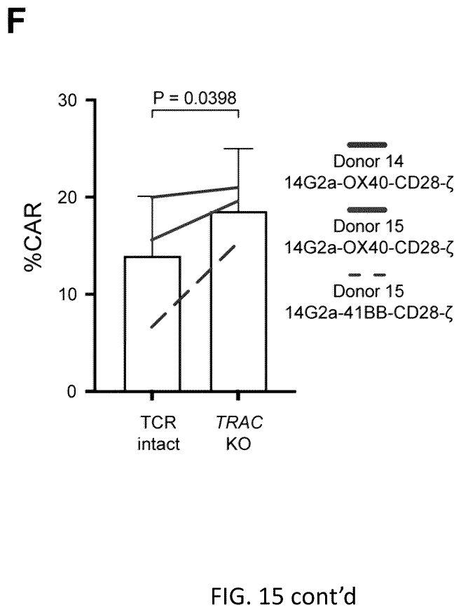

FIGS. 5, 14 and 15 show additional data indicating that timing of gene editing correlates significantly with editing outcomes. Following activation with (FIG. 5A, 14E) Imm method or (FIG. 5B, 14F) Tex method, T cell proliferation increased (% Ki67), and (FIG. 5C) proliferation rate at gene editing time point strongly correlated with gene editing outcome (% CAR). T cells with lower NAD(P)H τₘ, higher NAD(P)H α₁ and larger cytoplasm size at viral transduction time point showed significantly higher transduction efficiency. FIG. 5D shows the experimental setup and FIG. 15A shows the experimental timeline. T cells were isolated from peripheral blood of two healthy donor and activated with Imm method and underwent TCR (TRAC) knockout prior to transduction with retroviral to express GD-2 CAR receptor with two constructs (OX40-CD28-CAR or 41BB-CAR) following previously described methods. FIGS. 5E and 15B show representative NAD(P)H τₘ images of control and TRAC KO T cells at transduction. OMI measurements showed that TRAC KO T cells had significantly (FIGS. 5F, 15C) lower NAD(P)H τₘ, (FIG. 5G, 15D) higher NAD(P)H α₁ and (FIGS. 5H, 15E) greater cytoplasm size at transduction. FIGS. 5I, 15F show that, compared to control cells, TRAC KO T cells yielded significantly higher transduction efficiency, quantified as the percentage of CAR+ T cells at harvest.

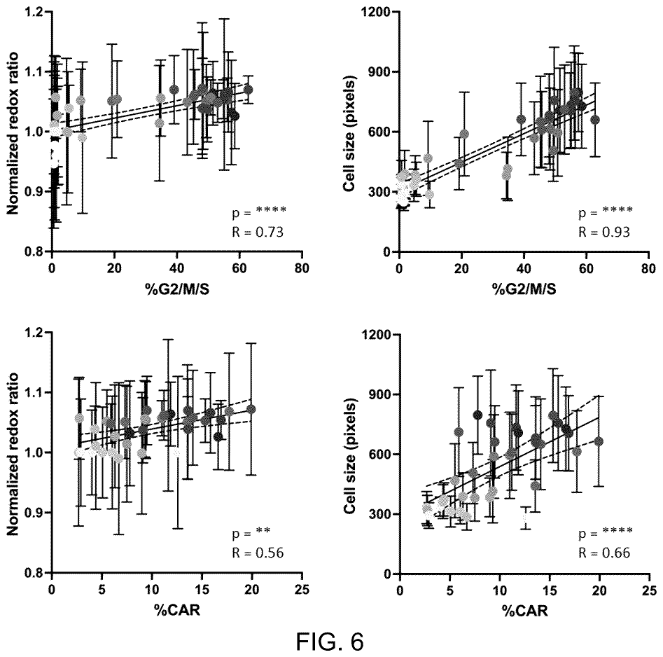

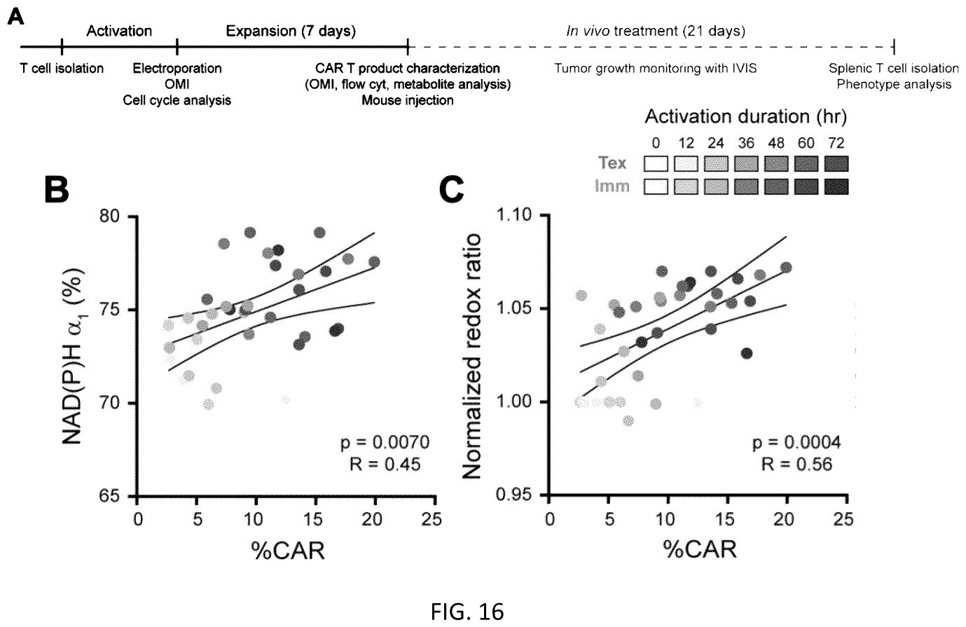

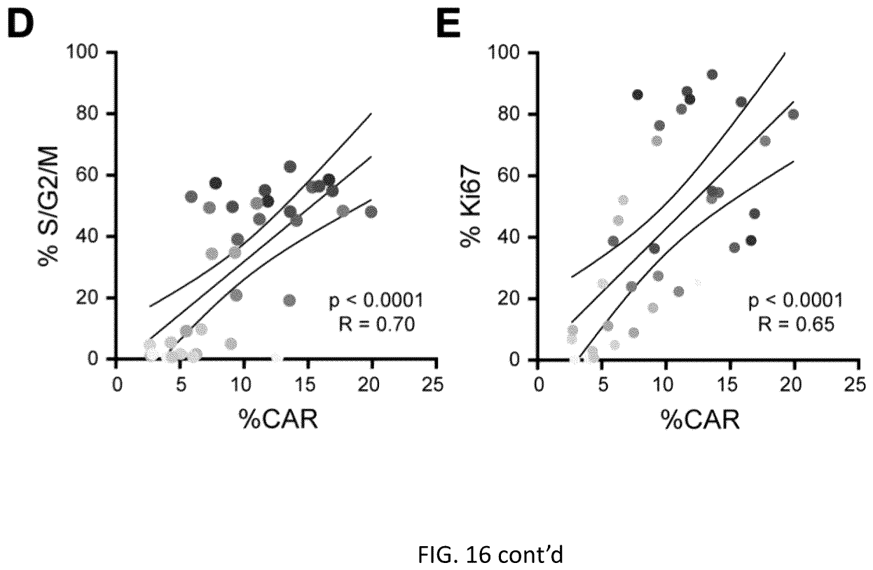

FIG. 6 shows correlations between normalized redox ratio and whole cell size compared to % G2/M/S and % CAR. For each donor and activation method, redox ratios at different activation durations were normalized to donor-match T cells activated for 12 hours of the corresponding activation method (Tex or Imm). The data show significant and strong (% G2/M/S vs. cell size) and moderate correlations (% CAR vs. cell size), respectively (n=40 for % G2/M/S correlation and n=35 for % CAR correlation). Thus, along with other parameters including cytoplasm size, cell size is also predictive of an optimal time for gene editing. FIG. 16C also shows the correlation of redox ratio and % CAR with the error bars removed for clarity.

Overall, this data shows that OMI and other parameters or indicators are predictive of an optimal time for performing gene editing in T cells following stimulation. In particular, the analysis shows that an optimal time for performing gene editing of T cells corresponds to when NAD(P)H τₘ reaches a minimum and/or when NAD(P)H α₁ and/or cytoplasm size and/or cell size reach a maximum.

The data also show that the cell cycle state of the T cells may be predictive of an optimal time for gene editing, with the timepoints which correspond to when the T cells are at the G2/M transition signaling an optimal time frame for performing gene editing. Thus, in various embodiments the present disclosure encompasses methods and systems for performing gene editing based on identifying a cell cycle state of the stimulated T cells and performing gene editing when the cells (or a relatively high percentage of the cells) are at or near the G2/M stage of the cell cycle. Various procedures may be used to determine the cell cycle phase of one or more T cells, including the measurement of cell size and/or cytoplasm size (e.g. using autofluorescence measurements as disclosed herein). In various embodiments, the cell cycle phases of T cells are determined in a label-free and non-destructive manner. Cell cycle phase can also be measured in a non-destructive and label-free manner using other imaging measurements including brightfield imaging, darkfield imaging, quantitative phase imaging, interferometric imaging, differential interference contrast (DIC), digital holography, or Raman microscopy. Cell cycle phase can also be measured in a non-destructive and label-free manner using non-imaging methods including side-scatter, angular scattering, Raman spectroscopy, cell mass, or cellular impedance.

In use (e.g., in laboratory or production settings), the disclosed methods and systems may be used to monitor one or more T cells following stimulation to identify an optimal time for performing gene editing (for example using CRISPR or viral editing procedures). Upon identification of a measurement of a particular metabolic indicator which signifies an optimal time for performing gene editing, the disclosed methods and systems can provide an indication (e.g., send a message or provide some other indication either internally or to an outside source) to initiate gene editing.

In various embodiments, an optimal time for gene editing of T cells may be a range of times such as a multi-hour window of time. For example, in some embodiments the optimal time may be a range between approximately 36-48 h after stimulation (e.g., when stimulating with αCD2/αCD3/αCD28) or may be a range between approximately 60-72 h after stimulation (e.g., when stimulating with αCD3/αCD28). Given that the disclosed analysis procedures can be performed in real time or near-real time and also given that the optimal time for performing gene editing may encompass several hours, the disclosed methods and systems can provide an indication that a group of cells has reached an optimal time for gene editing during the course of normal operation.

While the disclosed procedures may be used to continually monitor all cells in a facility, in various embodiments the disclosed methods and systems may also be used to optimize conditions for cell processing, for example during development of a new cell handling system, when working with new batches of cells or reagents, or when changing other parameters in a system. In some embodiments, it may not be necessary to monitor all batches of cells to determine when they have reached an optimal time for gene editing, provided that conditions between various batches have been consistently maintained.

An advantage of the disclosed methods and systems over current procedures is that the present methods and systems are label-free and as such do not require the addition of any exogenous compounds, since the procedures rely on autofluorescence of naturally-occurring molecules (e.g., NAD(P)H and/or FAD). Further, the disclosed procedures also have the advantage of being non-contact and non-destructive so that there is no chance of contamination of the T cells and no loss of sample that is caused by the monitoring procedures.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

### EXAMPLES

### Example 1

### Abstract

Chimeric antigen receptor (CAR) T cell therapy for solid tumors is challenging not only because of the immunosuppressive tumor microenvironment, but also because of a complex manufacturing process that is difficult to monitor. Manufacturing directly impacts CAR T cell yield, phenotype, and metabolism, which correlate with in vivo potency and persistence. In particular, though metabolic fitness is a relevant quality attribute, how T cell metabolic requirements vary throughout manufacturing remains unexplored. Here, this limitation is addressed with optical metabolic imaging (OMI), a non-invasive, label-free method to evaluate single-cell metabolism based on autofluorescent metabolic coenzymes NAD(P)H and FAD. Using OMI, the dominating impacts of media composition over the selection of antibody stimulation and/or cytokines on anti-GD2 CAR T cell metabolism, activation strength and kinetics, and phenotype are identified. OMI parameters are demonstrated to indicate cell cycle stage and optimal gene transfer conditions for both viral transduction and electroporation-based CRISPR/Cas9. Notably, in a virus-free CRISPR-edited anti-GD2 CAR T cell model, OMI measurements allowed accurate prediction of an oxidative metabolic phenotype that yielded higher in vivo potency against neuroblastoma. This data supports OMI's potential as a robust, sensitive analytical tool to identify optimal manufacturing conditions and monitor cell metabolism throughout manufacturing for increased CAR T cell yield and metabolic fitness.

### Introduction

Chimeric antigen receptor (CAR) T cells are genetically engineered to target tumor-associated antigens and perform cytotoxic functions to eliminate cancer cells. CAR T cell therapy has changed the approach and outlook for cancer care across several hematological malignancies, with patients from early trials maintaining decade-long remission (1). Despite the anticipation about its application to solid tumors, the translation of CAR T cell therapy on that front has yet to succeed (2). That is in part because the successful clinical translation of CAR T cells for solid tumors involves a complex manufacturing process, each step of which requires optimization to enhance CAR T functions against the immunosuppressive tumor and tumor microenvironment. The general CAR T manufacturing workflow includes: (1) activation of T cells within a leukapheresis product from cancer patients using antibody stimulation, (2) introduction of the CAR transgene, and (3) expansion of the resultant CAR T cells to reach sufficient dosage (3). Much remains to be understood about how to best monitor and thus optimize this process.

First, antibody activation is critical to prime T cells for proliferation and genome editing. Though the effects of certain culture media and stimulating antibodies on T cell expansion and phenotype have been characterized (4, 5), their synergistic impacts on CAR T cell metabolism, functions, and transgene incorporation efficiency have yet to be explored. This knowledge gap makes the selection for optimal activation conditions challenging. Second, while viral transduction is the common CAR gene transfer method used in all six FDA-approved products, it suffers from batch-to-batch variability (6, 7) and safety concerns due to random transgene insertion (8). Meanwhile, despite promising in vivo potency (9, 10), CRISPR-edited CAR T cells face challenges such as low transgene incorporation efficiency and viability (11). Extended ex vivo culture is thus helpful to reach the desired CAR T dosage, but comes with the risk of inducing terminal differentiation that decreases potency (12, 13) while delaying patient treatments. Considering remarkable metabolic changes throughout the cell cycle, T cell metabolic states following activation can potentially indicate cell cycle stage and, thus, the optimal CAR gene transfer timeframe (11, 14, 15). Identifying this optimal time for CAR gene transfer could maximize CAR transgene incorporation efficiency and, hence, minimize the expansion time to reach the desirable dosages. This not only limits T cell differentiation to enhance CAR T potency but also shortens treatment wait-time and increases patient access. Third, similar to media composition and stimulating antibodies used during activation, media composition and cytokines used during expansion can impact CAR T cell phenotype, fitness, and clinical outcomes (16). Several cytokine cocktails, including IL-7 and IL-15, have been studied to control CAR T cell differentiation and retain stem-like characteristics (e.g., expression of CCR7, CD62L, CR45RA) for in vivo potency and persistence (17, 18, 19). Similarly, metabolites in culture media such as glucose and glutamine could condition CAR T cell metabolism and program their epigenetics for enhanced in vivo potency and persistence (16, 18), though their specific contributions at different stages throughout manufacturing have yet to be characterized.

T cell metabolic needs vary throughout manufacturing due to distinct functional demands at each stage. For instance, during activation, metabolic reprogramming is necessary to initiate cell cycle entry and enhance homology-directed repair efficiency for transgene incorporation, Meanwhile, during expansion, fine-tuning T cell metabolism is helpful to support proliferation while minimizing terminal differentiation and exhaustion. Several studies suggest that metabolic conditioning of T cells during manufacturing enables in vivo adaptation to the immunosuppressive tumor microenvironment, which is characterized by high metabolic stress, nutrient scarcity, and toxic metabolic waste. Metabolic fitness—as indicated by features like high dependence on oxidative phosphorylation and fatty acid oxidation, low glycolytic activity, and high mitochondrial mass—has recently emerged as a quality attribute for CAR T cells (20-23). In sum, metabolism plays an intricate role in T cell activation, cell cycle progression, gene transfer efficiency, and in vivo potency. Thus, monitoring T cell metabolism during CAR T manufacturing could enable fine-tuning of manufacturing conditions to match the dynamic metabolic demands of T cells at different phases.

However, the integration of standard metabolic assays into a real-time, non-invasive, adaptive CAR T manufacturing workflow remains challenging. These methods often lack single-cell resolution to characterize heterogeneity within the bulk T cell product (e.g., metabolite quantification from media or extracellular flux analysis), involve manipulation of the CAR T product, or are time consuming (e.g., single-cell metabolomics or flow cytometry). By contrast, optical metabolic imaging (OMI) is a label-free, noninvasive method to characterize metabolism within single cells (24, 25). OMI includes metabolic parameters based on autofluorescence intensities and lifetimes of NAD(P)H and FAD, two metabolic coenzymes that are involved in hundreds of reactions in the mitochondria and cytosol (Table 2). Since reduced NAD(P)H and oxidized FAD are autofluorescent, the optical redox ratio (Table 2), can be used to measure cellular redox balance (26-28). Meanwhile, fluorescence lifetimes indicate the binding activity of these molecules. Due to conformational changes upon protein binding, free NAD(P)H self-quenches and has a short lifetime, while protein-bound NAD(P)H has an extended conformation and hence, longer lifetime. FAD displays an opposite trend, with free and protein-bound FAD having a long and short lifetime, respectively (27-29). OMI provides single-cell resolution to characterize heterogeneity within population and identify cell subsets (24, 30), while offering non-invasive, label-free, and real-time readouts of cell metabolism. Previously, it has been demonstrated that OMI measurements allow label free classification of T cell activation; however, OMI's applications for CAR T manufacturing have yet to be explored.

Here, it is tested whether OMI measurements can inform manufacturing conditions that improve gene transfer efficiency and metabolic fitness of CRISPR-edited anti-GD2 CAR T cells for neuroblastoma treatment. Neuroblastoma is the most common extracranial solid tumor in children with a five-year survival rate of less than 50% in high-risk patients (31, 32). Anti-GD2 CAR T cells are a promising treatment due to ubiquitous overexpression of the disialoganglioside GD2 on neuroblastoma cells (32, 33); however, poor T cell persistence and potency remains a substantial roadblock to clinical success. Using OMI, metabolic changes in T cells upon activation in various conditions were characterized and determined the relationship between T cell metabolism, cell cycle progression, and CAR gene transfer efficiency. OMI was also performed to determine the synergistic impacts of culture media and cytokine cocktails on CAR T cell metabolism and phenotype. Finally, OMI parameters were used to predict the expansion condition that achieved high in vivo potency in a NOD/SCID/IL2Rγc^{-/-} mouse xenograft model of human neuroblastoma. Overall, the results demonstrate and validate a novel method to improve CAR T cell yield and efficacy, while highlighting the implication of T cell metabolism for successful CAR T clinical translation.

### Results

### OMI Reveals that Media Composition Determines T Cell Metabolism and Activation Kinetics

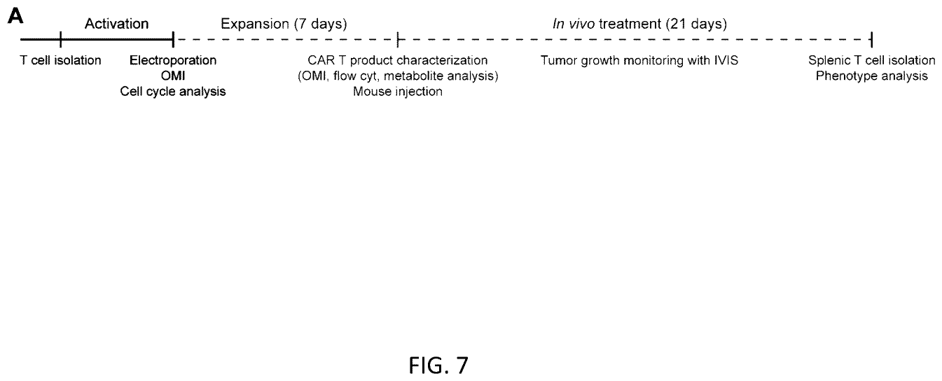

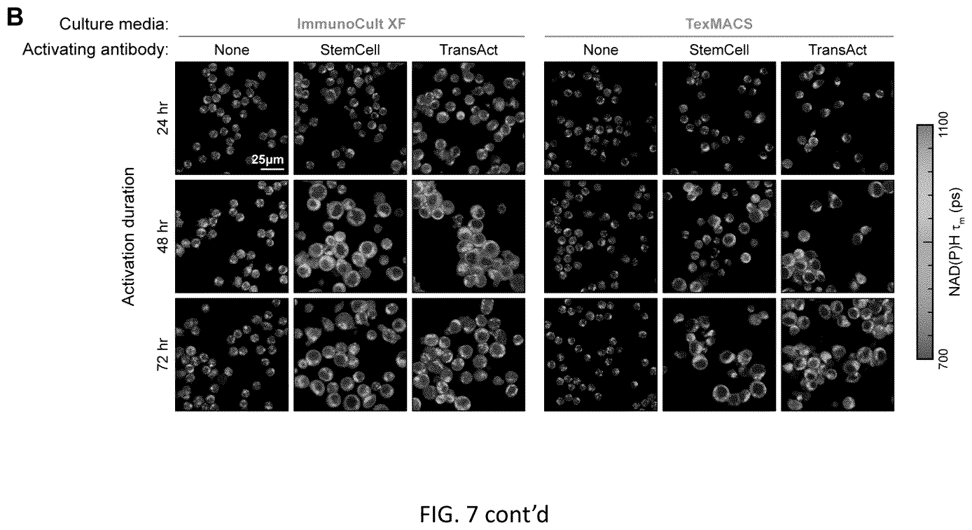

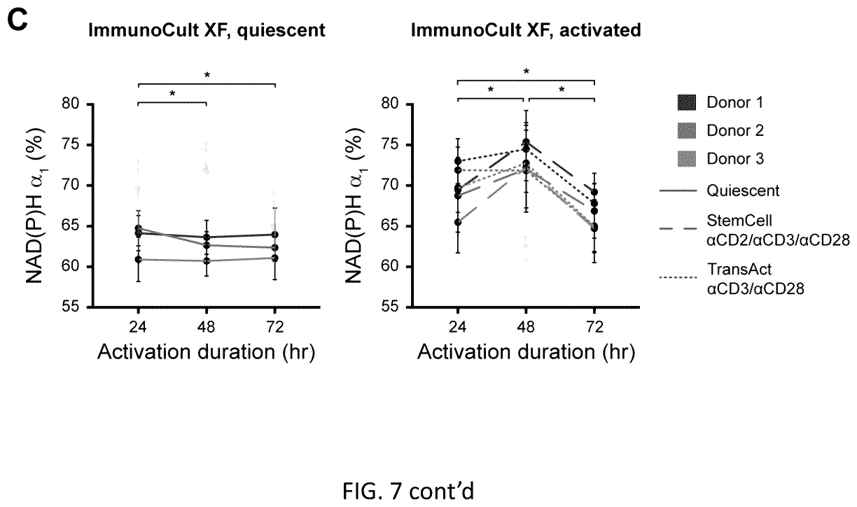

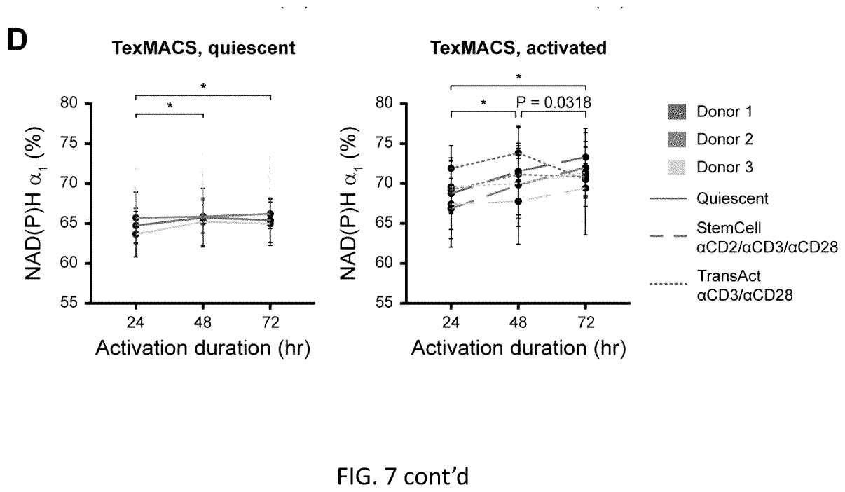

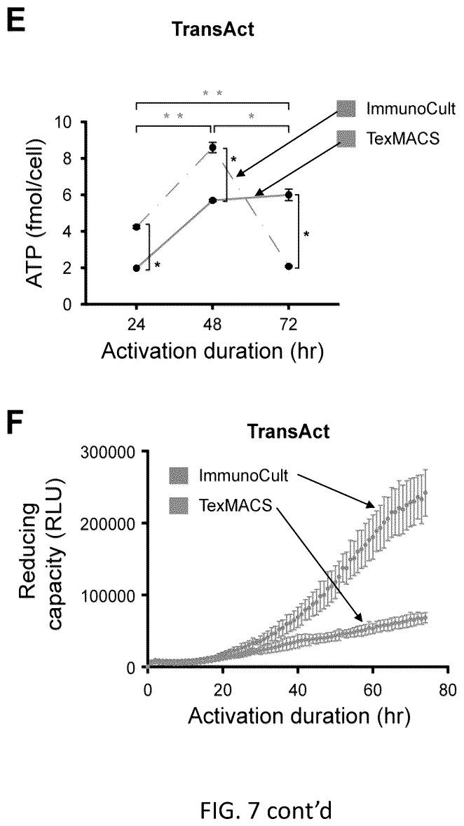

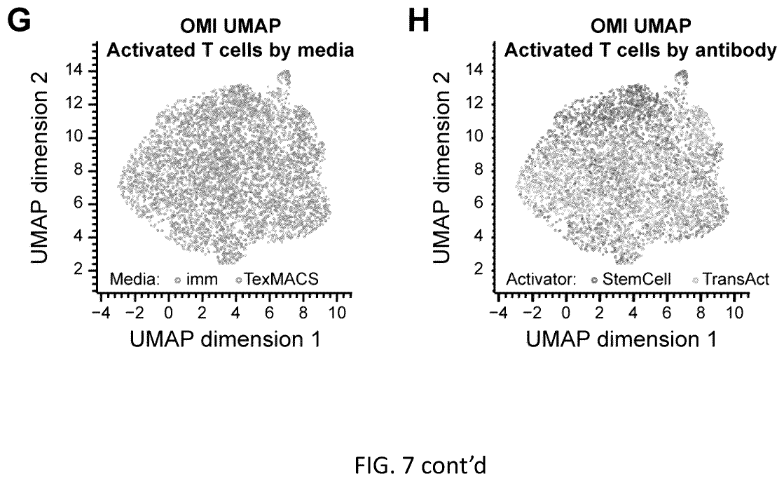

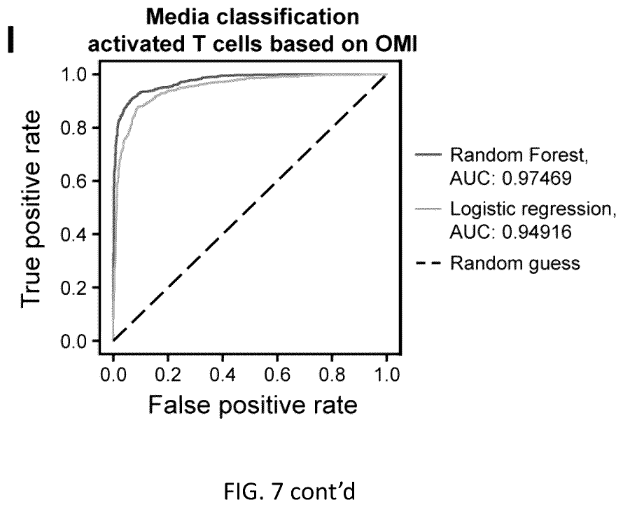

Several media formulations with different concentrations of particular nutrients are used for CAR T production in preclinical and clinical settings (34). Among the most common are ImmunoCult XF media, which has high glucose and glutamine levels, and TexMACS media, which relies on GlutaMax as the source for glutamine and has lower glucose level. Additionally, various soluble T cell activation antibodies have also been developed with different structures and compositions to control activation efficiency, such as StemCell αCD2/αCD3/αCD28 and TransAct αCD3/αCD28. Using OMI, changes in T cell metabolism under various activation conditions were characterized. T cells were activated by four unique combinations of the two antibodies and two media above for 24, 48, and 72 hours and imaged with OMI (FIG. 7A, B). Quiescent T cells cultured in ImmunoCult XF or TexMACS media were also imaged at 24, 48, and 72 hours as the control for activation effect. At each time point, activated T cells showed lower NAD(P)H mean lifetime (NAD(P)H τₘ), higher proportion of free NAD(P)H (NAD(P)H α₁), and increased cell size compared to quiescent cells (FIG. 7B, C). High NAD(P)H α₁ and low NAD(P)H τₘ have been correlated with increased glycolysis (35), suggesting that T cells undergo metabolic shift towards glycolysis upon activation, consistent with prior metabolic studies (36). Consistently across 3 donors, T cells activated in ImmunoCult XF media displayed greatest increase in the NAD(P)H α₁ at 48 hours post activation, regardless of the activating antibodies used (FIG. 7C, right). Meanwhile, T cells activated in TexMACS media demonstrated peak increase in NAD(P)H α₁ at 72 hours post activation for both StemCell and TransAct activated groups (FIG. 7D, right). This suggests that media composition, not activating antibodies, determines the kinetics of metabolic changes in activated T cells.

Metabolic reprogramming upon activation is crucial to balance the energy demands and biosynthesis that supports T cell proliferation and effector functions (37). Thus, ATP production and cellular reducing potential of activated T cells were assessed via bioluminescence-based assay to determine the impact of activation conditions on T cell function and their readiness to proliferate. Early after activation, T cells in glucose^{high}/glutamine^{high} ImmunoCult XF media exhibited significantly higher intracellular ATP levels, peaking at 48 hours, compared to those in glucose^{low}/glutamine^{low} TexMACS media. However, this trend reversed at 72 hours post activation, when T cells activated in TexMACS reached peak ATP production and displayed significantly higher intracellular ATP levels than those in ImmunoCult XF (FIG. 7E). These changes in ATP production are consistent with the NAD(P)H α₁ kinetics as measured by OMI (FIG. 7D). Cellular reducing capacity, an indicator of the overall bioreactivity of intracellular NAD(P)H-dependent reductase enzymes, was measured to further investigate NAD(P)⁺ reducing metabolic pathways (such as glycolysis, pyruvate conversion to acetyl-CoA, or the tricarboxylic cycle) (38). T cells activated in ImmunoCult XF media displayed a rapid increase in cellular reducing capacity, plateauing at 72 hours, while T cells activated in TexMACS media exhibited a slower and continuous rise in reducing potential up to 72 hours (FIG. 7F).

Media composition not only impacts the kinetics of metabolic changes, but also influences the metabolic landscape of activated T cells overall. Uniform Manifold Approximation and Projection (UMAP) of OMI parameters showed clustering based on culture media rather than the type of activating antibodies used (FIG. 7G, H), indicating that metabolic features of activated T cells were predominantly determined by media composition. Distinct OMI metabolic profiles also allowed classification of T cells activated in ImmunoCult XF media and TexMACS media with high sensitivity and specificity (AUC>0.93) across several classifier models (FIG. 71). Overall, the data demonstrate that while increased glycolysis upon activation is robust across several activation conditions, media composition controls the metabolic kinetics and ATP production of activated T cells, with faster changes induced by glucose^{high}/glutamine^{high} ImmunoCult XF media, regardless of the activating antibody used.

### OMI Indicates Cell Cycle Progression in Activated T Cells

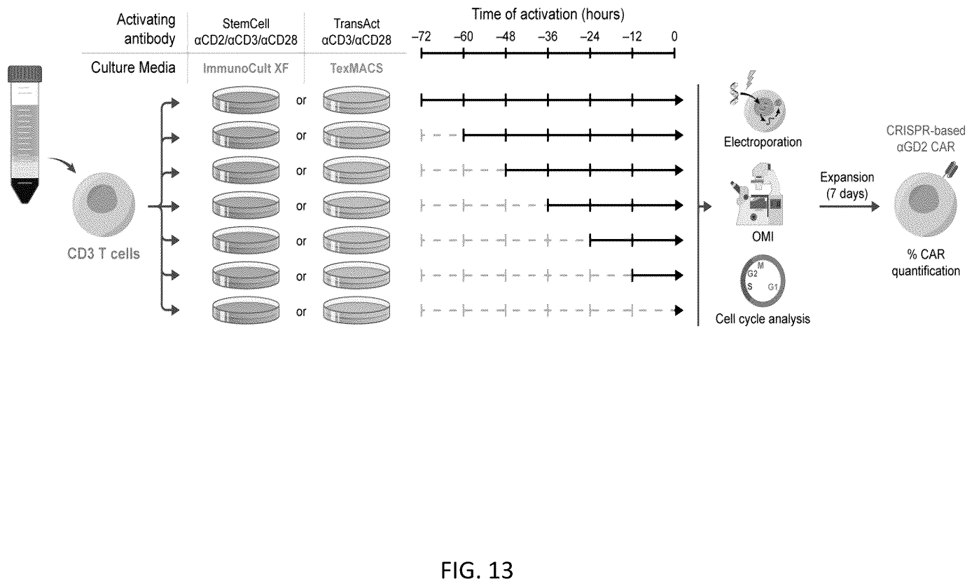

Since data reveal the impact of media composition on T cell metabolism and energy production, both of which are directly involved in proliferation, it was investigated how different activation conditions affect cell cycle progression of activated T cells. Using OMI, T cell metabolism was characterized every 12 hours following two activation conditions: StemCell αCD2/αCD3/αCD28 antibody in ImmunoCult XF media (Imm) or TransAct αCD3/αCD28 antibody in TexMACS media (Tex) (FIG. 8A, FIG. 13). The signature decrease in NAD(P)H τₘ and increase NAD(P)H α₁ were detected in both Imm- and Tex-activated T cells as early as 12 hours post activation (FIG. 8B-D). However, distinct metabolic kinetics was observed among these cells. Consistent with the findings above, Imm-activated T cells reached peak metabolic changes (lowest NAD(P)H τₘ and highest NAD(P)H α₁) faster than Tex-activated T cells (36-48 hours compared to 48-72 hours post-activation, respectively) (FIG. 8C, D). Consistently greater effect sizes (Glass's delta) of the Imm activation condition on NAD(P)H τₘ were observed and NAD(P)H α₁ compared to the Tex activation condition across three donors (Table 3). These findings further support that glucose^{high}/glutamine^{high} ImmunoCult XF media induced not only faster but also more pronounced metabolic changes in activated T cells compared to glucose^{low}/glutamine^{low} TexMACS media. Besides metabolic changes, T cells also underwent a significant increase in cytoplasm size following activation by Imm and Tex methods, indicating that they are primed for proliferation (FIG. 8E).

TABLE 3
Glass's deltas (Δ) showing maximal effect sizes of Imm and
Tex activation methods on T cell metabolism (NAD(P)H τₘ and NAD(P)H
α₁). For each donor and activation method, Glass's deltas were calculated
based on NAD(P)H τₘ and NAD(P)H α₁ at the time point (12,
24, 36, 48, 60, or 72 hours of activation duration) of maximal
differences compared to donor-matched quiescent T cells (0-hr
activation duration) in respective media (Imm or Tex).

| Variable | |
|---|---|
| ΔNAD(P)H τₘ | ΔNAD(P)H α₁ |

Activation method

| | Imm | Tex | Imm | Tex |
|---|---|---|---|---|
| Donor 1 | 2.61 | 1.56 | 3.76 | 2.50 |
| Donor 2 | 3.55 | 1.66 | 5.53 | 3.35 |
| Donor 3 | 2.18 | 1.57 | 4.64 | 2.47 |

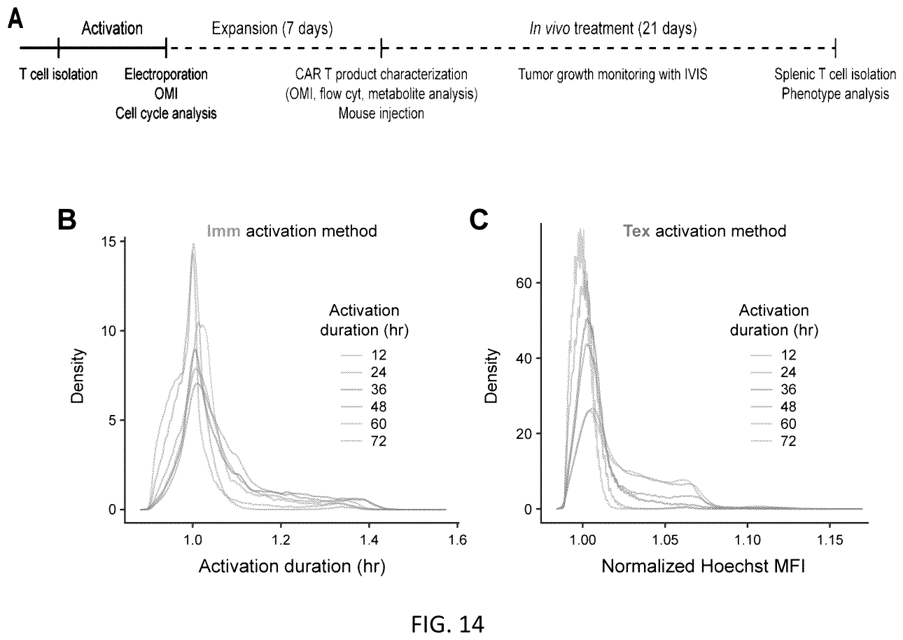

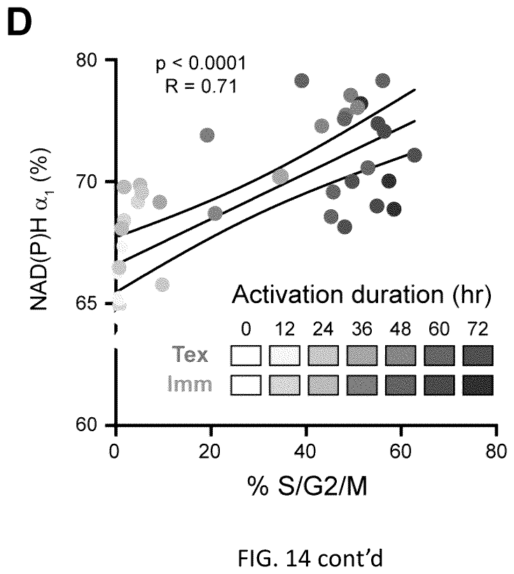

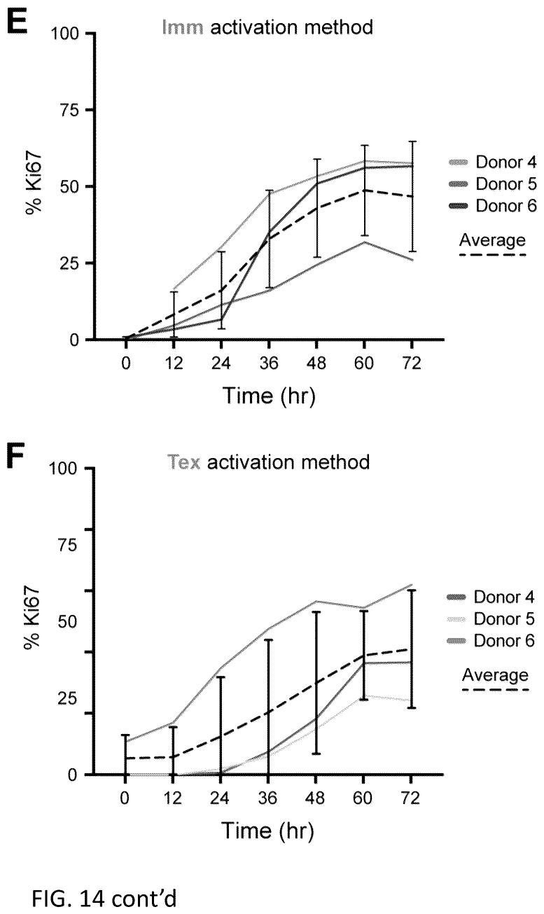

Following activation with either Imm or Tex methods, T cells rapidly progressed through the cell cycle with increased DNA content as measured via flow cytometry of Hoechst stain (FIG. 14B, C). Consistent with the metabolic kinetics observed with OMI, Imm-activated T cells displayed an earlier increase in the percent of cells in S/G2/M phase (% S/G2/M) (36-48 hours) compared to Tex-activated T cells (60-72 hours) (FIG. 8F, G, FIG. 14E, F where error bars are removed for clarity). Correlation analysis confirmed the relationship among OMI metabolic features and cell cycle progression. Across three donors and two activation methods, significant (p<0.0001) correlations between OMI measurements and % S/G2/M were found (FIG. 8H, I, FIG. 14D). Specifically, % S/G2/M correlated negatively with NAD(P)H τₘ and positively with NAD(P)H α₁ and cytoplasm size, suggesting that actively cycling T cells had low NAD(P)H τₘ, more free NAD(P)H, and large cytoplasm size. T cell proliferation (% Ki-67⁺ cells) also trended upwards throughout the 72-hour activation time course (FIG. 14E, F). In summary, using non-invasive, label-free OMI measurements, it has been demonstrated the influence of media composition on cell cycle progression following activation, and the faster metabolic changes induced by glucose^{high}/glutamine^{high} ImmunoCult XF media support earlier cell cycle entry compared to glucose^{low}/glutamine^{low} TexMACS.

### OMI Identifies Features of T Cells that Predict Optimal CAR Gene Transfer Conditions

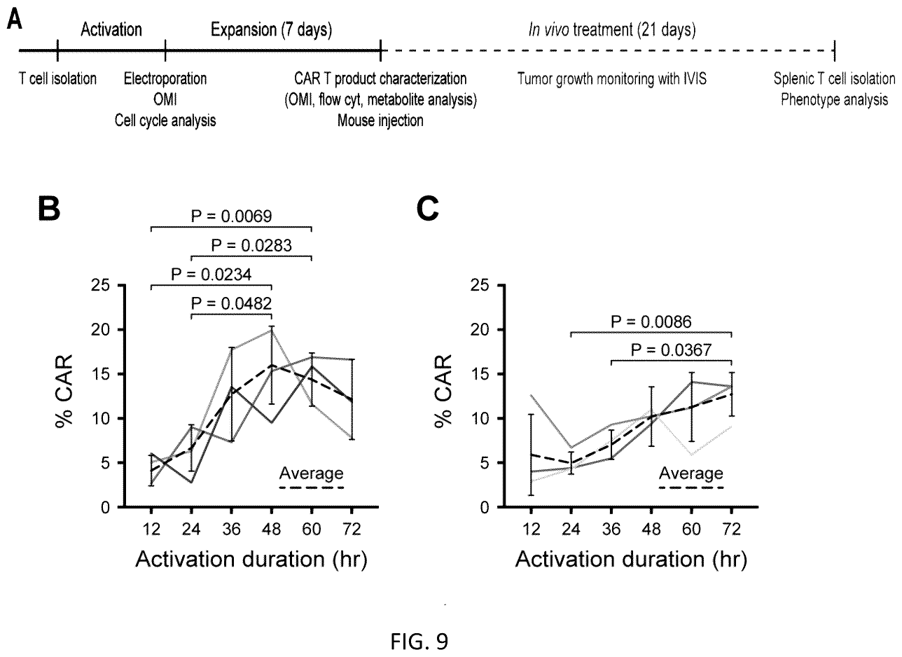

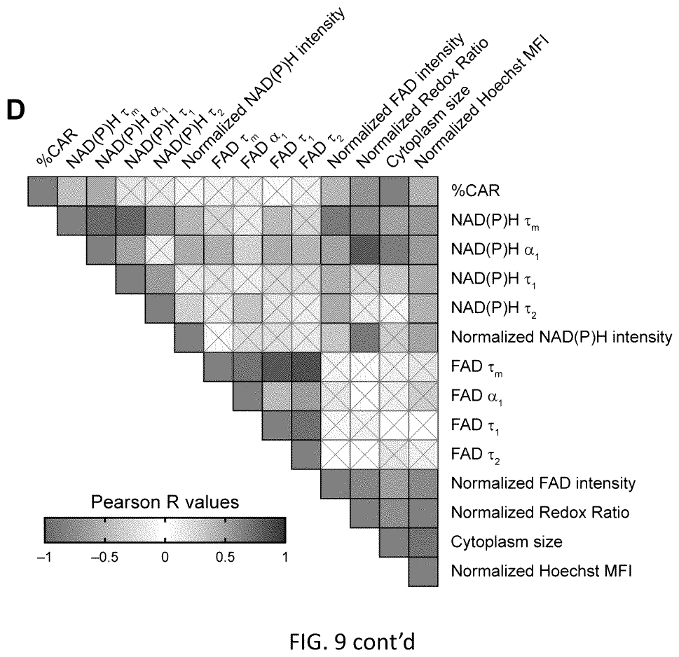

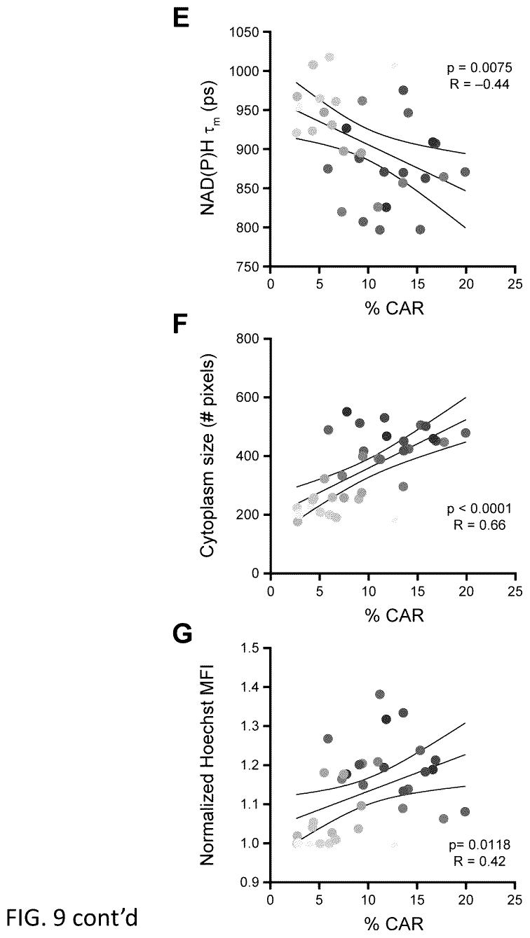

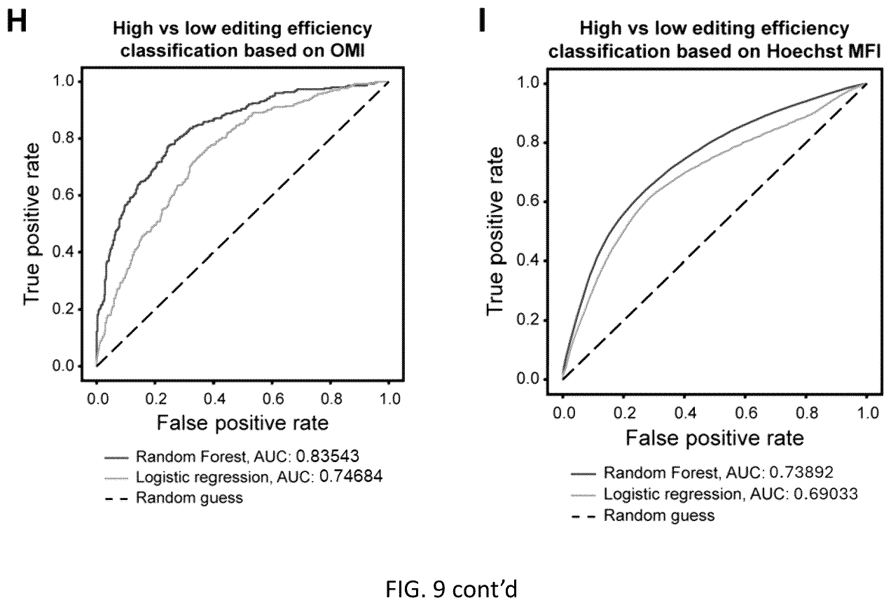

Activation primes T cells for CAR gene transfer; however, the data indicate that different activation conditions yield different T cell metabolic profiles and cell cycle progression kinetics. Thus, it was further investigated how OMI characteristics of T cells at the gene transfer timepoint affected transgene incorporation efficiency. Anti-GD2 CAR transgene was introduced using either retroviral transduction (FIG. 15) or electroporation-based CRISPR/Cas9 (FIG. 9). Briefly, T cells isolated from two healthy donors were activated (day 0) and underwent T-cell receptor alpha-constant knockout (TRAC KO) (day 2) prior to retroviral transduction with two anti-GD2 CAR constructs (14G2a-OX40-CD28-ζ CAR or 14G2a-41BB-ζ CAR) (day 4) as previously described (FIG. 15A) (9). At the transduction timepoint (day 4), TRAC KO T cells displayed significantly lower NAD(P)H τₘ, higher NAD(P)H α₁, and greater cytoplasm size-OMI features of cycling cells—compared to control T cells with intact T cell receptor (FIG. 15B-E). Significantly higher transduction efficiency was observed, quantified as percent CAR positivity post-expansion (day 11), in TRAC KO cells compared to control cells (FIG. 15F). This suggests that metabolic and morphological characteristics measured by OMI at the time of transduction can indicate the condition that increases transgene incorporation, and, hence, CAR yield.

Besides viral vectors, CRISPR/Cas9 is currently being explored for future CAR T therapies as it allows precise genome editing that improves treatment safety and efficacy (39, 40). Therefore, it was further characterized whether OMI features could inform CRISPR genome editing efficiency. Following 12-to-72 hour activation by either Imm or Tex methods, T cells were imaged with OMI and electroporated to generate CRISPR-edited anti-GD2 CAR T cells as previously described (41). Notably, across three donors, the timeframe that yielded optimal genome editing efficiency, quantified as percent CAR positivity post expansion (FIG. 9B, C), aligned with the timeframe of maximal decrease in NAD(P)H τₘ and increase in NAD(P)H α₁ (FIG. 8C, D). These OMI characteristics (low NAD(P)H τₘ and high NAD(P)H α₁) were consistent with those displayed by T cells that yielded high transduction efficiency (FIG. 15). For Imm-activated T cells, electroporation at 36-60 hours post-activation yielded the highest CAR gene transfer efficiency (FIG. 15B). Meanwhile, Tex-activated T cells showed increased % CAR positivity as the activation duration at electroporation increased, with the highest genome editing efficiency observed for electroporation at 48-72 hours post-activation (FIG. 15C). Electroporation at the optimal timeframe yielded a 3- to 4-fold-increase in genome editing efficiency.

Overall, the data reveal a robust relationship between OMI metabolic features, specifically low NAD(P)H τₘ and high NAD(P)H α₁, and CAR transgene incorporation across both retroviral transduction and electroporation-based CRISPR/Cas9.

### OMI Features of T Cells are More Sensitive and Specific than Cell Cycle Analysis by Flow Cytometry to Predict Genome Editing Efficiency

Unlike label-free non-invasive OMI, cell cycle analysis based on S/G2/M and Ki-67 measurements from flow cytometry are destructive to T cells and involve exogenous labelling of the culture. As distinct OMI features in T cell groups with high versus low transgene incorporation efficiency were observed, it was also investigated whether label-free OMI measurements could inform the electroporation timepoint decision and how its sensitivity and specificity compared to that of flow cytometry measurements. Correlation analysis across 36 samples from 3 donors revealed significant (p<0.05) and moderate (|R|>0.4) correlations, indicating a robust relationship, between genome editing efficiency (percent CAR positivity) post expansion and several OMI measurements at electroporation (FIG. 9D-F, FIG. 15B, C). Hoechst median fluorescence intensity (MFI) of activated T cells at electroporation also significantly correlated with genome editing efficiency, though the R-value was slightly lower than those of OMI variables [R=0.42 (FIG. 9G) compared to R=−0.44 and R=0.66 (FIG. 9E-F)]. Gating based on Hoechst MFI yielded the percentage of cycling cells (% S/G2/M), which also correlated with % CAR positivity 7 days later (FIG. 15D). Similarly, proliferative capacity (% Ki-67⁺) in T cells also showed significant (p<0.0001) and strong positive correlations with genome editing outcome (FIG. 15E). These findings align with prior research showing that genome editing during mitosis increases transgene incorporation efficiency. Additionally, the data indicate the potential of OMI to identify gene transfer conditions that maximize CAR T cell yield.

To further assess the accuracy of OMI for guiding the electroporation timepoint decision, machine learning was used to predict cells from samples that later exhibit high versus low editing efficiency (high efficiency is considered as >15% CAR⁺ for Imm-activated T cells and >10% CAR⁺ for Tex-activated T cells). A Random Forest classifier achieved good sensitivity and specificity (AUC=0.83) in predicting genome editing efficiency (day 7) based on OMI parameters at the time of electroporation (day 0) (FIG. 9H). Meanwhile, models trained on Hoechst MFI to predict optimal electroporation conditions did not perform as well (AUC=0.74) despite 150× more cells used for training (FIG. 9I). Overall, T cell metabolism and morphology, as quantified by non-invasive label-free OMI, can inform when activated T cells are optimal for CAR gene transfer to increase CAR yield.

### OMI and Phenotypic Markers Separate CAR T Cells by Media Composition

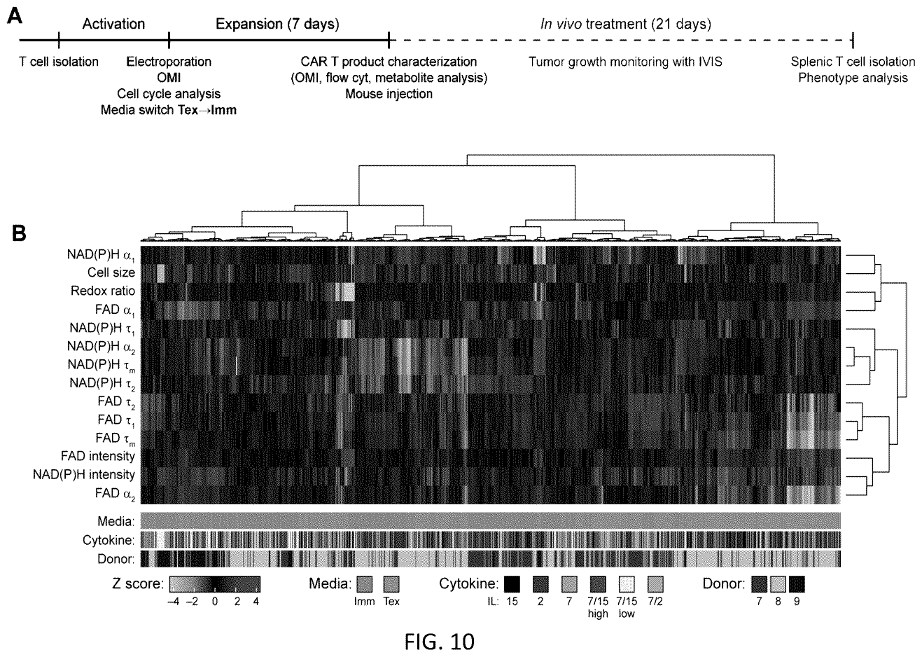

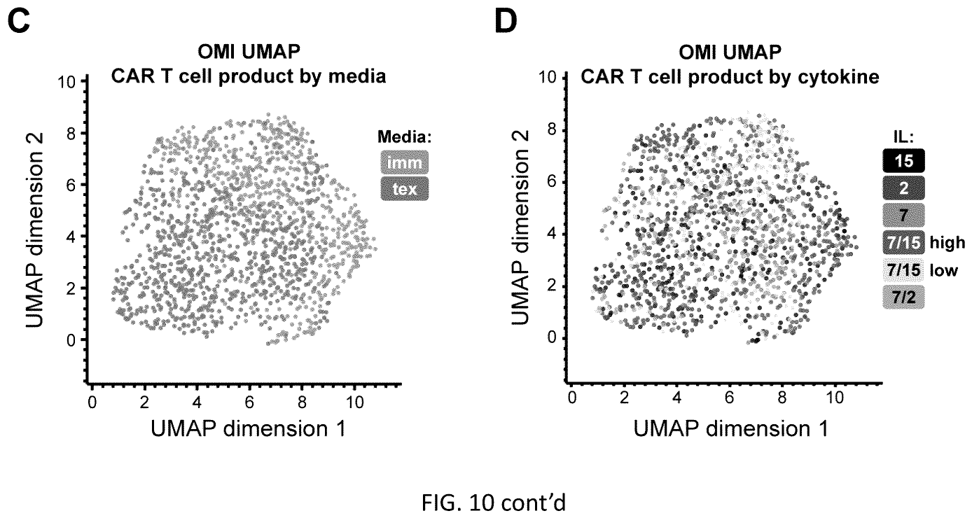

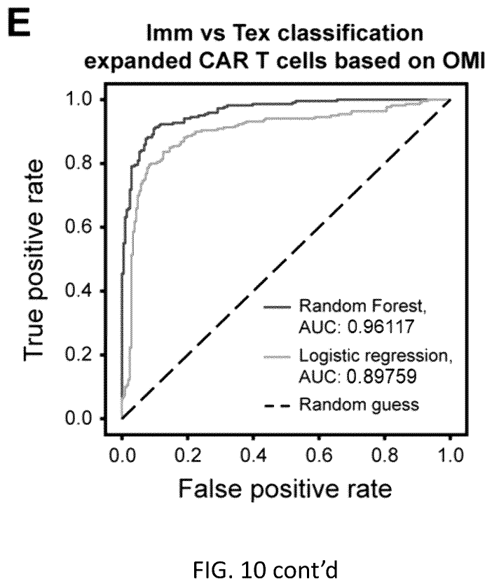

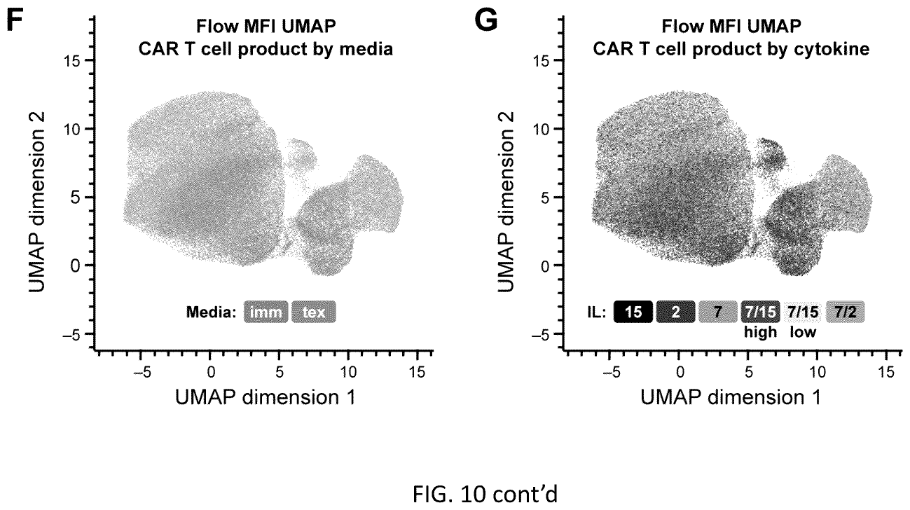

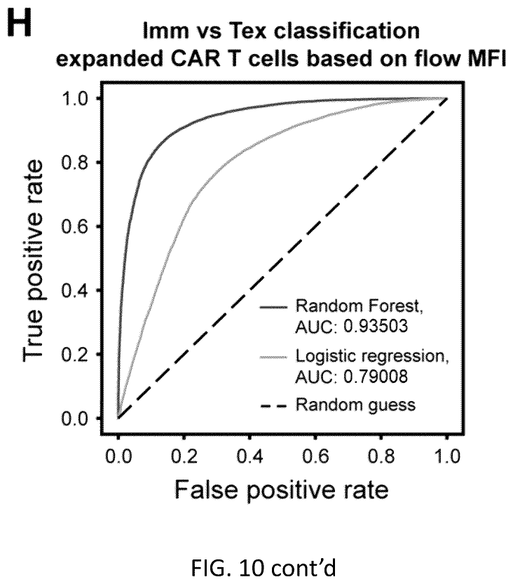

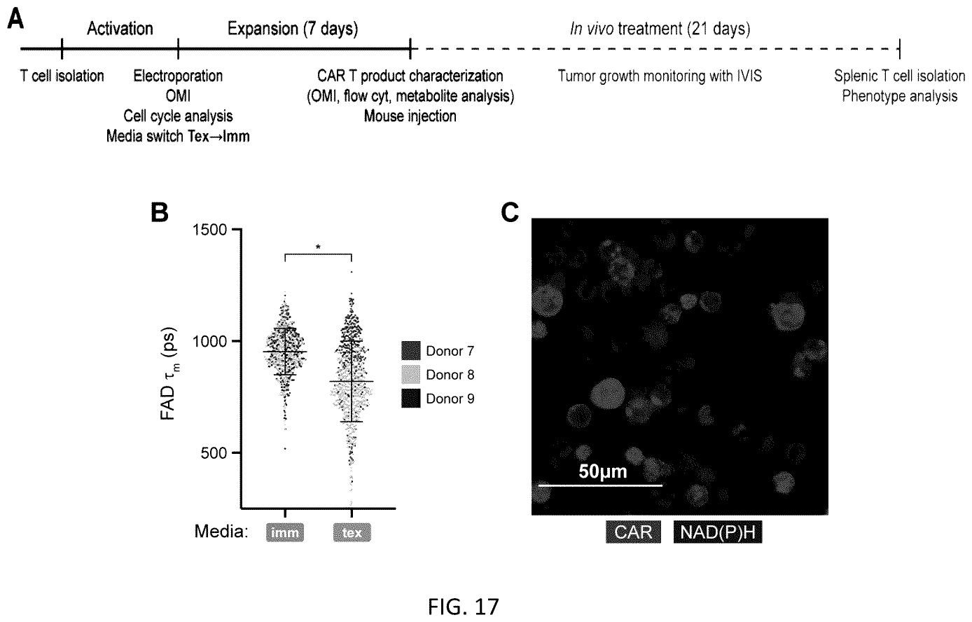

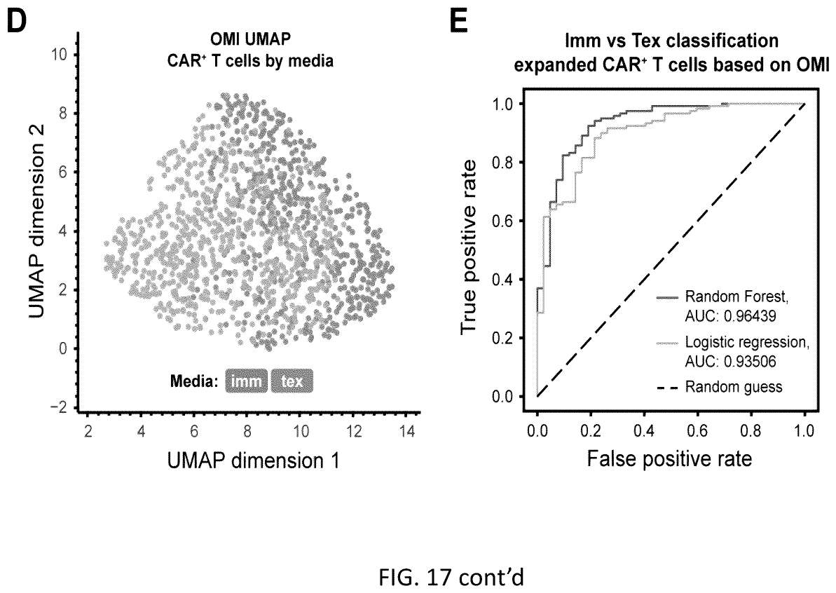

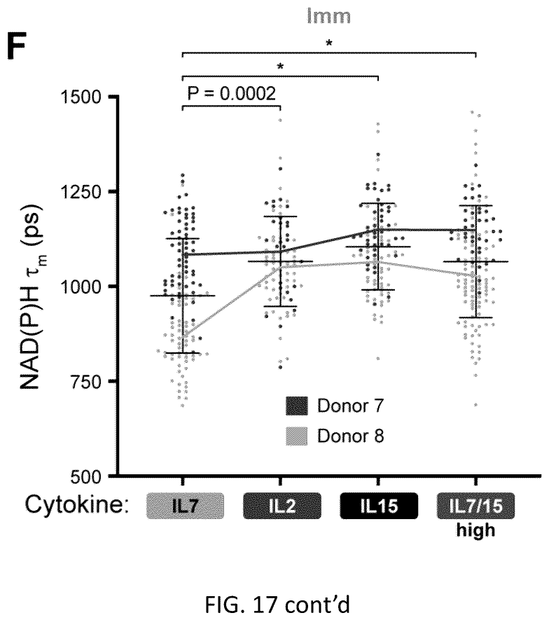

Ex vivo expansion following CAR gene transfer not only achieves sufficient CAR T dosage, but also shapes the metabolic and phenotypic features of the resultant CAR T products, which highly correlate to potency and treatment outcomes. As OMI measurements have identified the role of media composition on T cell metabolism during the activation and gene transfer phases of CAR T manufacturing, it was further evaluated how two media compositions (ImmunoCult XF and TexMACS, as above) in combination with various cytokines (IL-2, IL-7, IL-7/IL-2, and IL-7/IL-15) control features of CRISPR-edited anti-GD2 CAR T cells. OMI and flow cytometry of 6 naïve/stem memory markers were used to evaluate CAR T cell metabolism and phenotypes under several expansion conditions. Both heatmap and UMAP of OMI measurements from CAR T cell products post-expansion (which included both CAR⁺ and CAR⁻ cells) revealed distinct clustering based on culture media rather than supplemented cytokines (FIG. 10B-D). Notably, CAR T cells expanded in TexMACS media exhibited lower FAD mean lifetime (FAD τₘ), regardless of cytokines used (FIG. 17B). Machine learning algorithms achieved high sensitivity and specificity in classifying CAR T cells expanded in ImmunoCult XF media and TexMACS media based on their OMI features, further confirming their distinct metabolic profiles (AUC>0.96) (FIG. 10E). Similar trends were observed in the CAR⁺ fraction with clustering based on culture media in a UMAP of OMI parameters (FIG. 17C, D) and high accuracy for classification of CAR⁺ cells by media condition based on OMI features (FIG. 17E). Consistently higher NAD(P)H τₘ was observed in CAR T cells expanded in IL-7±IL-15 compared to those expanded in IL-2 in ImmunoCult XF media (FIG. 17F), suggesting that CAR T cells did modulate their metabolism based on the cytokines present, though to a lesser extent than the culture media.

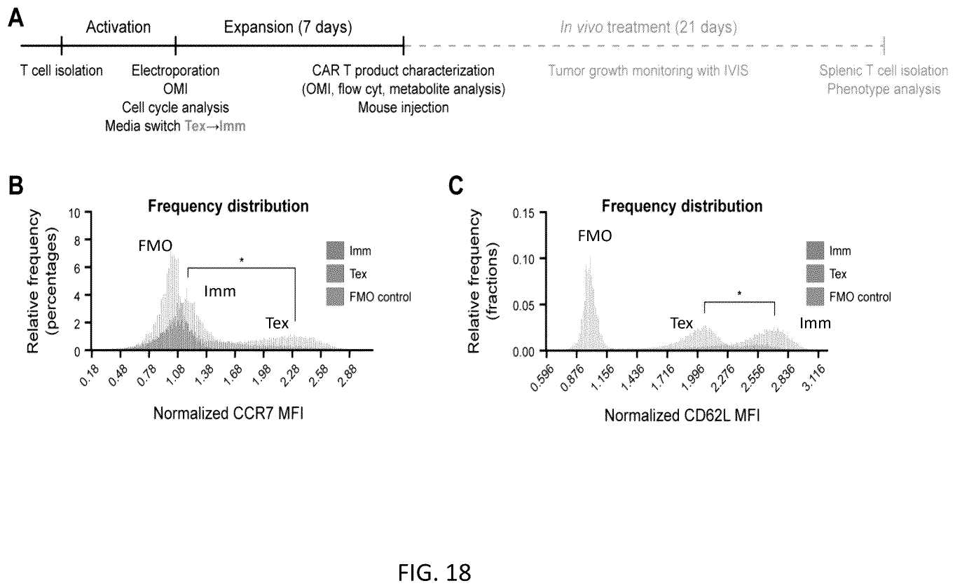

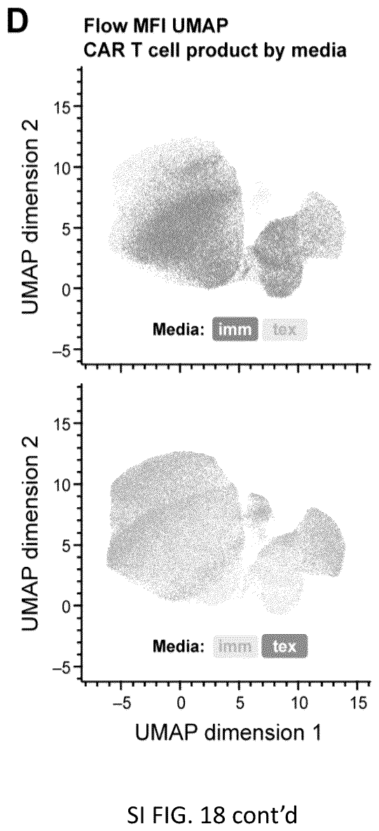

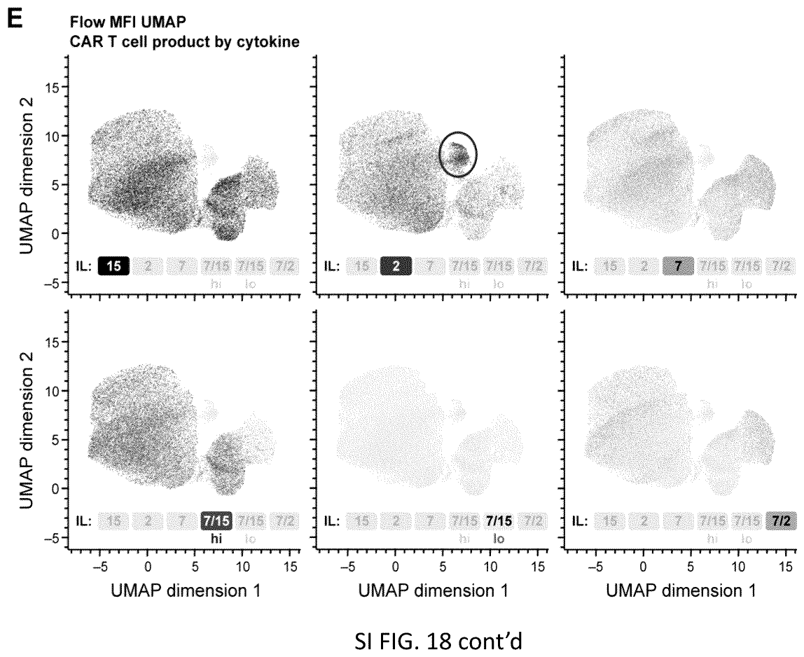

Media composition also had dominating impacts on CAR T cell phenotype (FIG. 10F-H). Flow cytometric analysis revealed significantly higher CCR7 expression and lower CD62L expression in CAR T cells expanded in TexMACS compared to those expanded in ImmunoCult XF media (FIG. 18B, C). UMAP based on surface marker expression also showed distinct clusters of CAR T cells expanded in ImmunoCult XF versus TexMACS media, regardless of cytokines used (FIG. 10F, G). Consistently, machine learning algorithms trained on surface marker expression to classify CAR T cells expanded in ImmunoCult XF versus TexMACS media also achieved high sensitivity and specificity (AUC=0.97) (FIG. 10H). Interestingly, CAR T cells expanded in IL-2 showed a distinct phenotypic island that was not observed in other cytokine conditions (circle, FIG. 18E). However, the impact of cytokines on CAR T cell phenotype was still secondary compared to culture media composition, shown by overlapping clusters of cytokine conditions in the OMI and surface marker UMAPs (FIG. 10D, G).

### OMI Reveals Distinct Metabolic Profile of CAR T Cells Undergoing Media Switch from TexMACS to ImmunoCult XF

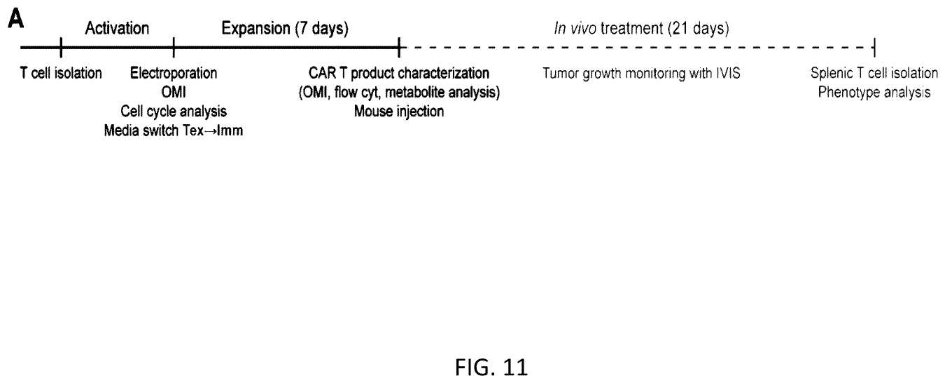

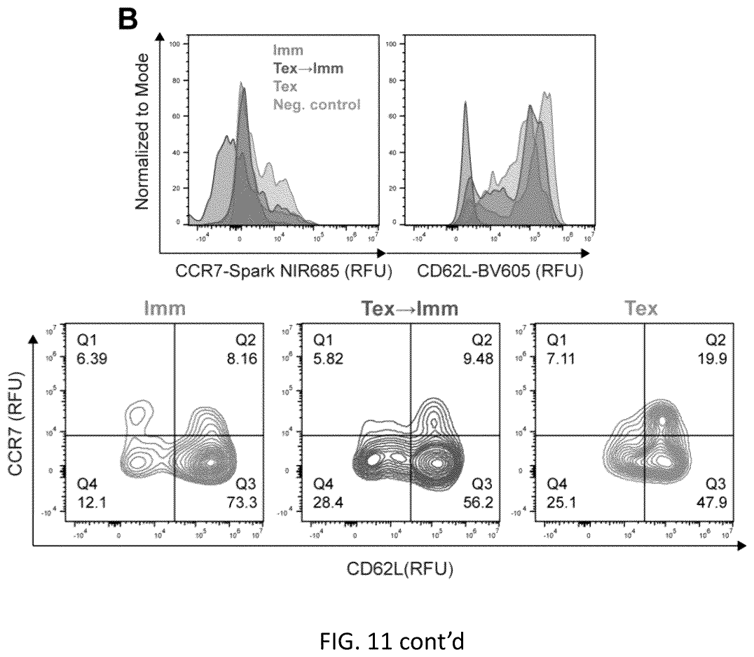

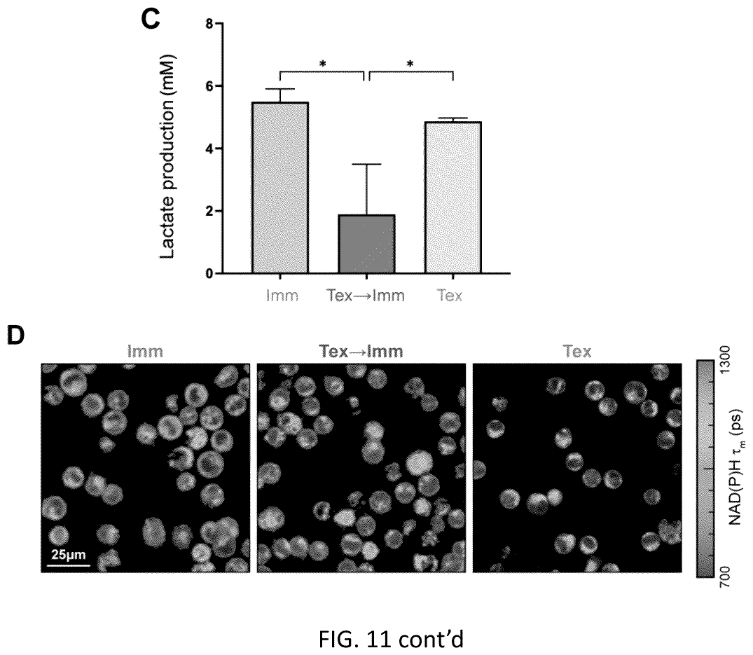

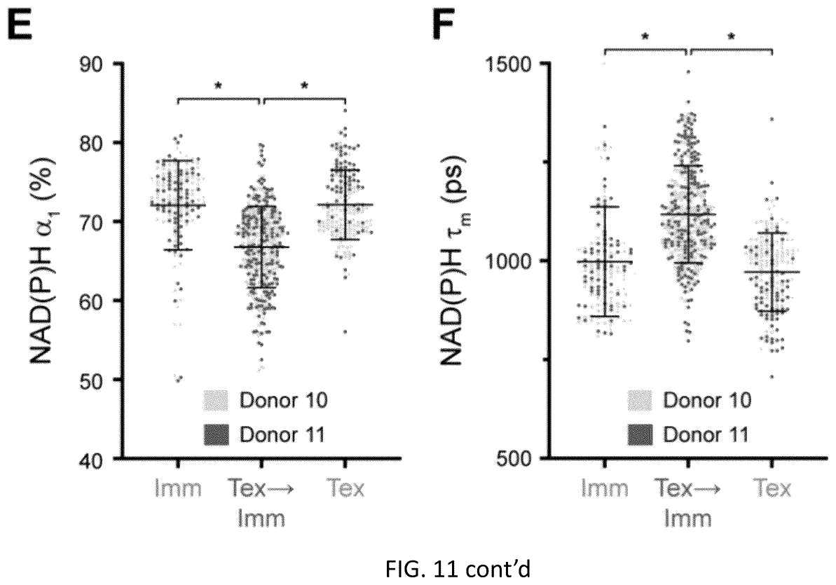

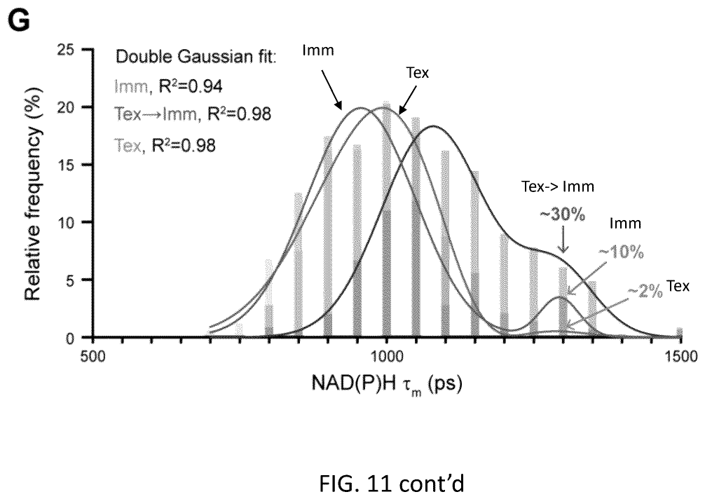

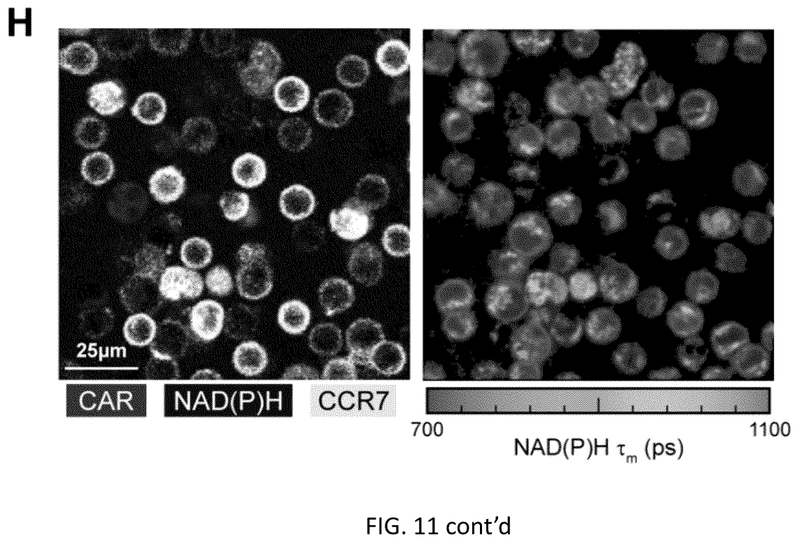

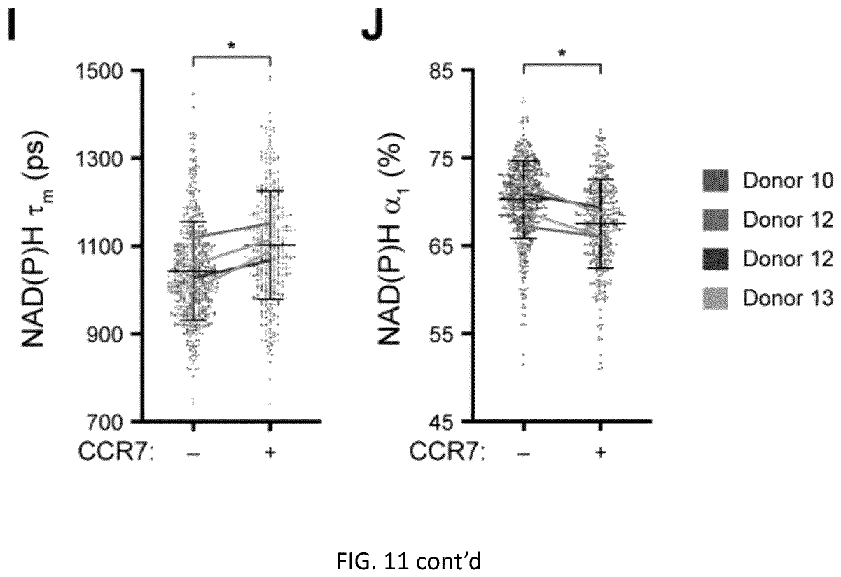

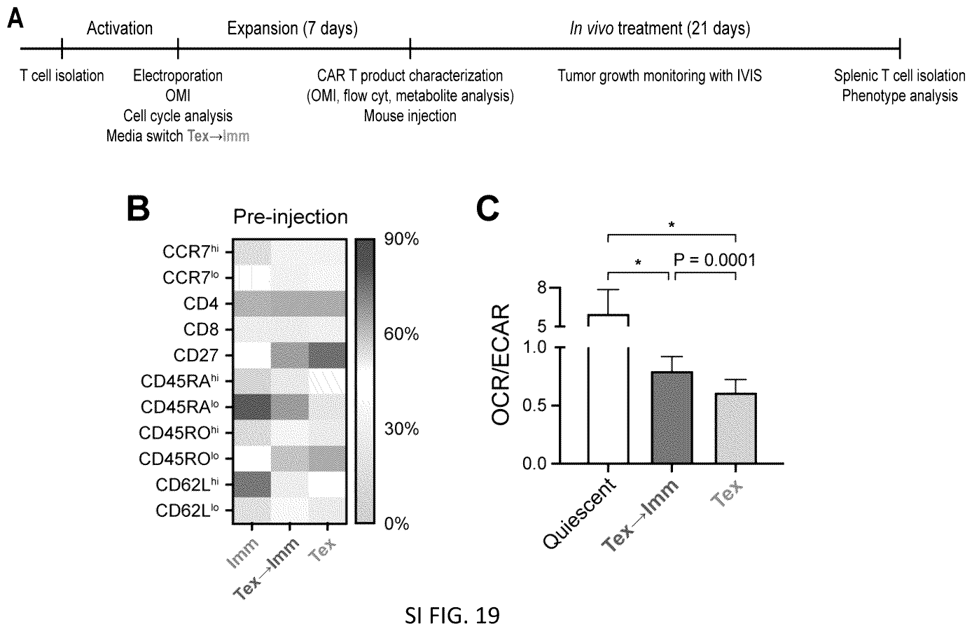

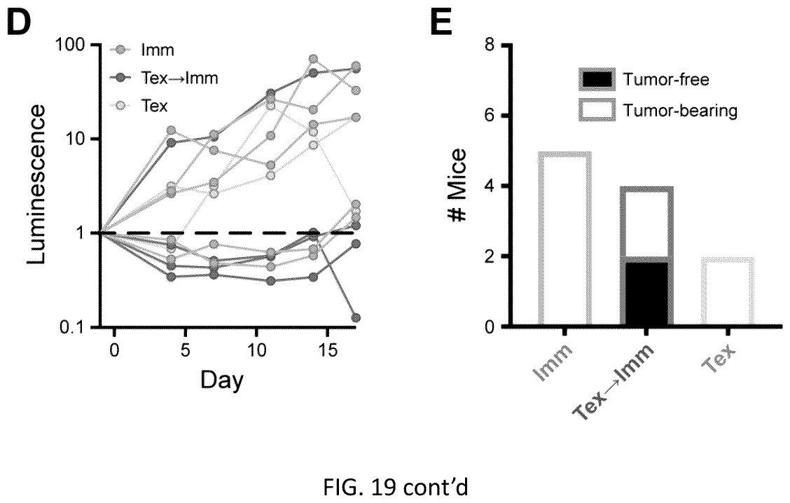

Transient glucose or glutamine restriction during manufacturing has been shown to produce CAR T cells with higher metabolic fitness, characterized by greater mitochondrial mass and more oxidative activity, that correlates to better in vivo potency and persistence (18, 42, 43). As CAR T cells expanded in ImmunoCult XF versus TexMACS media exhibited different phenotypic and OMI profiles (FIG. 10), it was further investigated how CAR T cell metabolic profile and function are impacted by changing the expansion condition from TexMACS+10 ng/ml IL-7 pre-electroporation, which served as a transient glucose and glutamine restriction, to ImmunoCult XF+500U/mL IL-2 post-electroporation (FIG. 11, FIG. 19). Hereafter this condition is referred to as Tex→Imm.

Post-expansion and pre-infusion into a mouse xenograft model (FIG. 7a), distinct T cell phenotypes in CAR T cells expanded in the Tex→Imm condition were not observed compared to those expanded ImmunoCult XF (Imm) and TexMACS (Tex) media alone. CAR T cells expanded in Tex→Imm displayed an intermediate phenotype based on naïve and stem memory markers CCR& and CD62L (FIG. 11B, Supplementary FIG. 19B). However, significantly less lactate secreted by Tex→Imm CAR T cells was observed, suggesting lower glycolytic activity, compared to CAR T cells cultured in a singular media composition (FIG. 11C). This observation was further supported by metabolic flux analysis that showed a significantly higher ratio of oxygen consumption rate (OCR) to extracellular acidification rate (ECAR) in non-edited T cells expanded in the Tex→Imm condition compared to those cultured in Tex media alone. The high OCR/ECAR ratio indicated an increased dependence on oxidative metabolic pathways in Tex→Imm group (FIG. 19C).

OMI also captured a distinct metabolic profile in CAR T cells expanded in Tex→Imm, which had significantly lower NAD(P)H α₁ and higher NAD(P)H τₘ than CAR T cells cultured in Imm or Tex media alone (FIG. 11E, F). As OMI allows single-cell measurements, it was further investigated the population heterogeneity in CAR T cells expanded within the Imm, Tex, and Tex→Imm conditions. Two subpopulations were observed in each CAR T cell group post-expansion, displaying high- and low-NAD(P)H τₘ respectively (FIG. 11G). Notably, the high NAD(P)H τₘ subpopulation made up a greater proportion in Tex→Imm CAR T cells (~30-40%) compared to the other two media conditions (10% in Imm and <5% in Tex CAR T cells). Additionally, independent OMI analysis of CCR7⁺ T cells from four donors showed significantly higher NAD(P)H τₘ and lower NAD(P)H α₁ than CCR7⁻ cells (FIG. 11H-J). This suggests a potential shift towards stem-like metabolism in Tex→Imm CAR T cells, which had more cells in the high-NAD(P)H τₘ subset (FIG. 11G, I). Overall, the distinct OMI metabolic profile of Tex→Imm CAR T cells correlates to a lower glycolytic rate and higher dependence on oxidative pathways that can potentially improve in vivo persistence and potency (20, 42).

Additionally, independent OMI analysis of CCR7⁺ T cells from four donors showed significantly higher NAD(P)H τₘ and lower NAD(P)H α₁ than CCR7⁻ cells (FIG. 11H-J). This suggests a potential shift towards stem-like metabolism in Tex→Imm CAR T cells, which had more cells in the high-NAD(P)H τₘ subset (FIG. 11G, I). Overall, the distinct OMI metabolic profile of Tex→Imm CAR T cells correlates to a lower glycolytic rate and higher dependence on oxidative pathways that can potentially improve in vivo persistence and potency (20, 42).

### CAR T Cells Undergoing Media Switch from TexMACS to ImmunoCult XF Yielded Better In Vivo Potency

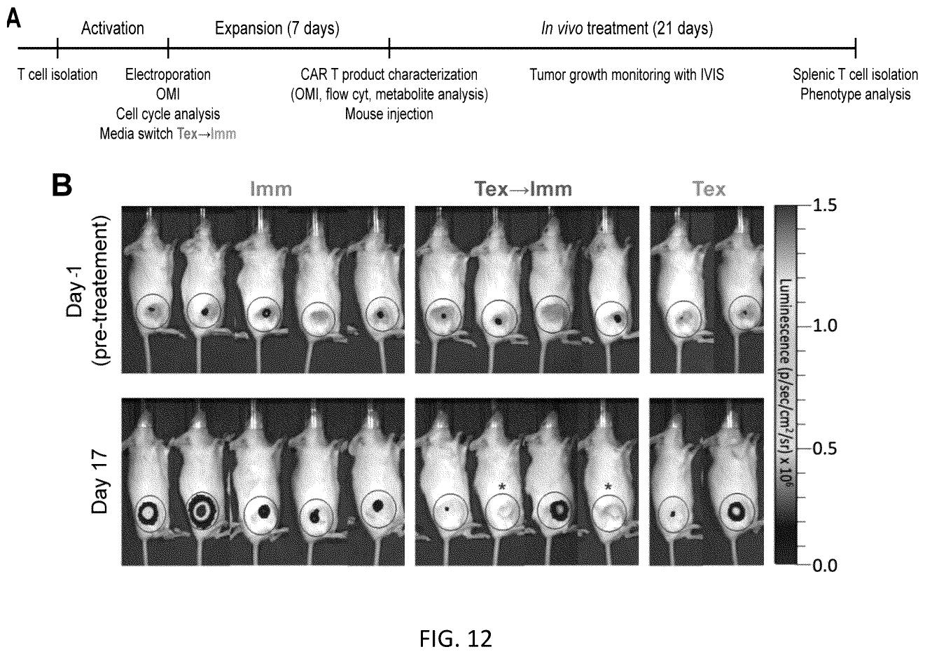

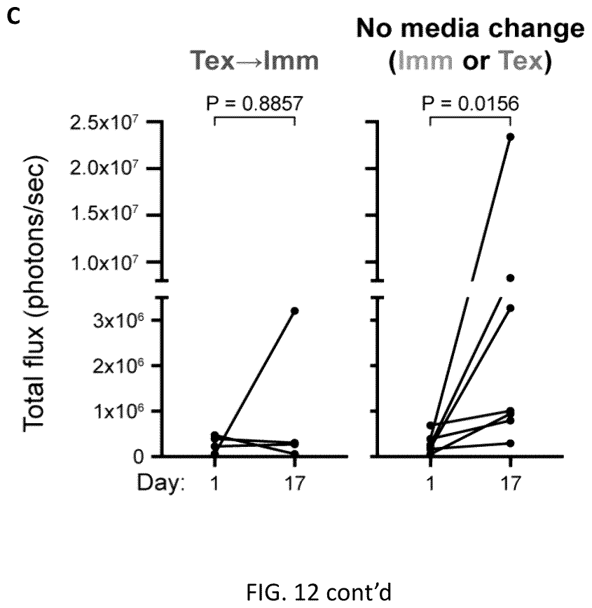

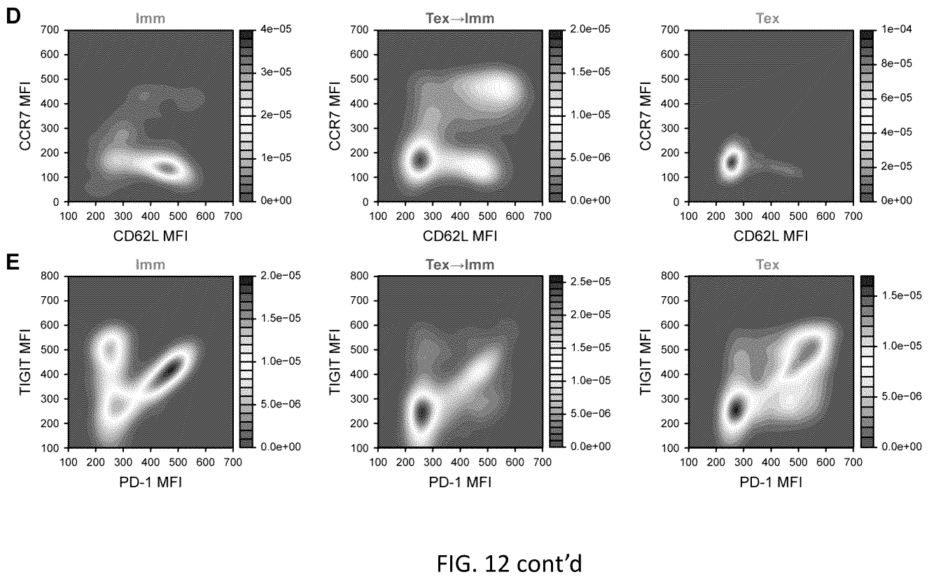

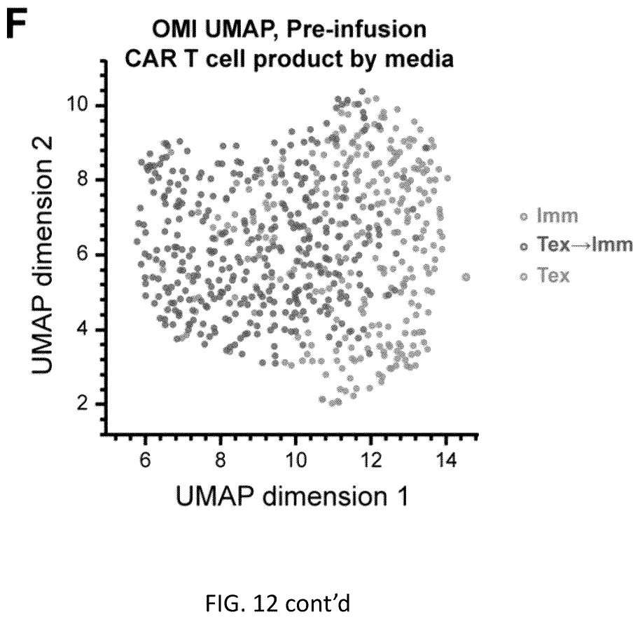

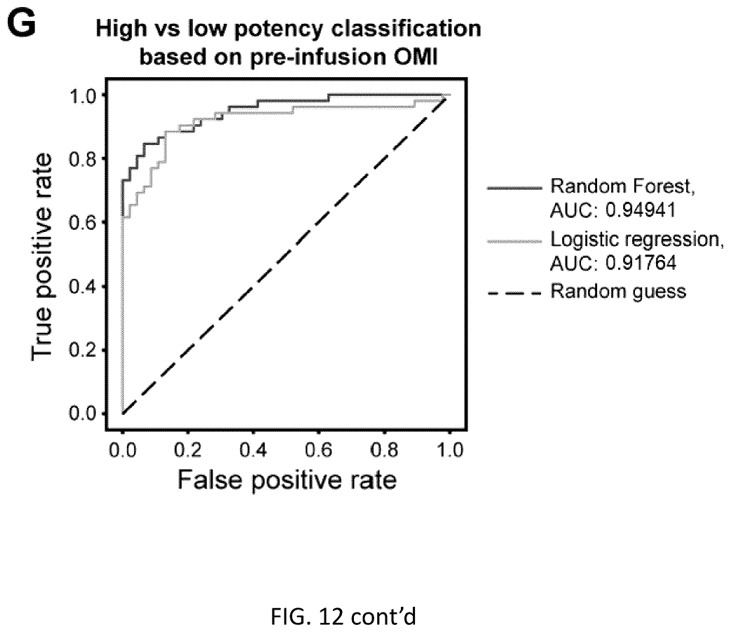

As OMI and other metabolic assays revealed a distinct metabolic profile expressed by Tex→Imm CAR T cells, it was further assessed how different expansion conditions influenced in vivo potency of CRISPR-edited anti-GD2 CAR T cells. NSG mice with established GD2⁺ CHLA-20-Luciferase neuroblastoma xenografts were treated with 4 million CAR⁺ T cells expanded under Imm, Tex, or Tex→Imm media conditions (FIG. 12A). By day 17 post treatment, CHLA-20 bioluminescence increased significantly in cohorts treated with CAR T cells expanded in Imm or Tex media alone, indicating tumor progression (FIG. 12B, C). Notably, CAR T cells expanded in Tex→Imm condition led to tumor regression (fold change in tumor flux <1) in two out of four mice, and better control of tumor flux overall (FIG. 12B, C, FIG. 19D, E). Interestingly, CD45+ human T cells isolated from the mouse spleen 21 days post treatment with Tex→Imm CAR T cells showed better retention of stem cell memory phenotype (CCR7⁺/CD62L⁺) and resistance to exhaustion in vivo (PD-1⁺/TIGIT⁺) compared to CAR T cells expanded by singular media (FIG. 12D, E) (43, 44). This suggests that the distinct OMI metabolic profile observed in Tex→Imm CAR T cells ex vivo confers functional advantages in vivo.

The distinct metabolic profiles expressed by Tex→Imm CAR T cells pre-infusion are hypothesized to underlie their persistence and efficacy in vivo. To test this, it was investigated whether pre-infusion OMI metabolic features could be used to predict CAR T in vivo potency. A UMAP of OMI parameters (Table 2) from pre-infusion CAR T products revealed different clusters of Tex→Imm expanded CAR T cells versus CAR T cells expanded in singular media (FIG. 12F). Machine learning classification based on pre-infusion OMI metabolic profiles successfully distinguished high potency (Tex→Imm) from low potency (Imm or Tex alone) CAR T cells with high sensitivity and specificity (FIG. 12G; AUC=0.90). These findings align with previous research indicating better in vivo potency by stem cell memory T cells and suggest that OMI measurements of CAR T cells ex vivo can predict in vivo response (FIG. 12G).

### Discussion

The translation of CAR T cell therapies to solid tumors is challenging, partially due to a complex manufacturing process that requires optimization at several stages. Current methods to assess T cell function are invasive, involving handling and sampling an active cell culture, and mainly rely on final product parameters to satisfy release criteria. Thus, they do not capture the dynamic and heterogenous changes in CAR T cells throughout manufacturing, including metabolic changes. This limits the ability to fine-tune manufacturing conditions to achieve the optimal CAR T cell profile. Recent research suggests that T cell metabolism correlates with function, and metabolic reprogramming ex vivo can achieve desirable functions in vivo (20, 22, 42, 43). Here, it has been demonstrated that OMI, a label-free optical imaging technique, can non-invasively monitor T cell metabolism during CAR T cell manufacturing and OMI features can be used to determine the optimal manufacturing conditions to improve CAR T cell products.

The results highlight that nutrient availability and media composition modulates the strength and speed of T cell activation, while also controlling cell cycle entry and differentiation fate. Two common T cell culture media were examined-ImmunoCult XF and TexMACS-which have different concentrations of key nutrients that impact T cell metabolism and functional fates. Specifically, ImmunoCult XF media has higher glucose and glutamine concentrations, while TexMACS media contains GlutaMax, which can be hydrolyzed into L-glutamine and L-alanine by cell-secreted aminopeptidases (46). Interestingly, activation in ImmunoCult media induced faster and greater changes in T cell metabolism, ATP production, and cellular reducing potential. Similarly, expansion in ImmunoCult XF and TexMACS media also yielded CAR T cells with distinct metabolic and phenotypic profiles. In the study, the effects of media composition on T cell metabolism and function dominated over the effects of different activating antibodies and supplemented cytokines. These observations emphasize the importance of optimizing media formulations in the clinical development of CAR T cells.

Metabolic shifts during mitosis have been documented, with high aerobic glycolysis and glutaminolysis for energy production and biomass synthesis. The data reveal a correlation between T cell OMI features, notably high NAD(P)H α₁ and low NAD(P)H τₘ, and cell cycle stage analysis by flow cytometry of Hoechst and Ki-67 stains. Previously, high NAD(P)H α₁ and low NAD(P)H τₘ were associated with cell cycle entry in Kasumi-1 cells (human acute myeloid leukemia progenitors). This suggests that OMI may be sensitive to metabolic changes due to the cell cycle across several human cells, and that label-free OMI measurements can be a good indicator of cell cycle progression in T cells following activation.

Successful incorporation of the CAR transgene is required for the expression of functional CAR receptors to mediate therapeutic efficacy. The efficiency of several gene transfer methods is controlled by cell cycle, as mitotic cells have high homology directed repair that enables precise, on-target genome editing. Several studies have attempted cell cycle synchronization to improve CAR transgene incorporation efficiency. Here, a robust relationship between OMI metabolic measurements and gene transfer efficiency across two platforms (viral transduction and electroporation-based CRISPR/Cas-9) has been demonstrated. Machine learning models based on label-free OMI measurements accurately identified gene transfer conditions that later resulted in high CAR transgene incorporation efficiency, indicating that OMI could inform the timing of genome editing to improve CAR yield.

Ex vivo metabolic perturbation during manufacturing, such as transient glucose or glutamine restriction, have been shown to reprogram CAR T cells epigenetically and prime them for the metabolically stressed, immunosuppressive in vivo tumor microenvironment (48, 49). It was found that CAR T cells undergoing media switch from glucose^{low} glutamine^{low} TexMACS pre-electroporation to glucose^{high} glutamine^{high} ImmunoCult XF post-electroporation exhibited distinct OMI profiles, with high oxidative phosphorylation and low glycolytic activity, together with improved in vivo potency. This is consistent with previous studies on the relationship between glycolysis and oxidative phosphorylation metabolism and T cell exhaustion and persistence (47-49), and suggests that modulating T cell metabolism based on different energy requirements at several manufacturing stages can improve CAR T efficacy. CAR T cell groups with high versus low in vivo potency were predicted based on pre-infusion OMI metabolic features with high sensitivity and specificity. This indicates that OMI could screen and inform manufacturing conditions that yield high potency, or predict treatment response based on ex vivo measurements pre-infusion, though further research is warranted. A substantial sub-population with higher NAD(P)H τₘ in CAR T cells expanded in Tex→Imm is identified that potentially contributes to their increased in vivo potency and persistence. These observations emphasize the significance of OMI's single-cell resolution in characterizing population heterogeneity to identify therapeutically beneficial subsets.

There are caveats to this study. T cells isolated from healthy donors were used as starting material for CAR T cell manufacturing, which does not fully capture characteristics observed in autologous T cells from cancer patients. Furthermore, the absence of endogenous TCR in the virus-free CRISPR TRAC KO anti-GD2 CAR T model may influence the T cells' response to media and cytokines. Further research should explore the effects of media composition and cytokines on CAR T cells with an intact TCR and validate the relationship between OMI measurements and in vivo potency across various CAR T cell and tumor models. Additionally, the current two-photon laser scanning OMI system can be adapted into other configurations such as a single-photon microscope, microfluidic devices (51), or flow cytometer (52, 53) to achieve faster speed, automation, and a smaller footprint to better integrate into CAR T manufacturing workflows. These technical efforts will expand the throughput and scope of OMI as a non-invasive, sensitive tool to support the translation of CAR T cell therapy for solid tumors.

In summary, the sensitivity and specificity of OMI for measuring T cell metabolism at several stages throughout CAR T manufacturing has been demonstrated to inform optimal genome editing timeframes and expansion conditions that enhance CAR T potency and persistence. Robust correlations have been established between OMI and gene transfer efficiency by retroviral vectors or electroporation-based CRISPR and validated OMI measurements with standard metabolic assays such as metabolite plate-based assays and extracellular flux analysis. These findings support the applications of OMI to address the current technology needs in CAR T manufacturing (54).

### Materials and Methods

### Study Design

Sample size for each in vitro experiment includes at least 3 donors with at least 50 cells/experimental group/donor to capture intra- and inter-donor heterogeneity based on a previous power analysis for the sensitivity of OMI to T cell activation (35). For the mouse studies, two donors were used across two independent experiments and the number of mice per group was determined by prior study on the same virus-free CRISPR-edited anti-GD2 CAR T model (9), with at least 4 mice in each group receiving either media-transitioned (Tex→Imm) CAR T cells or non-media-transitioned (Imm or Tex) CAR T cells. Rules for stopping data collection was based on the 10-day CAR T cell manufacturing process set by prior protocols (9). Mouse tumors were followed until tumor volume reached 4000 mm³. No data besides the mouse studies, in which one mouse was excluded due to out-of-range starting tumor size as determined by IVIS reading. Otherwise outliers have not been removed. Primary and secondary endpoints were prospectively selected, and appropriate statistical corrections were applied to multiple endpoints. The number of repeats for each experiment is given in the figure caption to show that results were substantiated across multiple donors. Replication is performed at several levels: cell, image, and donor, with 4-7 technical replicates (images) per experiment. Each main finding of the paper is supported by 650-4,800 T cells from two to three donors (findings include: OMI endpoints for classification and correlation analysis to determine impacts of media composition on T cell activation, and to predict cell cycle entry, optimal gene transfer conditions, and CAR T metabolic fitness). Supportive experiments (ATP production, cellular reducing potential, extracellular flux analysis, lactate measurement) include at least 3 technical replicates from 1-2 donors.

The objectives of the disclosed research were to demonstrate that OMI can determine (1) cell cycle entry timeline, (2) optimal CAR gene transfer conditions, and (3) media compositions that enhance metabolic fitness of CAR T cells. These were pre-specified objectives. Research subjects were healthy volunteers and NSG mice bearing GD2+ Luciferase CHLA-20 tumors. This was a controlled laboratory experiment where treatments and measurement techniques are described below. Mice were randomly assigned among treatment groups as described below.

### T Cell Activation with Different Activating Antibody and Media

CD3 T cells were isolated from peripheral blood of 3 healthy donors as previously described and plated at 1 million cells/mL. T cells were divided into 4 groups and activated with 4 unique combinations of media (ImmunoCult XF media or TexMACS media) and stimulating antibodies (25 μL/mL StemCell αCD2/αCD3/αCD28 or 10 μL/mL TransAct αCD3/αCD28). To focus on the specific impacts of media and activating antibodies on T cell metabolism and metabolic shift kinetics, no cytokine was supplemented to activated T cells. Activated T cells were imaged using OMI at 24, 48, and 72 hours.

### Virus-Free Anti-GD2 CAR T Cell Generation and Activation Time Course Setup

Peripheral blood was drawn from healthy donors under a protocol approved by the Institutional Review Board at the University of Wisconsin-Madison (2018-0103) and informed consent was obtained from all donors. CD3 T cells were isolated by negative selection following manufacturer's protocol (RosetteSep Human T cell enrichment cocktail, STEMCELL Technologies). Following isolation, T cells were plated at 1 million cells/mL in either ImmunoCult XF T cell Expansion Medium (STEMCELL Technologies) supplemented with 200U/mL IL-2 (Peprotech) or TexMACS Cell Culture Medium (Miltenyi Biotec) supplemented with 10 ng IL-7 (Biotechne), and stimulated with 25 μL/mL ImmunoCult Human αCD2/αCD3/αCD28 T cell Activator (STEMCELL Technologies) or 10 μL/mL T-cell TransAct αCD3/αCD28 (Miltenyi Biotec), respectively. These two combinations of activating antibodies and culture media yielded two activation methods, Imm and Tex respectively. T cells were activated for different durations, ranging from 12 hours up to 72 hours. The metabolic profile of activated T cells was characterized with OMI while cell cycle stage and proliferative capacity were analyzed via flow cytometry of Hoechst and Ki67 staining, respectively. Cell cycle stages were gated based on median fluorescence intensity (MFI) of Hoechst stain that reflected cellular DNA content. These activated T cells were then electroporated with CRISPR/Cas9 machinery to express anti-GD2 CAR transgene as previously described (9). Following electroporation, Imm- and Tex-activated anti-GD2 CAR T cells were expanded for 7 days in ImmunoCult XF T cell expansion media supplemented with 500 U/mL IL-2 or TexMACS media supplemented with 10 ng IL-7, respectively. After expansion, CAR T cells were harvested; percent CAR positivity was quantified via flow cytometry of 1A7 anti-14G2A antibody (National Cancer Institute, Biological Resources Branch) conjugated to APC using a Lightning Link APC Antibody Labeling kit (Novus Biologicals) to evaluate genome editing efficiency as previously described (FIG. S1) (9, 41).

### Virus-Free Anti-GD2 CAR T Cell Expansion and Flow Cytometry Characterization

T cells from 3 donors were activated with either Imm or Tex method prior to electroporation with CRISPR/Cas9 machinery to express anti-GD2 CAR as previously described (9, 41). Following electroporation, CAR T cells were expanded in either ImmunoCult XF or TexMACS media for 7 days in a 37 C, 5% CO₂ humidified incubator. During expansion, T cell media were supplemented with 500 U/mL IL-2 (Peprotech), 10 ng/ml IL-7 (BioTechne), 10 ng/ml IL-7 (BioTechne)+10 ng/ml IL-15 (BioTechne) (IL-7/IL-15 low), 10 ng/ml IL-7 (BioTechne)+1 ng/ml IL-15 (BioTechne) (IL-7/IL-15 low), or 10 ng/ml IL-7 (BioTechne)+100U/mL IL-2 (Peprotech) (IL-7/IL-2). Cells were counted every 2 days and adjusted to 1 million cells/mL. CAR T cell phenotypes and metabolism were analyzed with flow cytometry and OMI after 7 days of expansion. Flow cytometry was performed on an Aurora spectral cytometer (Cytek) as previously described (9, 41). Briefly, T cells were stained and analyzed for expression of 16 markers (anti-GD2 CAR, TCRαβ, CD4, CD8, CD45RA, CD45RO, CD62L, CCR7, Human CD45, PD-1, LAG3, TIM3, CD39, TIGIT, CD27, CD5) with specific clones and concentrations as previously described (41). An expression histogram of each surface marker was generated based on single-cell MFIs normalized by donor-matched fluorescence-minus-one (FMO) control. For in vivo treatment, 3 groups of CAR T cells with different expansion conditions were generated. Imm CAR T cells were activated in ImmunoCult XF media+200U/mL IL-2 and expanded in ImmunoCult XF media+500U/mL IL-2 following electroporation. Tex CAR T cells were activated and expanded in TexMACS media+10 ng/mL IL-7. Meanwhile, Tex→Imm CAR T cells were activated in TexMACS media+10 ng/ml IL-7 and expanded in ImmunoCult XF media+500U/mL IL-2 after electroporation.

### OMI of T Cells

T cells were plated on a 35 mm Poly-D-Lysine glass bottom imaging dish (MatTek) at the density of 200,000 cells/75 μL and allowed to settle for at least 15 minutes prior to imaging. Throughout the process of OMI, T cells were kept in a stage top incubator (37° C., 5% CO₂) to maintain their physiological conditions. OMI was performed on a custom-built multiphoton microscope (Ultima, Bruker) consisting of an inverted microscope body (Ti-E, Nikon) coupled to an ultrafast tunable laser source (Insight DS+, Spectra Physics). Images were acquired using time-correlated single-photon counting electronics (SPC 150, Becker & Hickl GmbH) using Prairie View Software (Bruker). NAD(P)H and FAD were excited at 750 nm (2.5 mW) and 890 nm (4.5 mW), respectively, using a 40× water immersion 1.15 NA objective (Nikon) with 2.5× optical zoom, 4.8 μs pixel dwell time, 60s integration time, and image size of 256×256 pixels. NAD(P)H and FAD emission were separated from excitation light using a 720 long pass filter and collected using GaAsP photomultiplier tubes (H7422, Hamamatsu) through a 440/80 nm and 550/100 nm bandpass filters, respectively. PerCP conjugated CAR antibodies were excited at 980 nm, while Alexa 647 CCR7 antibody was excited at 1200 nm, respectively. Fluorescence emission of PerCP and Alexa 647 were collected with 690/50 nm filter. Fluorescence intensity and lifetime images of NAD(P)H and FAD, together with immunofluorescence images of surface markers, were collected for each field of view (FOV), with 3-5 representative FOVs (~150-250 cells, ~120 μm×120 μm) imaged per condition. The instrument response function was measured using second-harmonic generation signal from urea crystals excited at 890 nm, with full width at half-maximum of 260 ps.

### OMI Analysis

Fluorescence lifetime components were computed for each image pixel using SPCImage (v8.0, Becker and Hickl GmbH) by first thresholding the background, then the pixel-wise decay curves were fit to a biexponential model convolved with the instrument response function, using an iterative parameter optimization to obtain the lowest sum of the squared differences between model and data (Weighted Least Squares algorithm). The two-component exponential decay model is

$$I ( t ) = {α}_{1} {e}^{- t / {τ}_{1}} + {α}_{2} {e}^{- t / {τ}_{2}} + C ,$$

where I(t) is the fluorescence intensity at time t after the laser excitation pulse, τ₁ and τ₂ are the fluorescence lifetimes of the short and long lifetime components, respectively, α₁ and α₂ are the fractional contributions of the short and long lifetime components, respectively, and C accounts for background light. The mean fluorescence lifetime, τₘ=α₁τ₁+α₂τ₂, is the weighted average of the free- and protein-bound-fraction (55, 56). To enhance the fluorescence counts in each decay, a bin of 1 (comprising 9 neighbor pixels) and of a bin of 2 (comprising 25 neighbor pixels) were applied on NAD(P)H and FAD lifetime images, respectively. The pixel-wise optical redox ratio was calculated as

$$\frac{NAD ( P ) {H}_{i ntensity}}{NAD ( P ) {H}_{i ntensity} + {FAD}_{i ntensity}} .$$

A customized CellProfiler pipeline was used for manual segmentation of every individual cell nucleus within a FOV, from which the individual cell border was propagated to create a whole-cell mask. A single-cell cytoplasm mask was generated by subtracting the nuclei mask from the whole cell mask. The cytoplasm mask was applied to the corresponding OMI image to compute mean values of OMI parameters for each cell cytoplasm. 13 OMI parameters were collected and quantified (Table 2). Cytoplasm size was also computed for individual cells based on cytoplasm masks.

### Extracellular Flux Analysis

Extracellular flux assay was performed using Seahorse XF Cell Mito Stress Test Kit (Agilent). 24 hours prior to the assay, a Seahorse cell culture 96 well-plate (Agilent) was coated with 50 μL/well of 50 μg/mL Gibco Poly-D-lysine (ThermoFisher Scientific) for 1 hour, then washed with distilled water before storing overnight at 4 C. The cell culture plate was equilibrated to room temperature before cell plating. 400,000 T cells/well were plated onto Poly-D-lysine coated cell culture plate in RPMI XF media (Agilent) supplemented with 10 mM glucose (Agilent) and 2 mM glutamine (Agilent) following the manufacturer's protocol for seeding suspension cells in Seahorse XFp cell culture miniplate. Briefly, the T cell culture plate was centrifuged at 200 g for 1 minute (no brake) and checked under the microscope to ensure even adhesion of T cells. T cells were then kept in a non-CO₂ incubator for at least 1 hour before running the assay. 1.5 μM FCCP, 2.5 μM Oligomycin, and 0.5 μM Antimycin A/Rotenone were loaded into port A, B, and C respectively as metabolic inhibitors. Oxygen consumption rate (OCR) and extracellular acidification rate (ECAR) were measured with an XF96 Extracellular Flux Analyzer (Seahorse Bioscience).

### Lactate Measurement

At harvest, 1 million CAR T cells for each condition were plated in 1 mL fresh media (similar to their expansion condition) in a tissue culture treated 24-well plate (VWR). After 24 hours, spent media was collected for each condition and lactate secretion was assayed in duplicate or triplicate using either Lactate Colorimetric/Fluorometric kit (abcam) or Lactate-Glo (Promega) following manufacturers' protocols. Lactate in base TexMACS and ImmunoCult media were measured as the baseline controls. For abcam's Lactate Colorimetric/Fluorometric kit (abcam), lactate secretion for each condition was measured based on the fluorescence intensity at 535/587 nm excitation/emission using Tecan M1000 plate reader. Fitting and extrapolating of standard curve and sample measurements were performed in GraphPad Prism v10 (linear curve fitting).

### ATP Production, Extracellular Metabolite Assays, and Cellular Reducing Capacity Assay

Pan T cells were isolated (Miltenyi) from PBMCs (Leukopak, StemCell Technologies) and plated in TexMACS or ImmunoCult at 7×10⁵ cells/ml in a 6-well plate. Cells were then subsequently activated with TransAct (Miltenyi), including a non-activated control. After activation, samples were taken and incubated with RealTime-Glo™ MT (Promega) per manufacturer's instructions in a CO₂ and temperature-controlled plate reader (Tecan Spark Cyto). Luminescence was recorded every hour for 74 hours. Additionally, daily samples were obtained, and ATP levels were determined using CellTiter-Glo® from Promega, following the manufacturer's instructions.

### Mouse CHLA-20 Xenograft and CAR T Cell Treatment

All animal experiments were approved by the University of Wisconsin-Madison Animal Care and Use Committee (ACUC protocol M005915). Establishment of CHLA-20 xenograft and subsequent CAR T cell treatments were performed as previously described (9, 41). Briefly, male and female NOD-SCID-γc^{-/-} (NSG™) mice (9-25 weeks old; Jackson Laboratory) received 10 million AkaLUC-GFP CHLA-20 GD2⁺ human neuroblastoma cells via subcutaneous flank injection to establish tumors. Tumor flux was measured with IVIS imaging one day prior to CAR T cell treatment, and mice were divided into three treatment groups to control for equally distributed tumor flux in each group. 4 million anti-GD2 CAR⁺ cells were injected into the tail vein of tumor-bearing each mouse. Tumor flux was monitored with IVIS imaging every 3-4 days. During CAR T treatment duration, mice also received 100,000 IU of human IL-2 (National Cancer Institute, Biological Resources Branch) subcutaneously on day 0 and post imaging. Total flux was calculated using Living Image Software (PerkinElmer) as radiance (photons/second) in each pixel integrated over tumor area (cm²)×4π. To normalize for background signal, the minimum flux value was subtracted from each image.

### Statistical Analysis

All statistical analysis was performed in Prism GraphPad v10, with appropriate statistical tests chosen based on data features. p<0.05 was chosen as the threshold for statistical significance. Multiple comparisons were adjusted with post-hoc tests. Glass's delta for effect size was calculated as

$$\frac{\vert {μ}_{treatment} - {μ}_{control} \vert}{{σ}_{control}}$$

where μₜᵣₑₐₜₘₑₙₜ and μ_{control} representing the means of treated and control groups, respectively; and σ_{control} being the standard deviation of the control group. MFI of surface markers) were normalized to donor-match control groups to account for any variations in laser power. Details on the number of sampled units and specific statistical tests for each experiment were reported in corresponding figure legends.

### REFERENCES—THE FOLLOWING REFERENCES AS WELL AS ANY REFERENCE CITED HEREIN ARE INCORPORATED BY REFERENCE HEREIN IN THEIR ENTIRETY

1. Melenhorst, J. J. et al. Decade-long leukaemia remissions with persistence of CD4+ CAR T cells. Nature 602, 503-509 (2022).
2. Guzman, G., Reed, M. R., Bielamowicz, K., Koss, B. & Rodriguez, A. CAR-T Therapies in Solid Tumors: Opportunities and Challenges. Curr. Oncol. Rep. 25, 479-489 (2023).
3. Stefanski, H. E. et al. Higher doses of tisagenlecleucel are associated with improved outcomes: a report from the pediatric real-world CAR consortium. Blood Adv. 7, 541-548 (2023).
4. Xu, H. et al. Influence of various medium environment to in vitro human T cell culture. In Vitro Cell. Dev. Biol. Anim. 54, 559-566 (2018).
5. Li, Y. & Kurlander, R. J. Comparison of anti-CD3 and anti-CD28-coated beads with soluble anti-CD3 for expanding human T cells: Differing impact on CD8 T cell phenotype and responsiveness to restimulation. J. Transl. Med. 8, 104 (2010).
6. Fesnak, A. D. The Challenge of Variability in Chimeric Antigen Receptor T cell Manufacturing. Regen. Eng. Transl. Med. 6, 322-329 (2020).
7. Abou-el-Enein, M. et al. Scalable Manufacturing of CAR T Cells for Cancer Immunotherapy. Blood Cancer Discov. 2, 408-422 (2021).
8. Research, C. for B. E. and. FDA Investigating Serious Risk of T-cell Malignancy Following BCMA-Directed or CD19-Directed Autologous Chimeric Antigen Receptor (CAR) T cell Immunotherapies. FDA (2023).
9. Mueller, K. P. et al. Production and characterization of virus-free, CRISPR-CAR T cells capable of inducing solid tumor regression. J. Immunother. Cancer 10, e004446 (2022).
10. Hu, Y. et al. Safety and efficacy of CRISPR-based non-viral PD1 locus specifically integrated anti-CD19 CAR-T cells in patients with relapsed or refractory Non-Hodgkin's lymphoma: a first-in-human phase I study. eClinicalMedicine 60, (2023).
11. Balke-Want, H. et al. Homology-independent targeted insertion (HITI) enables guided CAR knock-in and efficient clinical scale CAR-T cell manufacturing. Mol. Cancer 22, 100 (2023).
12. Caruso, H. G. et al. Shortened ex vivo manufacturing time of EGFRvIII-specific chimeric antigen receptor (CAR) T cells reduces immune exhaustion and enhances antiglioma therapeutic function. J. Neurooncol. 145, 429-439 (2019).
13. Ghassemi, S. et al. Reducing Ex Vivo Culture Improves the Antileukemic Activity of Chimeric Antigen Receptor (CAR) T Cells. Cancer Immunol. Res. 6, 1100-1109 (2018).
14. Shin, J. J. et al. Controlled Cycling and Quiescence Enables Efficient HDR in Engraftment-Enriched Adult Hematopoietic Stem and Progenitor Cells. Cell Rep. 32, 108093 (2020).
15. Smirnikhina, S. A., Zaynitdinova, M. I., Sergeeva, V. A. & Lavrov, A. V. Improving Homology-Directed Repair in Genome Editing Experiments by Influencing the Cell Cycle. Int. J. Mol. Sci. 23, 5992 (2022).
16. Watanabe, N., Mo, F. & McKenna, M. K. Impact of Manufacturing Procedures on CAR T Cell Functionality. Front. Immunol. 13, 876339 (2022).
17. Wang, F., Cheng, F. & Zheng, F. Stem cell like memory T cells: A new paradigm in cancer immunotherapy. Clin. Immunol. 241, 109078 (2022).
18. López-Cantillo, G., Urueña, C., Camacho, B. A. & Ramírez-Segura, C. CAR-T Cell Performance: How to Improve Their Persistence? Front. Immunol. 13, 878209 (2022).
19. Zhou, J. et al. Chimeric antigen receptor T (CAR-T) cells expanded with IL-7/IL-15 mediate superior antitumor effects. Protein Cell 10, 764-769 (2019).
20. Nanjireddy, P. M., Olejniczak, S. H. & Buxbaum, N. P. Targeting of chimeric antigen receptor T cell metabolism to improve therapeutic outcomes. Front. Immunol. 14, 1121565 (2023).
21. van Bruggen, J. A. C. et al. Chronic lymphocytic leukemia cells impair mitochondrial fitness in CD8+ T cells and impede CAR T-cell efficacy. Blood 134, 44-58 (2019).
22. Sukumar, M. et al. Inhibiting glycolytic metabolism enhances CD8+ T cell memory and antitumor function. J. Clin. Invest. 123, 4479-4488 (2013).
23. Klein Geltink, R. I. et al. Metabolic conditioning of CD8+ effector T cells for adoptive cell therapy. Nat. Metab. 2, 703-716 (2020).
24. Walsh, A. J. et al. Classification of T-cell activation via autofluorescence lifetime imaging. Nat. Biomed. Eng. 5, 77-88 (2021).
25. Walsh, A. J. et al. Optical metabolic imaging identifies glycolytic levels, subtypes, and early-treatment response in breast cancer. Cancer Res. 73, 6164-6174 (2013).
26. Chance, B., Legallais, V. & Schoener, B. Metabolically Linked Changes in Fluorescence Emission Spectra of Cortex of Rat Brain, Kidney and Adrenal Gland. Nature 195, 1073-1075 (1962).
27. Skala, M. C. et al. In vivo multiphoton microscopy of NADH and FAD redox states, fluorescence lifetimes, and cellular morphology in precancerous epithelia. Proc. Natl. Acad. Sci. 104, 19494-19499 (2007).
28. Georgakoudi, I. & Quinn, K. P. Optical imaging using endogenous contrast to assess metabolic state. Annu. Rev. Biomed. Eng. 14, 351-367 (2012).
29. Quinn, K. P. et al. Quantitative metabolic imaging using endogenous fluorescence to detect stem cell differentiation. Sci. Rep. 3, 3432 (2013).
30. Shah, V. S. et al. Autofluorescence imaging permits label-free cell type assignment and reveals the dynamic formation of airway secretory cell associated antigen passages (SAPs). eLife 12, e84375 (2023).
31. Neuroblastoma Survival Rates American Cancer Society. https://www.cancer.org/cancer/types/neuroblastoma/detection-diagnosis-staging/survival-rates.html.
32. Richards, R. M., Sotillo, E. & Majzner, R. G. CAR T Cell Therapy for Neuroblastoma. Front. Immunol. 9, 2380 (2018).
33. Del Bufalo, F. et al. GD2-CART01 for Relapsed or Refractory High-Risk Neuroblastoma. N. Engl. J. Med. 388, 1284-1295 (2023).
34. MacPherson, S. et al. Clinically relevant T cell expansion media activate distinct metabolic programs uncoupled from cellular function. Mol. Ther.—Methods Clin. Dev. 24, 380-393 (2022).
35. Walsh, A. J. et al. Classification of T-cell activation via autofluorescence lifetime imaging. Nat. Biomed. Eng. 5, 77-88 (2021).
36. Pearce, E. L. Metabolism in T cell activation and differentiation. Curr. Opin. Immunol. 22, 314-320 (2010).
37. Wei, J., Raynor, J., Nguyen, T.-L. M. & Chi, H. Nutrient and Metabolic Sensing in T Cell Responses. Front. Immunol. 8, (2017).
38. Yang, Y. & Sauve, A. A. NAD+ metabolism: Bioenergetics, signaling and manipulation for therapy. Biochim. Biophys. Acta 1864, 1787-1800 (2016).
39. Hu, Y. et al. CRISPR/Cas9-Engineered Universal CD19/CD22 Dual-Targeted CAR-T Cell Therapy for Relapsed/Refractory B-cell Acute Lymphoblastic Leukemia. Clin. Cancer Res. 27, 2764-2772 (2021).
40. Khan, A. & Sarkar, E. CRISPR/Cas9 encouraged CAR-T cell immunotherapy reporting efficient and safe clinical results towards cancer. Cancer Treat. Res. Commun. 33, 100641 (2022).
41. Cappabianca, D. et al. Metabolic priming of GD2 TRAC-CAR T cells during manufacturing promotes memory phenotypes while enhancing persistence. 2024.01.31.575774 Preprint at https://doi.org/10.1101/2024.01.31.575774 (2024).
42. Xu, X., Gnanaprakasam, J. N. R., Sherman, J. & Wang, R. A Metabolism Toolbox for CAR T Therapy. Front. Oncol. 9, 322 (2019).
43. Nabe, S. et al. Reinforce the antitumor activity of CD8+ T cells via glutamine restriction. Cancer Sci. 109, 3737-3750 (2018).
44. Xu, L. et al. PD-1 and TIGIT Are Highly Co-Expressed on CD8+ T Cells in AML Patient Bone Marrow. Front. Oncol. 11, 686156 (2021).
45. Banta, K. L. et al. Mechanistic convergence of the TIGIT and PD-1 inhibitory pathways necessitates co-blockade to optimize anti-tumor CD8+ T cell responses. Immunity 55, 512-526.e9 (2022).
46. Zhao, M.-H., Kim, N.-H. & Cui, X.-S. GlutaMAX prolongs the shelf life of the culture medium for porcine parthenotes. Theriogenology 85, 368-375 (2016).
47. Long, A. H., Orentas, R. J. & Mackall, C. L. Synthetic Chimeric Antigen Receptors (CARs) Rapidly Induce Exhaustion and Augmented Glycolytic Metabolism In Human T Cells and Implicate Persistent CD28 Signaling As a Driver Of Exhaustion In Human T Cells. Blood 122, 192 (2013).
48. Scharping, N. E. et al. The Tumor Microenvironment Represses T Cell Mitochondrial Biogenesis to Drive Intratumoral T Cell Metabolic Insufficiency and Dysfunction. Immunity 45, 374-388 (2016).
49. DePeaux, K. & Delgoffe, G. M. Metabolic barriers to cancer immunotherapy. Nat. Rev. Immunol. 21, 785-797 (2021).
50. Mosier, J. A., Schwager, S. C., Boyajian, D. A. & Reinhart-King, C. A. Cancer cell metabolic plasticity in migration and metastasis. Clin. Exp. Metastasis 38, 343-359 (2021).
51. Lee, D.-H., Li, X., Ma, N., Digman, M. A. & Lee, A. P. Rapid and label-free identification of single leukemia cells from blood in a high-density microfluidic trapping array by fluorescence lifetime imaging microscopy. Lab. Chip 18, 1349-1358 (2018).
52. Samimi, K. et al. Time-domain single photon-excited autofluorescence lifetime for label-free detection of T cell activation. Opt. Lett. 46, 2168-2171 (2021).
53. Samimi, K. et al. Light-sheet autofluorescence lifetime imaging with a single-photon avalanche diode array. J. Biomed. Opt. 28, 066502 (2023).
54. Gillette, A. A., Pham, D. L. & Skala, M. C. Touch-free optical technologies to streamline the production of T cell therapies. Curr. Opin. Biomed. Eng. 25, 100434 (2023).
55. Lakowicz, J. R., Szmacinski, H., Nowaczyk, K. & Johnson, M. L. Fluorescence lifetime imaging of free and protein-bound NADH. Proc. Natl. Acad. Sci. U.S.A 89, (1992).
56. Datta, R., Heaster, T. M., Sharick, J. T., Gillette, A. A. & Skala, M. C. Fluorescence lifetime imaging microscopy: fundamentals and advances in instrumentation, analysis, and applications. J. Biomed. Opt. 25, (2020).

1. A method for gene editing of a T cell, comprising:
stimulating a T cell obtained from a subject;
obtaining a plurality of measurements of a metabolic indicator within the T cell at a respective plurality of timepoints after stimulating the T cell;
analyzing the plurality of measurements of the metabolic indicator to identify a particular time for performing gene editing of the T cell; and
providing an indication to perform gene editing of the T cell at the particular time based on analyzing the measurements.

2. The method of claim 1, wherein stimulating a T cell obtained from a subject further comprises:
stimulating a T cell obtained from a subject by contacting the T cell with an antibody reagent.

3. The method of claim 2, wherein the antibody reagent is at least one of ImmunoCult (αCD2/αCD3/αCD28) or TransAct (αCD3/αCD28) antibody reagents.

4. The method of claim 2, wherein obtaining a plurality of measurements further comprises:
obtaining a plurality of autofluorescence measurements from the T cell.

5. The method of claim 4, wherein obtaining a plurality of autofluorescence measurements further comprises:
stimulating the T cell using excitation light, and
obtaining at least one of photon counts/intensity or fluorescence lifetimes from the T cell.

6. The method of claim 5, wherein stimulating the T cell using excitation light further comprises:
stimulating the T cell using excitation light configured to be absorbed by at least one of NAD(P)H or FAD, and
wherein obtaining a plurality of autofluorescence measurements further comprises:
obtaining the plurality of autofluorescence measurements from at least one of NAD(P)H or FAD stimulated by excitation light in the T cell.

7. The method of claim 6, wherein obtaining a plurality of measurements of a metabolic indicator within the T cell at a respective plurality of timepoints after stimulating the T cell further comprises:
obtaining a plurality of measurements of autofluorescence decay from the T cell based on the plurality of measurements obtained at the respective plurality of timepoints,
wherein the autofluorescence decay comprises signal from at least one of NAD(P)H or FAD within the T cell, and
wherein analyzing the plurality of measurements of the metabolic indicator to identify a particular time for performing gene editing of the T cell further comprises:
identifying the metabolic indicator based on analyzing the autofluorescence decay from the T cell.

8. The method of claim 7, wherein identifying the metabolic indicator based on analyzing the autofluorescence decay from the T cell further comprises:
identifying the metabolic indicator based on using at least one of a curve-fitting or fit-free analysis of the autofluorescence decay,
wherein the autofluorescence decay is modeled as a multi-component exponential decay.

9. The method of claim 8, wherein the metabolic indicator comprises at least one of NAD(P)H mean fluorescence lifetime (τₘ) or NAD(P)H shortest fluorescence amplitude component (α₁), and
wherein analyzing the plurality of measurements of the metabolic indicator to identify a particular time for performing gene editing of the T cell further comprises:
obtaining a plurality of values of NAD(P)H τₘ and a plurality of values of NAD(P)H α₁ at each of the respective plurality of timepoints,
analyzing the plurality of values of NAD(P)H τₘ and the plurality of values of NAD(P)H α₁, and identifying the particular time for performing gene editing of the T cell based on analyzing the plurality of values of NAD(P)H τₘ and the plurality of values of NAD(P)H α₁.

10. The method of claim 9, wherein analyzing the plurality of values of NAD(P)H τₘ and the plurality of values of NAD(P)H α₁ further comprises:
identifying at least one of a minimum value of the plurality of values of NAD(P)H τₘ or a maximum value of the plurality of values of NAD(P)H α₁, and
identifying the particular time for performing gene editing of the T cell based on identifying at least one of a minimum value of the plurality of values of NAD(P)H τₘ or a maximum value of the plurality of values of NAD(P)H α₁.

11. The method of claim 10, wherein the particular time for performing gene editing of the T cell corresponds to a time when the T cell is at the G2/M cell cycle phase.

12. The method of claim 1, wherein performing gene editing of the T cell at the particular time based on analyzing the measurements further comprises:
adding a chimeric antigen receptor to the T cell using at least one of CRISPR or viral editing gene editing procedures.

13. The method of claim 4, wherein obtaining a plurality of autofluorescence measurements from the T cell further comprises:
obtaining a plurality of measurements of a cytoplasm size or a cell size for the T cell at each of the plurality of timepoints,
identifying a maximum value of the cytoplasm size or the cell size on the plurality of measurements of the cytoplasm size or the cell size, and
identifying the particular time for performing gene editing based on identifying the maximum value of the cytoplasm size or the cell size.

14. The method of claim 1, further comprising infusing the T cell into the subject.

15. The method of claim 1, wherein obtaining a plurality of measurements of a metabolic indicator within the T cell further comprises:
obtaining the plurality of measurements of the metabolic indicator within the T cell while the T cell is contained within an observation zone.

16. (canceled)

17. (canceled)

18. A system for gene editing of a T cell, comprising:
an observation zone comprising a T cell obtained from a subject,
the T cell being stimulated at an initial time;
a spectrometer configured to obtain a plurality of measurements of a metabolic indicator within the T cell at a respective plurality of timepoints after stimulating the T cell at the initial time;
a processor in electronic communication with the spectrometer; and
a non-transitory computer-readable medium accessible to the processor and having stored thereon instructions that, when executed by the processor, cause the processor to:
analyze the plurality of measurements of the metabolic indicator to identify a particular time for performing gene editing of the T cell, and
provide an indication of the particular time to perform gene editing of the T cell based on analyzing the measurements.

19. The system of claim 18, wherein the T cell obtained from the subject is stimulated by contacting the T cell with at least one of ImmunoCult (αCD2/αCD3/αCD28) or TransAct (αCD3/αCD28) antibody reagents.

20. The system of claim 19, wherein the spectrometer, when obtaining a plurality of measurements, is further configured to:
obtain a plurality of autofluorescence measurements from the T cell.

21-27. (canceled)

28. The system of claim 18, wherein the processor, when providing an indication of the particular time to perform gene editing of the T cell, is further caused by the instructions to:
provide an instruction to add a chimeric antigen receptor to the T cell using at least one of CRISPR or viral editing gene editing procedures.

29. (canceled)

30. The system of claim 18, wherein the T cell is infused into the subject.

31-59. (canceled)
